(19) United States
(12) Patent Application Publication
(10) Pub. No.: US 2025/0281413 A1
(43) Pub. Date: Sep. 11, 2025

(54) PHARMACEUTICAL COMPOSITION

(71) Applicant: Novartis AG, Basel (CH)

(72) Inventors: Michael JUHNKE, Basel (CH); Karin RAPP, Lörrach (DE); Kim-Hien SIN, Saint-Louis (FR)

(21) Appl. No.: 18/261,523

(22) PCT Filed: Jan. 24, 2022
(86) PCT No.: PCT/IB2022/050578

§ 371 Date: Jul. 14, 2023

Related U.S. Application Data
(60) Provisional application No. 63/141,558, filed on Jan. 26, 2021.
(60) Provisional application No. 63/240,438, filed on Sep. 3, 2021.
(60) Provisional application No. 63/290,251, filed on Dec. 16, 2021.

(51) Int. Cl. A61K 9/28; A61K 9/00; A61K 9/16; A61K 9/20; A61K 9/48; A61K 31/505

(52) U.S. Cl. CPC A61K 9/2833; A61K 9/0053; A61K 9/1617; A61K 9/1623; A61K 9/1635; A61K 9/2013; A61K 9/2027; A61K 9/4825; A61K 31/505

(57) ABSTRACT

The present invention relates to the field of pharmacy, particularly to a pharmaceutical composition for oral administration comprising a pharmaceutical composition for oral administration comprising: (a) an inert substrate, and (b) a mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, and at least one binder. The present invention also relates to a process for preparing said pharmaceutical composition for oral administration; and to the use of said pharmaceutical composition in the manufacture of a medicament.

### FIELD OF THE INVENTION

The present invention relates to the field of pharmacy, particularly to a pharmaceutical composition for oral administration comprising: (a) an inert substrate, and (b) a mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, and at least one binder. The present invention also relates to a process for preparing said pharmaceutical composition for oral administration; and to the use of said pharmaceutical composition in the manufacture of a medicament.

### BACKGROUND OF THE INVENTION

Bruton's tyrosine kinase (BTK) is a cytoplasmic tyrosine kinase and member of the TEC kinase family (Smith et al, BioEssays, 2001, 23, 436-446). BTK is expressed in selected cells of the adaptive and innate immune system including B cells, macrophages, mast cells, basophils and thrombocytes.

The essential role of BTK in autoimmune disease is underlined by the observations that BTK-deficient mice are protected in standard preclinical models of rheumatoid arthritis (Jansson and Holmdahl, Clin. Exp. Immunol. 1993, 94, 459-465), systemic lupus erythematosus, as well as allergic disease and anaphylaxis. In addition, many cancers and lymphomas expressing BTK appear to be dependent on BTK function (Davis et al. Nature, 2010, 463, 88-92). The role of BTK in diseases including autoimmunity, inflammation and cancer has been recently reviewed (Tan et al, Pharmacol. Ther., 2013, 294-309; Whang et al, Drug Discov. Today, 2014, 1200-4).

The specific BTK inhibitor N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, is referred to as Compound (A) of formula:

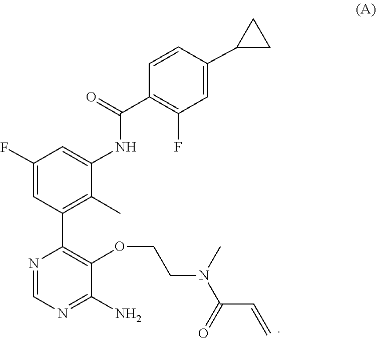 [SMILES: C=CC(=O)N(C)CCOc1c(N)ncnc1-c1cc(F)cc(NC(=O)c2ccc(C3CC3)cc2F)c1C]

Compound (A) is a selective potent irreversible covalent BTK inhibitor and is among a new generation of designed covalent enzyme inhibitors. Compound (A) was first disclosed in example 6 of WO2015/079417, filed Nov. 28, 2014 (attorney docket number PAT056021-WO-PCT) which is incorporated by reference in its entirety. Compound A is known as LOU064 which has the INN name of Remibrutinib. Said compound may be used for the treatment or prevention of a disease or disorder mediated by BTK or ameliorated by inhibition of BTK. Thus, there is a need to provide a commercially viable pharmaceutical composition comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof.

### BRIEF DESCRIPTION OF THE FIGURES

FIG. 1 shows the dissolution rate profile of the granule particles comprising Compound (A) at pH 2 (paddle 50 rpm).

FIG. 2 shows the dissolution rate profile of the granule particles comprising Compound (A) at pH 3 (paddle 50 rpm).

FIG. 3 depicts the pharmacokinetic (PK) profile of the granule particles comprising Compound (A) in dogs, at pH 2 (HCl 0.01 N).

FIG. 4 depicts the pharmacokinetic (PK) profile of the granule particles comprising Compound (A) in dogs, at pH 3 (HCl 0.01 N).

FIG. 5 depicts the pharmacokinetic (PK) profile of the granule particles comprising Compound (A) in dogs at pH 4.5 (acetate buffer), paddle 50 rpm.

FIG. 6 depicts the pharmacokinetic (PK) profile of the granule particles comprising Compound (A) in dogs, at pH 6.8 (phosphate buffer), paddle 50 rpm.

FIG. 7 shows the impact of the particle size of Compound (A) on the dissolution rate at pH 2 (paddle 50 rpm).

FIG. 8 shows the impact of the particle size of Compound (A) on the dissolution rate at pH 3 (paddle 50 rpm).

FIG. 9 depicts the pharmacokinetic (PK) profile in dogs using granule particles comprising micron-sized Compound (A) or nano-sized Compound (A).

FIG. 10 depicts the pharmacokinetic (PK) profile in dogs using granule particles comprising micron-sized Compound (A) or nano-sized Compound (A)—Semilogarithmic view

FIG. 11 depicts the scanning electron micrographs (SEM) of a wet milled suspension comprising Compound (A).

FIG. 12 depicts the dynamic viscosity of a wet-media milled suspension comprising Compound (A).

FIG. 13 depicts the scanning electron micrographs (SEM) of the wet-media milled suspensions comprising Compound (A), used for the F2, F5 and F6 formulations.

FIG. 14 depicts the scanning electron micrographs (SEM) of the wet-media milled suspension comprising Compound (A), used for the F7, F8 and F9 formulations.

FIG. 15 depicts the dynamic viscosity of different wet-media milled suspensions comprising 25% w/w Compound (A), used for optimization trials at 40° C. (FIG. 15a), 25° C. (FIG. 15b) and 10° C. (FIG. 15c).

FIG. 16 depicts Pareto charts showing the six most influencing factors on the blend particle size. (FIG. 16A and FIG. 16B)

FIG. 17 depicts Pareto charts showing the three most influencing factors on the blend bulk and density.

FIG. 18 depicts the flow properties according to the Pharmacopeia flowability scale (Carr's index below 25% and Hausner ratio 1.31) of various external phase composition.

FIG. 19 depicts Pareto charts showing the two most influencing factors for tensile strength of the tablet.

FIG. 20 depicts Pareto charts showing the main influencing factors for ejection force of the tablet.

FIG. 21 depicts Disintegration time of tablet cores in HCl, 0.01N pH2 for different formulations.

FIG. 22 depicts Pareto charts showing main influencing factors for dissolution rate.

FIG. 23 depicts Pareto charts showing main influencing factors on the particle size distribution of the granules. (FIG. 23A and FIG. 23B)

FIG. 24 depicts Pareto charts showing main influencing factors on granule bulk and tapped density.

FIG. 25 depicts the flow properties according to the Pharmacopeia flowability scale (Carr's index below 15% and Hausner ratio below 1.18) of different granule composition

FIG. 26 depicts Pareto charts showing main influencing factors on the granule flowability.

FIG. 27 depicts Pareto charts showing main influencing factors on Tensile strength at 30 kN compression force

FIG. 28 depicts Pareto charts showing main influencing factors on granule ejection force @30 kN

FIG. 29 depicts Pareto charts showing main influencing factors on Final blends PSD. (FIG. 29A and FIG. 29B)

FIG. 30 depicts the flow properties according to the Pharmacopeia flowability scale (Carr's index below 15% and Hausner ratio below 1.18) of final blend

FIG. 31 depicts Pareto charts showing main influencing factors on final blend flowability.

FIG. 32 depicts Granules and final blends sieving segregation profiles.

FIG. 33 depicts Pareto charts showing main influencing factors on the tablet tensile strength at 20 kN compression force FIG. 34 depicts Pareto charts showing main influencing factors on the tablet ejection force @20 kN.

FIG. 35 depicts Pareto charts showing main influencing factors on the Tablet core disintegration time in pH2

FIG. 36 depicts 2-way interaction graphs: disintegration time of 90N tablet cores

FIG. 37 depicts Pareto charts showing main influencing factors on Mean dissolution (90N and 120N) (FIG. 37A and FIG. 37B)

FIG. 38 depicts 2-way interaction graphs: dissolution rate for various drug load and copovidone load.

FIG. 39 depicts the evolution of the average particle size of Compound (A) against specific energy for several batches processed at process conditions wherein product temperature is of about 34 to about 40° C. and at an air to liquid ratio of about 2.0 to about 3.2; and with batch sizes (M) from about 62 to 175 kg, and process parameters rotor tip speed (v) from 10 to 14 m/s and suspension flow rate (V) from 5 to 20 L/min. The average particle size for Compound (A) was determined by Photon Correlation Spectroscopy (PCS) analysis.

FIG. 40 depicts the Loss on drying (LOD) trajectories of the granules during processing for batches processed at process conditions with different product temperature between 34 to 40° C. (T), spray rate (m), atomization air pressure (p) and air mass flow to liquid mass flow ratio, respectively air to liquid ratio (A/L) between about 2.0 to about 3.2; LOD was determined offline (offline LOD) from granule samples taken during processing using a halogen moisture analyzer and online (online LOD) from the fluidized granules during processing using a Near Infrared (NIR) spectroscopy probe installed in the fluid bed spray granulation equipment.

FIG. 41 depicts the particle size distributions of the granules produced from the process conditions with different product temperature between 34 and 40° C. (T), spray rate (m), atomization air pressure (p) and air mass flow to liquid mass flow ratio, respectively air to liquid ratio (A/L) between about 2.0 to about 3.2, corresponding to the experimental results shown in FIG. 40; The granule particle size distributions were determined by sieve analysis.

### SUMMARY OF THE INVENTION

The design of a pharmaceutical composition, a pharmaceutical dosage form, as well as a commercially viable process to prepare the pharmaceutical composition, for a BTK inhibitor such as N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, (herein referred as Compound (A)) is challenging. This BTK inhibitor is difficult to formulate due to its physicochemical properties, e.g. low solubility, low exposure, the compound had some gelling tendencies at certain pH conditions and was unstable when exposed to some temperatures and/or UV light. Ultimately, those issues were affecting the manufacturing process, but also the bioavailability and dispersibility of said BTK inhibitor of the present invention.

Accordingly, a suitable and robust solid pharmaceutical composition overcoming the above problems needs to be developed. The invention provides pharmaceutical composition with enhanced drug dissolution rate, increased absorption, increase of bioavailability, and a decrease of patient to patient variability. Furthermore, the invention provides a process for making the pharmaceutical composition, wherein such process provides an ease of scale up, a robust processing and economic advantages.

In view of the above-mentioned difficulties, and considerations, it was surprising to find a way to prepare a stable pharmaceutical composition that allows the preparation of a pharmaceutical composition comprising: (a) an inert substrate, and (b) a mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, and at least one binder.

Aspects, advantageous features and preferred embodiments of the present invention summarized in the following items, respectively alone or in combination, contribute to solving the object of the invention.

### EMBODIMENTS

1. A pharmaceutical composition for oral administration comprising a granule particle said granule particle comprising:
   (a) an inert substrate, and
   (b) a mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, and at least one binder.
2. The pharmaceutical composition according to embodiment 1, wherein N-(3-(6-amino-5-(2-(N-methylacrylamido)ethoxy)pyrimidin-4-yl)-5-fluoro-2-methylphenyl)-4-cyclopropyl-2-fluorobenzamide is in a free form.
3. The pharmaceutical composition according to embodiment 1 or 2, wherein the (b) mixture optionally further comprises a surfactant.
4. The pharmaceutical composition according to any one of embodiments 1-3, wherein the (b) mixture and optional surfactant, is layered onto the (a) inert substrate.
5. The pharmaceutical composition according to embodiment 4, wherein the (b) mixture and optional surfactant is layered onto the (a) inert substrate using a spray granulation method.
6. The pharmaceutical composition according to any one of embodiments 1-5, wherein the (a) inert substrate comprises a material which is selected from the group consisting of lactose, microcrystalline cellulose, mannitol, sucrose, starch, granulated hydrophilic fumed silica, or mixtures thereof, preferably the material, which is selected from the group consisting of lactose, mannitol, or mixtures thereof and most preferably the material is mannitol.
7. The pharmaceutical composition according to any one of embodiments 1-6, wherein the binder is independently selected from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, vitamin E Polyethylene Glycol succinate or a mixture thereof, preferably the binder is polyvinylpyrrolidone-vinyl acetate copolymer.
8. The pharmaceutical composition according to any one of embodiments 1-7, wherein the surfactant is selected from the group consisting of sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof, preferably the surfactant is sodium lauryl sulfate.
9. The pharmaceutical composition according to any one of embodiments 1-8, wherein the (b) mixture comprises N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, polyvinylpyrrolidone-vinyl acetate copolymer as a binder, and optionally sodium lauryl sulfate as a surfactant.
10. The pharmaceutical composition according to any one of embodiments 1-9, wherein the weight ratio between N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof, and the binder is about [3:1], about [2:1], about [1:1], about [1:2] or about [1:3] preferably about [1:1] and more preferably about [2:1].
11. The pharmaceutical composition according to any one of embodiments 1-9, wherein the weight ratio of N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof, the binder and the surfactant is [3:1:1], or about [3:1:0.5], or about [3:1:0.1], or about [2:1:1], or about [2:1:0.5], or about [2:1:0.1], or about [2:1:0.08], or about [2:1:0.05], or about [2:1:0.04], or about [2:1:0.03], or about [2:1:0.02], or about [1:1:0.5], or about [1:1:0.1], or about [1:1:0.07], or about [1:1:0.05], or about [1:1:0.04], or about [1:1:0.02], or about [1:3:0.1], or about [1:3:0.2], or about [1:1.5:0.25], preferably, the ratio is about [2:1:1], or about [2:1:0.5], or about [2:1:0.1], or about [2:1:0.08], or about [2:1:0.05], or about [2:1:0.04], or about [2:1:0.03], or about [2:1:0.02], or about [1:1:0.5], or about [1:1:0.1], or about [1:1:0.07], or about [1:1:0.05], or about [1:1:0.04], or about [1:1:0.02], and more preferably, the ratio is about [2:1:1], or about [2:1:0.08], or about [2:1:0.5], or about [2:1:0.1], or about [2:1:0.05], or about [2:1:0.04], or about [2:1:0.03], or about [2:1:0.02].
12. The pharmaceutical composition according to any one of embodiments 1-11, wherein the binder (e.g. polyvinylpyrrolidone-vinyl acetate copolymer) is present in the (b) mixture in an amount of 25% w/w to about 100% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, preferably about 50% w/w or about 100% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof.
13. The pharmaceutical composition according to any one of embodiments 1-12, wherein the (b) mixture further comprises a surfactant (e.g. Sodium lauryl sulfate) in an amount of 1% w/w to about 10% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, preferably about 4% w/w or about 5% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof.
14. The pharmaceutical composition according to any one of embodiments 1-13, wherein the particle size of said N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is less than 1000 nm.
15. The pharmaceutical composition according to embodiment 14, wherein the particle size of said N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is less than 500 nm.
16. The pharmaceutical composition according to embodiment 15, wherein the particle size of said N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is less than 350 nm, preferably less than 250 nm.
17. The pharmaceutical composition according to embodiment 14, wherein the particle size of said N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof as measured by PCS is between about 100 nm to about 350 nm; preferably between about 110 nm to about 180 nm.
18. The pharmaceutical composition according to any one of embodiments 1-17 further comprising an external phase wherein the external phase comprises one or more pharmaceutically acceptable excipient.
19. The pharmaceutical composition according to embodiment 18, wherein the one or more pharmaceutically acceptable excipient is selected from a filler, a disintegrating agent, a lubricating agent, and a gliding agent.
20. The pharmaceutical composition according to embodiment 18 or 19 wherein the external phase comprises one or more filler selected from calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof preferably mannitol or cellulose or mixture thereof.
21. The pharmaceutical composition according to any one of embodiments 18-20 wherein the external phase comprises one or more disintegrating agent selected from croscarmellose sodium, crospovidone, sodium starch glycolate, corn starch, or alginic acid, or mixture thereof.
22. The pharmaceutical composition according to any one of embodiments 18-21 wherein the external phase comprises one or more lubricating agent selected from magnesium stearate, sodium stearyl fumarate, stearic acid or talc or mixture thereof.
23. The pharmaceutical composition according to any one of embodiments 18-22 wherein the external phase comprises Mannitol and cellulose as fillers, sodium stearyl fumarate or magnesium stearate as lubricant, and croscarmellose sodium or sodium carbonate as disintegrating agent.
24. The pharmaceutical composition according to any one of embodiments 18-23 wherein the external phase is present in a 20-50% w/w/amount of to the total weight of the composition, preferably 40% w/w/amount of to the total weight of the composition.
25. The pharmaceutical composition according to any one of embodiments 1-24, wherein the pharmaceutical composition is further formulated into a final dosage form, optionally in the presence of at least one pharmaceutically acceptable excipient, and wherein said final dosage form is a capsule, a tablet, a sachet, or a stickpack.
26. The pharmaceutical composition according to embodiment 25, wherein the final dosage form is a capsule or preferably a tablet.
27. The pharmaceutical composition according to embodiment 25 or 26, wherein the capsule is selected from hard shell capsule, hard gelatin capsule, soft shell capsule, soft gelatin capsule, plant-based shell capsule, or a mixture thereof and wherein a tablet is preferably a film coated tablet.
28. A final dosage form which is a capsule formulation comprising a pharmaceutical composition of any one of embodiments 1-25.
29. A final dosage form with is a tablet formulation comprising a pharmaceutical composition of any one of embodiments 1-25.
30. A final dosage form according to embodiment 29, wherein N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is present in an amount of about 0.4% w/w to about 35% w/w, preferably in an amount of about 10% w/w to about 25% w/w, and more preferably in an amount of about 19% or about 20% based on the total weight of the final dosage form.
31. A final dosage form according to embodiment 29 or 30, wherein a filler is present in an amount of about 20 to about 40% w/w based on the total weight of the final dosage form.
32. A final dosage form according to embodiment 29, 30 or 31, wherein a disintegrating agent is present in an amount of about 5% w/w to about 10% w/w, preferably about 5 or about 6% based on the total weight of the final dosage form.
33. A final dosage form according to any one of embodiments 29-32 wherein the inert substrate is present in an amount of about 20% w/w to about 40% w/w, preferably about 30% based on the total weight of final dosage form.
34. A final dosage form according to any one of embodiments 29-33 wherein the binder is present in an amount of about 5% w/w to about 25% w/w, preferably about 8 to about 12% w/w, based on the total weight of the final dosage form.
35. A final dosage form according to any one of embodiments 29-34 wherein a lubricant is present in an amount of about 0.1 to about 2% w/w, preferably about 0.5% w/w to about 1.5% w/w based on the total weight of the final dosage form.
36. A final dosage form according to any one of embodiments 29-35 wherein a surfactant is present in an amount of about 0.1% w/w to about 2.5% w/w, preferably about 0.2% w/w to about 0.8% w/w based on the total weight of the final dosage form.
37. A final dosage form according to any one of embodiments 29-36 comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, in an amount of between about 0.5 mg to about 600 mg, e.g. about 5 mg to about 400 mg, e.g. about 10 mg to about 150 mg.
38. A final dosage form according to any one of embodiments 29-37 comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, in an amount of about 0.5 mg, about 5 mg, about 10 mg, about 15 mg, about 20 mg, about 25 mg, about 50 mg, about 100 mg, about 150 mg, about 200 mg, about 250 mg, about 300 mg, about 350 mg, about 400 mg, about 450 mg, about 500 mg, or of about 600 mg, preferably in an amount of about 10 mg, about 25 mg, about 50 mg and about 100 mg.
39. A process for preparing the pharmaceutical composition according to any one of embodiments 1-27, said process comprising the steps of:
   (i) Mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium, and
   (ii) Adding the said mixture (i) to the (a) inert substrate of the granule particles.
40. The process according to embodiment 39, wherein step (i) is performed in a wet milling chamber.
41. The process according to embodiment 39 or 40, wherein the liquid medium is an aqueous solution, e.g. purified water, preferably with a pH value between 5 and 8.
42. The process according to any one of embodiments 39-41, wherein the mixture of step (i) is dispersed onto the (a) inert substrate.
43. The process according to any one of embodiments 39-42, wherein the process further comprises preparing the final dosage form by blending the mixture resulting from step (ii) with at least one pharmaceutically acceptable excipient.
44. The process according to embodiment 43, wherein the final dosage form is encapsulated or tableted.
45. The process according to embodiment 44, wherein the final dosage form is tableted and the resulting tablet is further film coated.
46. A process for preparing a suspension comprising mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, with a liquid medium.
47. A suspension comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium.
48. The suspension according to embodiment 47 wherein the particle size of said suspension is less than 1000 nm, preferably less than 500 nm, more preferably less than 350 nm and most preferably less than 250 nm.
   The suspension according to embodiment 47 or 48, wherein the liquid medium is an aqueous solution, e.g. purified water, preferably with a pH value between 5 and 8, and more preferably between 5 and 6.
49. The suspension according to any one of embodiments 47 to 49, wherein N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, is present in an amount of about 10% to about 40% of the total weight of the suspension, preferably about 20% or about 25% of the total weight of the suspension.
50. The suspension according to embodiment 47 to 50, wherein the at least one binder is present in an amount of about 3% to about 15% of the total weight of the suspension.
51. The suspension according to embodiment 47 to 51, wherein the surfactant is present in an amount of about 0.05% to about 1% of the total weight of the suspension.
52. The pharmaceutical composition according to any one of embodiments 1-27, for use as a medicine, or a final dosage form according to any one of embodiments 29-37, for use as a medicine.
53. The pharmaceutical composition according to any one of embodiments 1-27, for use in the treatment or prevention of a disease or disorder mediated by BTK or ameliorated by inhibition of BTK or a final dosage form according to any one of embodiments 29-37 for use in the treatment or prevention of a disease or disorder mediated by BTK or ameliorated by inhibition of BTK.
54. The pharmaceutical composition for use according to embodiment 53 or 54, or the final dosage form according to embodiment 53 or 54 wherein the disease or disorder mediated by BTK or ameliorated by the inhibition of BTK is selected from autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis, myasthenia gravis, Sjögren's syndrome, autoimmune hemolytic anemia, anti-neutrophil cytoplasmic antibodies (ANCA)-associated vasculitides, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including but not limited to multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease. Preferably, the disease or disorder mediated by BTK or ameliorated by the inhibition of BTK is selected from rheumatoid arthritis; chronic urticaria, preferably chronic spontaneous urticaria; Sjögren's syndrome, multiple sclerosis or asthma.
55. Use of the pharmaceutical composition according to any one of embodiments 1-27 for the manufacture of a medicament for disease or disorder mediated by BTK or ameliorated by the inhibition of BTK is selected from autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis, myasthenia gravis, Sjögren's syndrome, autoimmune hemolytic anemia, anti-neutrophil cytoplasmic antibodies (ANCA)-associated vasculitides, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including but not limited to multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease. Preferably, the disease or disorder mediated by BTK or ameliorated by the inhibition of BTK is selected from rheumatoid arthritis; chronic urticaria, preferably chronic spontaneous urticaria; Sjögren's syndrome, multiple sclerosis or asthma.
56. A method of treating or preventing a disease or disorder mediated by BTK or ameliorated by the inhibition of BTK, comprising administering to a subject in need of such treatment or prevention, a pharmaceutical composition according to any one of embodiments 1-27, or a final dosage form according to any one of embodiments 29-37.
57. The method of embodiment 57 wherein the disease or disorder mediated by BTK or ameliorated by the inhibition of BTK is selected from autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis, myasthenia gravis, Sjögren's syndrome, autoimmune hemolytic anemia, anti-neutrophil cytoplasmic antibodies (ANCA)-associated vasculitides, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including but not limited to multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease. Preferably, the disease or disorder mediated by BTK or ameliorated by the inhibition of BTK is selected from rheumatoid arthritis; chronic urticaria, preferably chronic spontaneous urticaria; Sjögren's syndrome, multiple sclerosis or asthma.

### DETAILED DESCRIPTION OF THE INVENTION

The effective formulation of the BTK inhibitor N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, (referred herein as Compound (A)), prove difficult. For example, difficulties to formulate due to its strong pH dependent solubility issues, e.g. gelling tendencies at certain pH conditions, un-stabilities when exposed to some temperatures and/or UV light, poor dissolution rate (e.g. dispersability), low solubility, low exposure, and bioavailability issues were observed. Ultimately, those issues were affecting the manufacturing process of the pharmaceutical composition.

Surprisingly, it was found that those challenges can be overcome by preparing a pharmaceutical composition for oral administration comprising (a) an inert substrate, and (b) a mixture comprising a BTK inhibitor, and at least one binder. According to the present disclosure, the BTK inhibitor is N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, (referred herein as Compound (A)).

In one aspect the present invention provides a pharmaceutical composition for oral administration comprising a granule particle said granule particle comprising (a) an inert substrate, and (b) a mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, and at least one binder.

In another aspect of the present invention, Compound (A) is present as a pharmaceutically acceptable salt form. In a preferred aspect of the invention, Compound (A) is present in its free form, e.g. Compound (A) is present in its anhydrous form. In particular, Compound (A) is present as a crystalline form (A) which is described in WO2020/234779 filed on May 20, 2020 (attorney docket number PAT058512) In yet another embodiment, the crystalline form of Compound (A) is substantially phase pure.

According to the present invention, the pharmaceutical composition comprises an (a) inert substrate on which the (b) mixture comprising Compound (A), and at least one binder, is added. The inert substrate comprises a material that does not chemically react to the (b) mixture comprising Compound (A), and at least one binder. The (a) inert substance is, for example, a pharmaceutically acceptable excipient known in the art not to interact chemically or physically with the active substance. Optionally, the (a) inert substance can also be coated with a layer to protect the (a) inert substance from any unwanted chemical or physical interaction that may happen during the formulation process. In such instance, the term “inert substrate” is used interchangeably with the term “carrier particles”. The (a) inert substance may comprise a material, which is selected from the group consisting of lactose, microcrystalline cellulose, mannitol, sucrose, starch, granulated hydrophilic fumed silica, sugar beads (Kayaert et al., J. Pharm. Pharmacol. 2011, 63, 1446-1453), polymer films (Sievens-Figueroa et al., Int. J. Pharm. 2012, 423, 496-508), or mixtures thereof. Preferably, the material is selected from the group consisting of lactose, mannitol, or mixtures thereof. More preferably, the material is mannitol, such as mannitol SD, mannitol SD100, or mannitol SD200.

The granule particle size is measured, for example, by laser diffraction methodology (e.g. particle size distribution (PSD)) using methods and instruments known to the skilled person in the art.

Suitable binders can be selected, for example, from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose (e.g. sodium cellulose gum, cellulose gum), methyl cellulose (e.g. cellulose methyl ether, Tylose), hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, vitamin E polyethylene glycol succinate, or a mixture thereof. Preferably, the binder is polyvinylpyrrolidone-vinyl acetate copolymer (also known as copovidone).

The at least one binder present in the (b) mixture can be present in an amount from about 25% w/w to about 100% w/w based on the weight of Compound (A). The above-mentioned ranges apply for all the binders as listed above. Preferably, the binder is polyvinylpyrrolidone-vinyl acetate copolymer and is present in an amount from about 25% w/w to about 100% w/w based on the weight of Compound (A). In a preferred embodiment, the binder (preferably copovidone) is present in the (b) mixture in an amount of about 50% or about 100% w/w based on the weight of compound (A). In yet another preferred embodiment, the weight ratio of compound (A) and the binder in the (b) mixture is in a range from about [3:1] to about [1:3]; e.g. about [3:1], about [2:1], about [1:1], about [1:2] or about [1:3], preferably [2:1]. More preferably, the weight ratio of compound (A) and the binder in the (b) mixture is about [1:1]. In yet another embodiment, the weight ratio of compound (A) and the binder in the pharmaceutical composition is about [3:1] about [2:1] or about [1:1], most preferably [2:1].

In another aspect, the present invention also provides a pharmaceutical composition (e.g. for oral administration), wherein the (b) mixture optionally further comprises a surfactant. According to the present invention, the pharmaceutical composition (e.g. for oral administration) comprises an (a) inert substrate on which the (b) mixture comprising Compound (A), at least one binder, and optionally a surfactant, is added. Suitable surfactants can be selected, for example, from the group consisting of sodium lauryl sulfate (SLS), potassium lauryl sulfate, ammonium lauryl sulfate, sodium lauryl ether sulfate, polysorbates, perfluorobutanesulfonate, dioctyl sulfosuccinate, or a mixture thereof. Preferably, the surfactant is sodium lauryl sulfate (SLS).

The surfactant when present in the (b) mixture can be present in an amount of about 1% w/w to about 10% w/w based on the weight of Compound (A). The above-mentioned ranges apply for all the surfactants as listed above. Preferably, the surfactant is sodium lauryl sulfate (SLS) and is present in an amount of about 1% w/w to about 10% w/w based on the weight of Compound (A), preferably in an amount of 2 to 6% w/w based on the weight of compound (A), more preferably in an amount of about 4% w/w or about 5% w/w based on the weight of compound (A). In accordance with the aspect of the present invention, when the surfactant is present, the weight ratio of Compound (A), at least one binder, and the surfactant in the (b) mixture is about [3:1:1], or about [3:1:0.5], or about [3:1:0.1], or about [2:1:1], or about [2:1:0.5], or about [2:1:0.1], or about [2:1:0.08], or about [2 1:0.05], or about [2:1:0.04], or about [2:1:0.03], or about [2:1:0.02], or about [1:1:0.5], or about [1:1:0.1], or about [1:1:0.07], or about [1:1:0.05], or about [1:1:0.04], or about [1:1:0.02]. Preferably, the ratio is about [2:1:1], or about [2:1:0.5], or about [2:1:0.1], or about [2:1:0.08], or about [2:1:0.05], or about [2:1:0.04], or about [2:1:0.03], or about [2:1:0.02], or about [1:1:0.5], or about [1:1:0.1], or about [1:1:0.07], or about [1:1:0.05], or about [1:1:0.04], or about [1:1:0.02], or about [1:3:0.1], or about [1:3:0.2], or about 1;1.5:0.25]. More preferably, the ratio is about [2:1:1], or about [2:1:0.08], or about [2:1:0.5], or about [2:1:0.1], or about [2:1:0.05], or about [2:1:0.04], or about [2:1:0.03], or about [2:1:0.02]. In one embodiment, when the surfactant is present, the weight ratio of Compound (A), at least one binder, and the surfactant in the (b) mixture is the ratio is about [2:1:0.08]. In a particular embodiment, the surfactant is SLS and the binder is copovidone, and the weight ratio of compound (A), copovidone and SLS in the (b) mixture is about [2:1:1], or about [2:1:0.08], or about [2:1:0.5], or about [2:1:0.1], or about [2:1:0.05], or about [2:1:0.04], or about [2:1:0.03], or about [2:1:0.02]; more preferably about [2:1:0.08].

In another embodiment, when the surfactant is present, the weight ratio of Compound (A), the at least one binder, and the surfactant in the pharmaceutical composition is about [2:1:1], or about [2:1:0.08], or about [2:1:0.5], or about [2:1:0.1], or about [2:1:0.05], or about [2:1:0.04], or about [2:1:0.03], or about [2:1:0.02]. in a further aspect, when the surfactant is present, the weight ratio of Compound (A), at least one binder, and the surfactant in the pharmaceutical composition is about [2:1:0.08]. In a particular aspect of this embodiment, the surfactant is SLS and the binder is copovidone, and the weight ratio of compound (A), copovidone and SLS in the pharmaceutical composition is about [2:1:1], or about [2:1:0.08], or about [2:1:0.5], or about [2:1:0.1], or about [2:1:0.08], or about [2:1:0.05], or about [2:1:0.04], or about [2:1:0.03], or about [2:1:0.02]. more preferably about [2:1:0.08].

In accordance with the aspect of the present invention, the (b) mixture comprising Compound (A), at least one binder, and optionally a surfactant, is pre-mixed together. The (b) mixture can be added to a liquid medium in which it is essentially insoluble to form a pre-mix. The liquid medium can be for example aqueous or non-aqueous in nature. Preferably, the liquid medium is an aqueous solution, for example water. According to the aspect of the present invention, the (b) mixture is in the form of a suspension or a dispersion, more preferably a suspension.

Compound (A) can be present in the liquid medium in an amount of about 5% w/w to about 40% w/w based on the total combined weight of the pre-mix, preferably, in an amount of about 10% w/w, or in an amount of about 15% w/w, or in an amount of about 20% w/w, or in an amount of about 25% w/w, or in an amount of about 30% w/w, more preferably about 20% w/w based on weight of the pre-mix.

At least one binder can be present in the liquid medium in an amount of about 3% w/w to about 15% w/w based on weight of the pre-mix; preferably in an amount of about 4% w/w, or about 6% w/w, or about 8% w/w or about 10% w/w, more preferably about 4% w/w based on the weight of the pre-mix.

The surfactant when present, is present in the liquid medium in an amount of about 0.05% to about 1% based on the weight of the pre-mix, preferably about 0.1%, or about 0.5%, or about 0.75%, more preferably about 0.1% w/w based on the weight of the pre-mix.

According to the present invention, the pre-mix can be used directly or can be subjected to mechanical means to reduce the average particle size to less than 1000 nm. The particle size is measured, for example, by laser diffraction methodology (e.g. particle size distribution (PSD)) using methods and instruments known to the skilled person in the art. Preferably, the particle size as measured by PCS is less than 500 nm, more preferably less than 350 nm and most preferably less than 250 nm. In one embodiment, the particle size of the suspension as measured by PCS is between about 50 nm to about 1000 nm, or between about 50 nm to 500 nm, or between about 50 nm to about 350 nm, or between about 100 nm to 170 nm, e.g. the particle size is about 50 nm, or about 70 nm, or about 90 nm, or about 100 nm, or about 110 nm, or about 120 nm, or about 130 nm, or about 140 nm, or about 150 nm, or about 160 nm, or about 170 nm, or about 180 nm, or about 190 nm, or about 200 nm, or about 230 nm or about 250 nm, or about 280 nm, or about 300 nm, or about 320 nm, or about 350 nm, or about 370 nm, or about 400 nm, or about 450 nm, or about 500 nm. More preferably, the particle size is between about 100 nm to about 350 nm, or between about 110 nm to about 180 nm, or between about 250 nm to about 350 nm. The particles formed are stabilized by the presence of the binder in the pre-mix, as defined herein, which is able to maintain the particles at the desired size, in a stable state.

In accordance with the present invention, the (b) mixture, as defined herein, comprising Compound (A), at least one binder, and optionally a surfactant, can be added onto the (a) inert substrate using different techniques known in the art, as described herein. Preferably, the (b) mixture, as defined herein, comprising Compound (A), at least one binder, and optionally a surfactant, is dispersed onto the (a) inert substrate. In another preferred aspect, the (a) inert substrate is coated with the (b) mixture comprising Compound (A), at least one binder, and a surfactant. In another preferred aspect, as defined herein, the (b) mixture comprising Compound (A), at least one binder, and optionally a surfactant, is a suspension and is preferably dispersed or coated onto the (a) inert core as discrete particles, thus, providing a large surface area for instant dissolution despite the poor solubility of the drug.

Another aspect of the present invention provides a suspension comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, (referred herein as Compound (A)), at least one binder, and optionally a surfactant, in a liquid medium such as an aqueous solution (e.g. purified water, preferably with a pH value between 5 and 8, and more preferably between 5 and 6). According to the present invention, the particle size of said suspension as measured by PCS is less than 1000 nm, preferably less than 500 nm, and more preferably less than 350 nm and most preferably less than 250 nm, as defined herein. In particular, the average particle size of said suspension as measured by PCS is between about 50 nm to about 1000 nm, or between about 50 nm to 500 nm, or between about 50 nm to about 350 nm, or between about 100 nm to 170 nm, e.g. the particle size is about 50 nm, or about 70 nm, or about 90 nm, or about 100 nm, or about 110 nm, or about 120 nm, or about 130 nm, or about 140 nm, or about 150 nm, or about 160 nm, or about 170 nm, or about 180 nm, or about 190 nm, or about 200 nm, or about 230 nm, or about 250 nm, or about 280 nm, or about 300 nm, or about 320 nm, or about 350 nm, or about 370 nm, or about 400 nm, or about 450 nm, or about 500 nm. More preferably, the particle size is between about 100 nm to about 350 nm, or between about 110 nm to about 180 nm, or between about 250 nm to about 350 nm.

Another aspect of the present invention provides a dispersible solution comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, (referred herein as Compound (A)), at least one binder, and optionally a surfactant, in a liquid medium such as an aqueous solution (e.g. purified water, preferably with a pH value between 5 and 8, and more preferably between 5 and 6).

According to the present invention, the pharmaceutical composition is prepared by mixing together about 0.5 mg to about 600 mg of Compound (A), with at least one binder, and optionally a surfactant. Preferably, the pharmaceutical composition is prepared by mixing together about 5 mg to about 400 mg of Compound (A), with at least one binder, and optionally a surfactant. More preferably, the pharmaceutical composition is prepared by mixing about 10 mg to about 150 mg of Compound (A), with at least one binder, and optionally a surfactant. The pharmaceutical composition (e.g. for oral administration), as disclosed herein, can comprise a mixture of 10 mg of Compound (A), with at least one binder and optionally a surfactant. The pharmaceutical composition can also comprise a mixture of 15 mg of Compound (A), with at least one binder, and optionally a surfactant. In another example, the pharmaceutical composition (e.g. for oral administration) can also be prepared with 20 mg of Compound (A), and at least one binder, and optionally a surfactant. In another example, the pharmaceutical composition (e.g. for oral administration) can also comprise, for example, 25 mg of Compound (A), at least one binder, and optionally a surfactant. In another example, the pharmaceutical composition is prepared by mixing together 50 mg of Compound (A), with at least one binder, and optionally a surfactant. In another example, the pharmaceutical composition is prepared by mixing together 100 mg of Compound (A), with at least one binder, and optionally a surfactant. In another example, the pharmaceutical composition (e.g. for oral administration) can also be prepared by mixing together 150 mg of Compound (A), with at least one binder, and optionally a surfactant. In another example, the pharmaceutical composition is prepared by mixing together 200 mg of Compound (A), with at least one binder, and optionally a surfactant. In another example, the pharmaceutical composition (e.g. for oral administration) can also be prepared by mixing together 250 mg of Compound (A), with at least one binder, and optionally a surfactant. In another example, the pharmaceutical composition is prepared by mixing together 300 mg of Compound (A), with at least one binder, and optionally a surfactant. In another example, the pharmaceutical composition (e.g. for oral administration) can also be prepared by mixing together 350 mg of Compound (A), with at least one binder, and optionally a surfactant. In another example, the pharmaceutical composition is prepared by mixing together 400 mg of Compound (A), with at least one binder, and optionally a surfactant. In another example, the pharmaceutical composition (e.g. for oral administration) can also be prepared by mixing together 450 mg of Compound (A), with at least one binder, and optionally a surfactant. In another example, the pharmaceutical composition is prepared by mixing together 500 mg of Compound (A), with at least one binder, and optionally a surfactant. In another example, the pharmaceutical composition (e.g. for oral administration) can also be prepared by mixing together 600 mg of Compound (A), with at least one binder, and optionally a surfactant.

In accordance with the aspect of the present invention, the granule particles, as defined herein, can optionally comprises an outer seal coating layer. The outer seal coating layer comprises a material that does not chemically react to the (b) mixture, as defined herein, and protects the (b) mixture from any unwanted chemical or physical interaction that may happen during the formulation process, e.g. with additives, pharmaceutically acceptable excipients, or any further active pharmaceutical ingredient. The outer seal coating layer can also provide an additional barrier for taste masking, and also for gastric or stomach release while allowing for enteric or intestinal release. If present, the outer seal coating layer can be selected from, for example, but not limited to, hydroxypropyl methyl cellulose, magnesium stearate, polyvinyl pyrrolidone, hydroxypropyl cellulose, carboxymethyl cellulose, methyl cellulose, hydroxyethyl cellulose, carboxyethyl cellulose, carboxymethylhydroxyethyl cellulose, polyethylene glycol, polyvinylalcohol, cellulose acetate phthalates (CAP), cellulose acetate trimellitates (CAT), hydroxypropyl methyl cellulose phthalates (HPMCP), hydroxypropyl methyl cellulose acetate succinate (HPMCAS), polyvinyl acetate phthalate (PVAP), methyl methacrylate-methacrylic acid copolymers, cellulose acetate succinate, fatty acids, waxes, shellac, sodium alginate, or mixtures thereof.

In one embodiment, the invention provides a pharmaceutical composition as defined above wherein the particle size of the drug substance (i.e. compound (A)) is less than 1000 nm. Preferably, the particle size of compound (A) as measured by PCS is less than 500 nm, more preferably less than 350 nm and most preferably less than 250 nm. In one embodiment, the particle size of compound (A) as measured by PCS is between about 50 nm to about 1000 nm, or between about 50 nm to 500 nm, or between about 50 nm to about 350 nm, or between about 100 nm to 170 nm, e.g. the particle size is about 50 nm, or about 70 nm, or about 90 nm, or about 100 nm, or about 110 nm, or about 120 nm, or about 130 nm, or about 140 nm, or about 150 nm, or about 160 nm, or about 170 nm, or about 180 nm, or about 190 nm, or about 200 nm, or about 230 nm or about 250 nm, or about 280 nm, or about 300 nm, or about 320 nm, or about 350 nm, or about 370 nm, or about 400 nm, or about 450 nm, or about 500 nm. More preferably, the particle size of compound (A) is between about 100 nm to about 350 nm, or between about 110 nm to about 180 nm, or between about 250 nm to about 350 nm.

A further aspect of the present invention provides a process for preparing the pharmaceutical composition (e.g. for oral administration), as defined herein, said process comprising the steps of:
(i) Mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium, and
(ii) Adding the said mixture (i) to the (a) inert substrate of the carrier particles.

Another aspect of the present invention provides a process for preparing the pharmaceutical composition (e.g. for oral administration), as defined herein, said process comprising the steps of:
(iii) Mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium, wherein the liquid medium is an aqueous solution or non-aqueous solution, and
(iv) Adding the said mixture (i) to the (a) inert substrate of the carrier particles.

Another aspect of the present invention relates to a process for preparing the pharmaceutical composition (e.g. for oral administration), as defined herein, said process comprising the steps of:
(i) Mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in an aqueous solution, wherein the aqueous solution is water, and
(ii) Adding the said mixture (i) to the (a) inert substrate of the carrier particles, wherein the BTK inhibitor, such as Compound (A), is present in an amount of about 0.5 mg to about 600 mg, or about 5 mg to about 400 mg, or about 10 mg to about 150 mg, as defined herein.

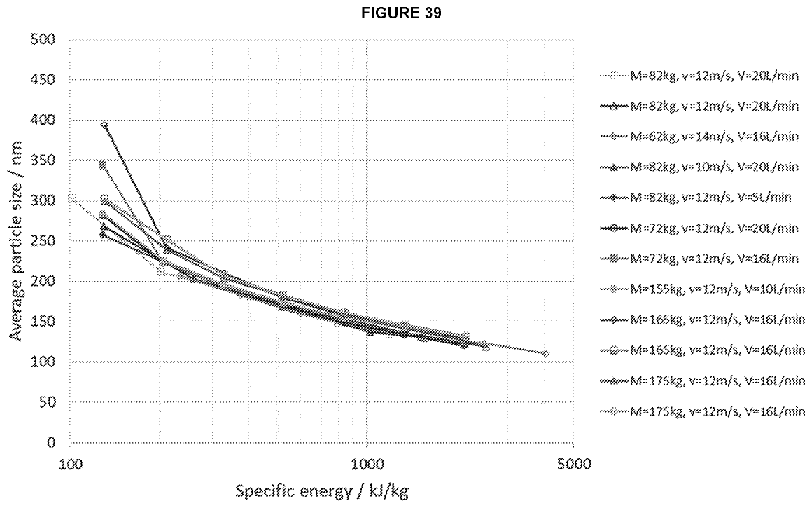

As mentioned herein above the (b) mixture can be added to a liquid medium (e.g. an aqueous solution) in which it is essentially insoluble to form a pre-mix. The pre-mix can be dispersed or suspended in the liquid medium using suitable agitation, until a homogenous dispersion or suspension is observed in which there are no large agglomerates visible in the naked eye. Mechanical means that can be used to reduce the particle size of Compound (A) are any mechanical means known to the skilled person in the art. Preferably, the mechanical means used to reduce the particle size of the (b) mixture (or pre-mix) comprising Compound (A) is a milling mean performed in a milling chamber. Suitable milling techniques include, for example, ball milling, wet milling, media milling, wet media milling, stirred milling, stirred media milling, wet stirred media milling, agitator milling, agitator media milling, wet agitator media milling, bead milling, agitator bead milling, wet agitator bead milling, and high pressure homogenization. Preferably, the nano-sized particles are prepared using a milling technique selected from wet milling, media milling, wet media milling or high-pressure homogenization More preferably, the milling technique is wet milling, media milling, and wet media milling. Specifically, the nano-sized particles are prepared using a wet media milling technique. Thus, in accordance with the present invention, step (i) of the process, as defined herein, is performed in a milling chamber, in particular in a wet milling chamber. The pH of the pre-mix in the milling chamber is about pH=5 and pH=8, preferably the pH in the milling chamber is about 6. The process is performed with process parameters resulting in minimum specific energy introduced into the suspension of 200 kJ/kg, and a suspension temperature at the outlet of the grinding chamber of up to 35° C. temperature. More preferably, the process is performed with higher specific energies of above 200 kJ/kg, and lower suspension temperatures at the outlet of the grinding chamber of below 35° C. temperature. Specific energies are calculated according to Kwade (Kwade, Powder Technology 1999, 105, 14-20, and Kwade, Chemical Engineering and Technology 2003, 26, 199-205). This relationship was investigated for different batch size, e.g. from about 62 to 175 kg, rotor tip speed, e.g. from 10 to 14 m/s, and liquid flow rate, e.g. from 5 to 20 L/min. FIG. 39 shows the established relationship between average particle size and specific energy for differently manufactured batches, considering various batch sizes and process parameter settings for rotor tip speed and suspension flow rate. Particle size of Compound (A) was reasonably controlled by parameter specific energy, despite the difference in batch size, rotor tip speed and suspension flow rate investigated. The process was performed with process parameters resulting in minimum specific energy introduced into the suspension of about 200 kJ/kg, and a suspension temperature at the outlet of the grinding chamber of up to 35° C. Preferably, the process was performed with higher specific energies of above 300 kJ/kg, and a suspension temperature at the outlet of the grinding chamber of up to 32° C. Most preferably, the process was performed with specific energies of above 600 kJ/kg, and a suspension temperature at the outlet of the grinding chamber between 16 and 32° C.

Therefore, one aspect of the invention is to provide a suspension comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium. In one aspect, the particle size of said suspension is less than 1000 nm, preferably less than 500 nm and more preferably less than 350 nm and most preferably less than 250 nm. In another aspect, the liquid medium of the said suspension is an aqueous solution, e.g. purified water, preferably with a pH value between 5 and 8, and more preferably between 5 and 6.

In another aspect, the suspension as described above comprises compound (A), or a pharmaceutically acceptable salt thereof, or a free form thereof, wherein compound (A), or a pharmaceutically acceptable salt thereof, or a free form thereof, is present in an amount of about 10% to about 40% of the total weight of the suspension, preferably about 20% or about 25% of the total weight of the suspension.

In yet a further aspect, the invention provides a suspension as described above wherein the at least one binder (preferably copovidone) is present in an amount of about 3% to about 15% of the total weight of the suspension.

In yet a further aspect, the invention provides a suspension as defined above, wherein the surfactant (preferably SLS) is present in an amount of about 0.05% to about 1% of the total weight of the suspension.

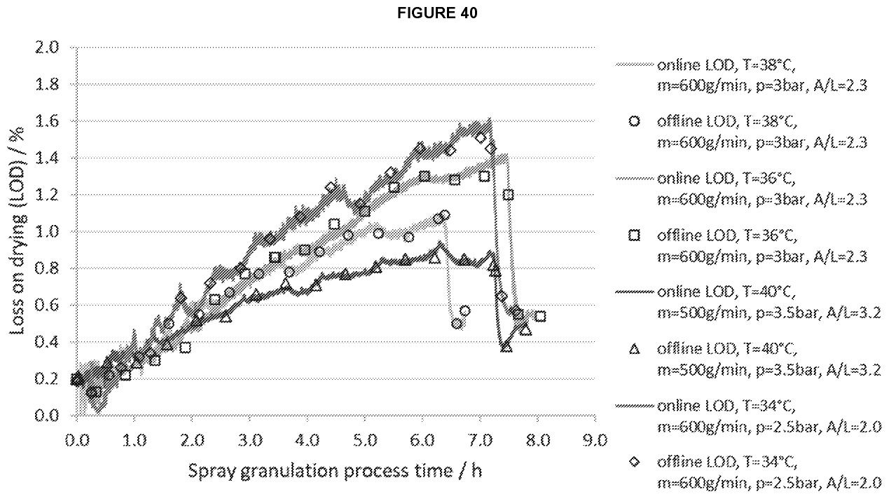

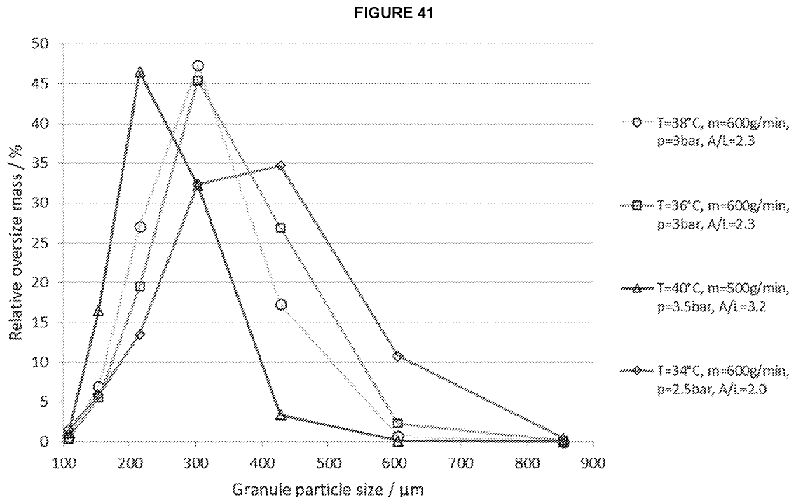

In accordance with the present invention, the process for preparing the pharmaceutical composition (e.g. for oral administration), as defined herein, comprises adding the (b) mixture from step (i) to the (a) inert substrate of the carrier particles, as defined herein. The (b) mixture can be added using different techniques known in the art, such as, for example, spray drying, spray granulation, spray layering, spray dispersing, spray coating, fluid bed drying, fluid bed coating, fluid bed spray granulation, granulators with spray nozzles, or a combination of those spraying techniques thereof. According to the present invention, the coating or spraying can be done, for example, from above the carrier particle (e.g. top spraying or top coating), underneath the carrier particle (e.g. bottom spraying or bottom coating), simultaneously or subsequently from both direction. According to the present invention, top spraying or top coating is preferred. Preferably, the (b) mixture, as defined herein, wherein the (a) inert substrate is coated with the (b) mixture. More preferably, the mixture of the process step (i) is dispersed onto the (a) inert substrate. Specifically, the (b) mixture is added using, for example, spray drying, spray granulation, fluid bed spray granulation, or a combination of those spraying techniques thereof. The liquid medium, e.g. purified water, is evaporated keeping the product (compound (A)) temperature between about 30° C. and about 45° C. Preferably, the product temperature is of about 36° C. to about 44° C. More preferably, at a temperature of about 36° C. to about 40° C. During the spray granulation process, the spray rate and atomization air pressure are parameters which determine the droplet size of the spray liquid, when sprayed. Those parameters are dependent on the nozzle geometry. Each nozzle is characterized by a factor which is the air consumption at a specific atomization air pressure. This factor is provided from the nozzle manufacturer typically in an air consumption chart. This value and the used spray rate was used to calculate the air mass to liquid mass ratio applied during the spray process. The granulation process is carried out using spray rate and atomization air pressure resulting in a range for “air mass to liquid mass flow ratio” of about 1.1 to about 3.2, e.g. about 1.1 to about 2.3. The ratio of air mass to liquid mass flow between is important about 1.1 to about 3.2 as it controls the droplet size distribution of the liquid after atomization. The droplet size increases with the decreasing air to liquid ratio which results in granules less optimal for later tablet compression, uniformity of blend and segregation risk. Loss on drying (LOD) of the granules is a well-accepted surrogate to quantitatively describe the complex relationship of material and process parameters during spray granulation processing, considering e.g. material parameter spray liquid and process parameters spray rate, air flow rate and inlet air temperature (Ochsenbein D. R. et al., Int. J. Pharm. X1 (2019) 100028, Lyngberg O. et al., Applications of Modeling in Oral Solid Dosage Form Development and Manufacturing, In: Process Simulation and Data Modeling in Solid Oral Drug Development and Manufacture, Ierapetritou M. G. and Ramachandran R. (Editors), Humana Press (2016) 1-42). Loss on drying (LOD) trajectories were experimentally established as a characteristic surrogate for the most preferable process conditions (i.e. process conditions wherein product temperature is of about 34 to about 40° C. and air to liquid ratio is of about 2.0 to about 3.2). FIG. 40 shows the LOD trajectories for the most preferable process conditions as defined above. The higher and lower LOD trajectories demonstrate the range of the most preferable process conditions for rather wet process conditions (higher LOD trajectory) and rather dry process conditions (lower LOD trajectory). The corresponding product granule particle size distributions are shown in FIG. 41. The rather wet process conditions (higher LOD trajectory) results in coarser granule particle size distribution, and the rather dry process conditions (lower LOD trajectory) results in finer granule particle size distribution. The granule particle size distribution was reasonably controlled by the most preferable process conditions as defined above as expressed by the LOD trajectories. Process conditions beyond the higher and lower LOD trajectories resulted in granules with less optimal properties for tablet compression and uniformity of blend.

In a further aspect of the present invention provides a process for preparing a suspension comprising mixing the (b) mixture as defined herein, in a liquid medium, as defined herein. Thus, another aspect of the present invention relates to a process for preparing the pharmaceutical composition (e.g. for oral administration), as defined herein, said process comprising the steps of:
(i) Preparing a suspension by mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium, wherein the liquid medium is an aqueous solution, e.g. purified water, preferably with a pH value between 5 and 8, and more preferably between 5 and 6, and
(ii) Adding the suspension from step (i) to the (a) inert substrate of the carrier particles. In one aspect of the above process, the suspension has an average particle size as measured by PCS of less than 1000 nm. Preferably, the particle size of the suspension as measured by PCS is less than 500 nm, more preferably less than 350 nm and most preferably less than 250 nm. In one embodiment, the particle size of the suspension as measured by PCS is between about 50 nm to about 1000 nm, or between about 50 nm to 500 nm, or between about 50 nm to about 350 nm, or between about 100 nm to 170 nm, e.g. the particle size is about 50 nm, or about 70 nm, or about 90 nm, or about 100 nm, or about 110 nm, or about 120 nm, or about 130 nm, or about 140 nm, or about 150 nm, or about 160 nm, or about 170 nm, or about 180 nm, or about 190 nm, or about 200 nm, or about 230 nm or about 250 nm, or about 280 nm, or about 300 nm, or about 320 nm, or about 350 nm, or about 370 nm, or about 400 nm, or about 450 nm, or about 500 nm. More preferably, the particle size is between about 100 nm to about 350 nm, or in between about 110 nm to about 180 nm, or between about 250 nm to about 350 nm.

In another aspect, the present invention provides a process for preparing a dispersion comprising mixing the (b) mixture as defined herein, with a liquid medium, as defined herein. Thus, another aspect of the present invention relates to a process for preparing the pharmaceutical composition (e.g. for oral administration), as defined herein, said process comprising the steps of:
(i) Preparing a dispersion by mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium, wherein the liquid medium is an aqueous solution, e.g. e.g. purified water, preferably with a pH value between 5 and 8, and more preferably between 5 and 6, and
(ii) Adding said dispersion from step (i) to the (a) inert substrate of the carrier particles. In one aspect of the process above, the suspension has an average particle size as measured by PCS of less than 1000 nm. Preferably, the particle size is between about 50 nm to about 1000 nm, or between about 50 nm to 500 nm, or between about 50 nm to about 350 nm, or between about 100 nm to 170 nm, e.g. the particle size is about 50 nm, or about 70 nm, or about 90 nm, or about 100 nm, or about 110 nm, or about 120 nm, or about 130 nm, or about 140 nm, or about 150 nm, or about 160 nm, or about 170 nm, or about 180 nm, or about 190 nm, or about 200 nm, or about 230 nm or about 250 nm, or about 280 nm, or about 300 nm, or about 320 nm, or about 350 nm, or about 370 nm, or about 400 nm, or about 450 nm, or about 500 nm. More preferably, the particle size is between about 110 nm to about 350 nm, or between about 110 nm to about 160 nm, or between about 250 nm to about 350 nm.

In a further aspect of the present invention relates to a process for preparing the pharmaceutical composition (e.g. for oral administration), as defined herein, said process further comprises preparing the final dosage form by blending the mixture resulting from step (ii) with an external phase, said external phase comprising at least one pharmaceutically acceptable salt thereof. For example, the external phase as defined herein, can be added to prevent chemical-physical interactions between the particles and any other active or non-active substance that may be used in the preparation of the final dosage form. Additional advantage of the external phase is to provide acceptable rate of dissolution, acceptable disintegration time, better processability and tablettability properties such as tablet tensile strength.

Another aspect of the invention also provides the process for preparing the unit dosage form (e.g. for oral administration) comprising the steps of:
(i) Mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder (e.g. polyvinylpyrrolidone-vinyl acetate copolymer), and optionally a surfactant (e.g. sodium lauryl sulfate (SLS)), in a liquid medium such as an aqueous solution (e.g. purified water, preferably with a pH value between 5 and 8, and more preferably between 5 and 6), in a wet milling chamber, wherein the average particle size of Compound (A) in the (b) mixture is less than 1000 nm, preferably less than 500 nm and more preferably less than 350 nm and most preferably less than 250 nm (the particle size is e.g. between about 100 nm to about 350 nm, or between about 110 nm to 180 nm, as disclosed herein)
(ii) Adding the said mixture (i) to the (a) inert substrate of the carrier particles, and
(iii) Blending the mixture resulting from step (ii) with at least one pharmaceutically acceptable excipient, to obtain the final dosage form, wherein the BTK inhibitor, such as Compound (A), is present in an amount of about 0.5 mg to about 600 mg, or about 5 mg to about 400 mg, or about 10 mg to about 150 mg (as defined herein).

Another aspect of the present invention provides the process for preparing the pharmaceutical composition, wherein the process, for example, follows the below process flowchart.

Another aspect the present invention provides for a process, as defined herein, wherein the final dosage form is encapsulated or tableted. When the final dosage is a tablet, the tablet may be film coated.

Another aspect of the present invention provides for a process further comprising preparing the final dosage form by mixing the carrier particles with at least one pharmaceutically acceptable excipient (external phase). The carrier particles can be transformed into a final dosage form (e.g. tablet, capsule) by, for example, granulation, freeze-drying, or spray drying, using at least one pharmaceutically acceptable excipient and/or matrix formers. Suitable pharmaceutically acceptable excipient can be selected, for example, from the group consisting of lactose, mannitol (such as mannitol DC), microcrystalline cellulose (e.g. Avicel PH101®, Avicel PH102®), dicalcium phosphate, polyvinyl pyrrolidone, hydroxypropyl methylcellulose, croscarmellose sodium, polyvinylpyrrolidone-vinyl acetate copolymer (e.g. crospovidone), sodium starch glycolate, colloidal silicon dioxide, magnesium stearate, sodium bicarbonate, sodium stearyl fumarate, or mixtures thereof. Preferably, the excipient can be selected from the group consisting of mannitol (such as mannitol DC), croscarmellose sodium, colloidal silicon dioxide, magnesium stearate, sodium bicarbonate, or mixtures thereof. The at least one pharmaceutically acceptable excipient is selected to provide a formulation with a good disintegration and dispersion of Compound (A), thus reducing its gelling behavior.

The pharmaceutical composition, as disclosed herein, is intended to be administered orally to humans and animals in unit dosage forms, or multiple-dosage forms, such as, for example, a capsule, a caplet, a powder, pellets, granules, a tablet, a minitablet, (up to 3 mm or up to 5 mm) a sachet, a pouch, or a stick pack. Preferably, the unit dosage form, or multi-dosage form, for example, is a capsule, a tablet, a sachet, a pouch, or a stick pack. More preferably, the pharmaceutical composition is in the form of a capsule, or a tablet. This can be achieved by mixing the pharmaceutical composition, as defined herein, with fillers (or also referred to as diluents), lubricants, glidants, disintegrants, and/or absorbents, colorants, flavours and sweeteners.

Capsules comprising the pharmaceutical composition of the invention, as defined herein, can be prepared using techniques known in the art. Suitable capsules can be selected from hard shell capsule, hard gelatin capsule, soft gelatin capsule, soft shell capsule, plant-based shell capsule, hypromellose (HPMC) based capsule, or mixtures thereof. The pharmaceutical composition, as described herein, can be presented in a hard gelatin capsule, a hard shell capsule, or a hard plant shell capsule, hypromellose (HPMC) capsule wherein the pharmaceutical composition is further mixed with an inert solid diluent, for example, calcium carbonate, calcium phosphate, magnesium stearate, sodium bicarbonate, or cellulose-based excipient (e.g. microcrystalline cellulose). The hard gelatin capsules are made of two-piece outer gelatin shells referred to as the body and the cap. The shell may comprise vegetal or animal gelatin (e.g. pork, beef, or fish based gelatin), water, one or more plasticizers, and possibly some preservatives. The capsule may hold a dry mixture, in the form of a powder, very small pellets, or particles, comprising a BTK inhibitor, such as Compound (A), at least one binder, and optionally surfactants and/or other excipients. The shell may be transparent, opaque, colored, or flavored. The capsules containing the particles can be coated by techniques well known in the art with enteric- and/or gastric-resistant or delayed-release coating materials, to achieve, for example, greater stability in the gastrointestinal tract, or to achieve the desired rate of release. Hard gelatin capsules of any size (e.g. size 000 to 5) can be prepared.

Tablets comprising the pharmaceutical composition of the invention, as defined herein, can be prepared using techniques known in the art. Suitable tablets may contain the particles in admixture with non-toxic pharmaceutical, which are suitable for the manufacture of tablets. These excipients are, for example, inert diluents (or otherwise referred as fillers), such as calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof; disintegrating agents (or also referred to as disintegrants), for example, croscarmellose sodium, crospovidone, sodium starch glycolate, corn starch, or alginic acid, or mixture thereof; gliding agents (or also referred to as glidants), for example, fumed silica (e.g. Aerosil®, Aeroperl®); binding agents (or also referred to as binders) (e.g. for example, methyl cellulose, carboxymethyl cellulose, polyvinyl pyrrolidone, starch, gelatin, or acacia), or mixture thereof; and lubricating agents (or also referred to as lubricants), for example magnesium stearate, sodium stearyl fumarate, stearic acid or talc or mixture thereof. The mixture of the particles in admixture with non-toxic pharmaceutical can be mixed using numerous known methods, such as, for example, mixing in a free-ball, or tumble blending. The mixture of the particles in admixture with non-toxic pharmaceutical can be compressed into a tablet using tableting techniques known in the art, such as, for example, a single punch press, a double punch press, a rotary tablet press, or a compaction on a roller compaction equipment. The compression force applied to form the tablet can be any suitable compression force that allows obtaining a tablet, for example, the compression applied can be from 0.5 to 60 kN, or from 1 to 50 kN, or from 5 to 45 kN. Preferably, the compression force is from 5 to 25 kN. The tablets or granules can be uncoated or coated by known techniques to delay disintegration and absorption in the gastrointestinal tract and thereby provide a sustained action over a longer period. For example, tablets can be coated with a suitable polymer or a conventional coating material to achieve, for example, greater stability in the gastrointestinal tract, or to achieve the desired rate of release, for example the tablet can be coated with hypromellose (HPMC), magnesium stearate, polyethylene glycol (PEG), polyvinyl alcohol (PVA), Opadry®, Opadry II®, or mixtures thereof. For example, a time delay material such as glyceryl monostearate or glyceryl distearate can be employed. Tablets of any shape or size can be prepared, and they can be opaque, coloured, or flavoured. Specifically, the pharmaceutical composition as disclosed herein, is in the form of a filmed coated tablet.

The BTK inhibitor, such as N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, (referred to herein as Compound (A)), is present in the pharmaceutical composition in an amount sufficient to exert a therapeutically useful effect in the absence of undesirable side effects on the patient treated. Each unit dose contains a predetermined amount of the Compound (A), sufficient to produce the desired therapeutic effect. Each unit dose as disclosed herein, are suitable for human and animal subjects, are packaged individually and may be administered in fractions or multiples thereof. A multiple-dose form is a plurality of identical unit-dosage forms packaged in a single container to be administered in segregated unit-dose form. Examples of multiple-dose forms include vials, blisters, or bottles.

In accordance with the present invention, Compound (A) may be present in the pharmaceutical composition (e.g. for oral administration) in an amount of about 0.5 mg to about 600 mg. In one aspect, the present invention relates to a pharmaceutical composition for oral administration wherein the final dosage form comprises Compound (A), in an amount of about 0.5 mg to about 600 mg, or about 5 mg to about 400 mg, or about 10 mg to about 150 mg. Preferably, the amount of Compound (A) in the final dosage form is about 0.5 mg, or about 5 mg, or about 10 mg, or about 15 mg, or about 20 mg, or about 25 mg, or about 30 mg, or about 35 mg, or about 40 mg, or about 45 mg, or about 50 mg, or about 60 mg, or about 70 mg, or about 80 mg, or about 90 mg, or about 100 mg, or about 120 mg, or about 140 mg, or about 150 mg, or about 180 mg, or about 200 mg, or about 220 mg, or about 240 mg, or about 250 mg, or about 270 mg, or about 300 mg, or about 320 mg, or about 350 mg, or about 370 mg, or about 400 mg, or about 430 mg, or about 450 mg, or about 480 mg, or about 500 mg, or about 550 mg, or of about 600 mg. More preferably, the amount is about 10 mg, or about 15 mg, or about 20 mg, or about 25 mg, or about 50 mg, or about 100 mg, or about 150 mg, or about 200 mg, or about 250 mg, or about 300 mg, or about 350 mg, or about 400 mg, or about 450 mg, or about 500 mg, or of about 600 mg. Preferably, the amount of Compound (A) in the final dosage form is about 10 mg, about 25 mg, about 35 mg, about 50 mg, about 75 mg or about 100 mg. More preferably, the amount of Compound (A) in the final dosage form is about 10 mg, about 25 mg, about 50 mg or about 100 mg.

In accordance with the present invention, the final dosage form comprises Compound (A), in an amount of about 10 mg. In another aspect of the present invention, the final dosage form comprises Compound (A), in an amount of about 20 mg. In another aspect of the present invention, the final dosage form comprises Compound (A), in an amount of about 25 mg. In another aspect of the present invention, the final dosage form comprises Compound (A), in an amount of about 35 mg. In another aspect of the present invention, the final dosage form comprises Compound (A), in an amount of about 50 mg. In yet another aspect of the present invention, the final dosage form comprises Compound (A), in an amount of about 100 mg.

A further aspect of the invention relates to a pharmaceutical composition (e.g. for oral administration), as defined herein, comprising at least one further active pharmaceutical ingredient.

Another aspect the invention provides a capsule for oral administration comprising an amount of about 0.5 mg to about 600 mg of a BTK inhibitor, such as Compound (A), at least one binder, optionally a surfactant, and at least one pharmaceutically acceptable excipient.

Another aspect of the invention provides a tablet, preferably a film-coated tablet, for oral administration comprising an amount of about 0.5 mg to about 600 mg of Compound (A), at least one binder, optionally a surfactant, and at least one pharmaceutically acceptable excipient.

The pharmaceutical composition (e.g. for oral administration), as disclosed herein, is useful, for example, as a medicine. In particular, the pharmaceutical composition (e.g. for oral administration) is useful as a medicine for the treatment or prevention of a disease or disorder mediated by BTK or ameliorated by inhibition of BTK, such as, for example, autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis, myasthenia gravis, Sjögren's syndrome, autoimmune hemolytic anemia, anti-neutrophil cytoplasmic antibodies (ANCA)-associated vasculitides, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including, but not limited to, multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease. Specifically, the present disclosure provides the use of said pharmaceutical composition in the treatment or prevention of a disease or disorder mediated by BTK or ameliorated by the inhibition of BTK selected from rheumatoid arthritis; chronic urticaria (preferably chronic spontaneous urticaria); Sjögren's syndrome, multiple sclerosis or asthma.

Another aspect of the invention also provides for the use of the pharmaceutical composition (e.g. for oral administration) as disclosed herein, for the manufacture of a medicament for a disease or disorder mediated by BTK or ameliorated by the inhibition of BTK, wherein the disease or disorder is selected from autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis, myasthenia gravis, Sjögren's syndrome, autoimmune hemolytic anemia, anti-neutrophil cytoplasmic antibodies (ANCA)-associated vasculitides, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including but not limited to multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease. Specifically, the present disclosure provides the use of the pharmaceutical composition (e.g. for oral administration) as disclosed herein, for the manufacture of a medicament for the disease or disorder mediated by BTK or ameliorated by the inhibition of BTK, wherein the disease or disorder is selected from rheumatoid arthritis; chronic urticaria (preferably chronic spontaneous urticaria); Sjögren's syndrome, multiple sclerosis or asthma.

Another aspect of the invention also provides a method of treating or preventing a disease or disorder mediated by BTK or ameliorated by the inhibition of BTK, comprising administering to a subject in need of such treatment or prevention, a pharmaceutical composition or a final dosage form as disclosed herein.

### Definitions

The term “pharmaceutically acceptable salts” refers to salts that can be formed, for example, as acid addition salts, preferably with organic or inorganic acids. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. The term “pharmaceutically acceptable” refers to those compounds, materials, compositions, and/or dosage forms which are suitable for use in contact with the tissues of human beings and animals without excessive toxicity, irritation, allergic response, other problem or complication, commensurate with a reasonable benefit/risk ratio.

The term “treat”, treating” or “treatment” of any disease or disorder refers to ameliorating the disease or disorder (e.g. slowing, arresting or reducing the development of the disease, or at least one of the clinical symptoms thereof). In addition those terms refer to alleviating or ameliorating at least one physical parameter including those which may not be discernible by the patient and also to modulating the disease or disorder, either physically (e.g. stabilization of a discernible symptom), physiologically (e.g. stabilization of a physical parameter), or both.

The term “prevent”, “preventing” or “prevention” of any disease or disorder refers to delaying the onset, or development, or progression of the disease or disorder.

The term “about”, as used herein, is intended to provide flexibility to a numerical range endpoint, providing that a given value may be “a little above” or “a little below” the endpoint accounting for variations one might see in the measurements taken among different instruments, samples, and sample preparations. The term usually means within 10%, preferably within 5%, and more preferably within 1% of a given value or range.

The terms “pharmaceutical composition” or “formulation” can be used herein interchangeably, and relate to a physical mixture containing a therapeutic compound to be administered to a mammal, e.g. a human, in order to prevent, treat, or control a particular disease or condition affecting a mammal. The terms also encompass, for example, an intimate physical mixture formed at high temperature and pressure.

The term “oral administration” represents any method of administration in which a therapeutic compound can be administered through the oral route by swallowing, chewing, or sucking an oral dosage form. Such oral dosage forms are traditionally intended to substantially release and/or deliver the active agent in the gastrointestinal tract beyond the mouth and/or buccal cavity.

The term “a therapeutically effective amount” of a compound, as used herein, refers to an amount that will elicit the biological or medical response of a subject, for example, ameliorate symptoms, alleviate conditions, slow or delay disease progression, etc. The term “a therapeutically effective amount” also refers to an amount of the compound that, when administered to a subject, is effective to at least partially alleviate and/or ameliorate a condition, a disorder, or a disease. The term “effective amount” means the amount of the subject compound that will engender a biological or medical response in a cell, tissue, organs, system, animal or human that is being sought by the researcher, medical doctor or other clinician.

The term “comprising” is used herein in its open ended and non-limiting sense unless otherwise noted. In a more limited embodiment “comprising” can be replaced by “consisting of”, which is no longer open-ended. In a most limited version it can include only feature steps, or values as listed in the respective embodiment.

The term “inert substrate”, as used herein, refers to a substance or a material that does not react with neither a chemically or biologically reactive substance, and will not decompose. For example, the inert substrate refers to a substance or a material which does not react chemically with the suspension (i.e. does not react chemically with the (b) mixture comprising compound (A) and at least one binder).

The term “glidant” or “gliding agent” as used herein, refers to a substance or a material that improves the flowability properties of the final blend.

The term “Disintegrant” or “disintegrating agent” as used herein, refers to a substance or a material added to oral solid dosage forms, e.g. tablet, to aid in their disaggregation, by causing a rapid break-up of solids dosage forms when they come into contact with moisture.

The term “binder” or “binding agent” is used herein interchangeably and is in its established meaning in the field of pharmaceutics. It refers to a non-active substance that is added alongside the active pharmaceutical ingredient (herein referred to as Compound (A)), e.g. adhesion to the inert substrate particles in case of compound (A) deposition or in case of tableting as a promoter of cohesive compacts which enables to form granules and which ensures that granules can be formed with the required mechanical strength. All binders, referred herein, are used in qualities suitable for pharmaceutical use and are commercially available under various brand names as indicated in the following examples:
Polyvinylpyrrolidone-vinyl acetate copolymer is commercially available under the trade name Copovidone (approximate molecular weight of 45000-70000). Copovidone (Ph. Eur.) is a copolymer of 1-ethenylpyrrolidin-2-one and ethenyl acetate in the mass proportion 3:2. It contains 7.0 to 8.0% of nitrogen and 35.3 to 42.0% of ethenyl acetate (dried substance). It can be commercialized under the name Kollidon® VA 64.
Polyvinyl pyrrolidone (INN Ph. Eur.) is commercially available under the trade name Povidone K30 or PVP K30 (approximate molecular weight 50000).
Carboxymethylcellulose (USP/NF) is also known as the calcium salt of a polycarboxymethyl ether of cellulose. It is commercially available under the trade name Carmellose Calcium.
Shellac (INN Ph. Eur.) is a commercially available resin excreted by the females of the insects Laccifer lacca Kerr, Kerria lacca Kerr, Tachardia lacca, Coccus lacca and Carteria lacca on various trees. Shellac composition is as follows: 46% Aleuritic acid (HOCH₂(CH₂)₅CHOHCHOH(CH₂)₇COOH), 27% Shellolic acid (a cyclic dihydroxy dicarboxylic acid and its homologues), 5% Kerrolic acid (CH₃(CH₂)₁₀(CHOH)₄COOH), 1% Butolic acid (C₁₄H₂₈(OH)(COOH)), 2% Esters of wax alcohols and acids, 7% Non-identified neutral substances (e.g. coloring substances, etc), and 12% Non-identified polybasic esters.
Polyvinyl alcohol (INN Ph. Eur.) is commercially available under the trade name Polyviol or PVA (approximate molecular weight 28000 to 40000).
Polyethylene glycol (Ph. Eur.) is commercially available under the trade name PEG-n, where “n” is the number of ethylene oxide units (EO-units) (approximate molecular weight up to 20000).
Polyvinyl alcohol-polyethylene glycol co-polymer also known as polyvinyl alcohol-PEG copolymer or PEG-PVA.
Polyethylene-propylene glycol copolymer, also known as α-Hydro-ω-hydroxypoly(oxyethylene)poly(oxypropylene) poly(oxyethylene) block copolymer (CAS 9003-11-6), is commercially available under the name poloxamer (INN Ph. Eur.). The poloxamer polyols are a series of closely related block copolymers of ethylene oxide and propylene oxide conforming to the general formula HO(C₂H₄O)ₐ (C3H6O)_{b}(C2H4O)ₐH.

The term “surfactant” or “surface active agent” refers to an organic compound that are amphiphilic, meaning they possess both a hydrophobic hydrocarbon chain (tail) and a hydrophilic head. Surfactants contain both a water-insoluble (or oil-soluble) component and a water-soluble component. Surfactants are classified as ionic (e.g. anionic or cationic) or non-ionic, according to their characteristic on dissociation.
Polysorbates is commercially available under the name Tween 80. It is also known in the literature under the names Polysorbate 80, PEO(20) sorbitan mono-oleate (INCI, former name Crillet 4 Super).
The term “nano-sized” or “nanoparticulate” refer to a particle with a particle size in range of about 100 nm to about 1000 nm

### Abbreviations

% w/w Percent weight by weight
° C. Degree Celsius
API Active pharmaceutical ingredient
AUC Area Under the Curve
AUCinf AUC curve up to infinite time
AUClast AUC up to the last measurable concentration
Cmax Maximum concentration
CV % Coefficient of variation (%)
DR Dissolution rate
DSC Differential scanning Calorimetry
g/min Gram per minute
HPLC High-Performance Liquid Chromatography
HR-XRPD High resolution-X-ray Powder Diffraction
INCI International Nomenclature of Cosmetic Ingredients
INN International nonproprietary name
Kg/g/mg/ng/μg Kilogram/Gram/Milligram/Nanogram/Microgram
kN Kilo Newton
LCMS Liquid Chromatography-Mass Spectrometry
mL/L Milliliters/Liters
MRT Mean resistance time
nm/μm Nanometer/Micrometer
PCS Photon correlation spectroscopy
Ph. Eur. European Pharmacopoeia (9^{th} edition)
PK Pharmacokinetic
RH Relative humidity
Rpm Rotation per minutes
RRT Relative retention time
RT Room temperature
SD and RSD Standard deviation and relative standard deviation
SEM Scanning Electron Microscopy
SLS Sodium Lauryl sulfate
TFA Trifluoroacetic acid
TGA Thermogravimetric analysis
Tmax Time to peak maximum concentration (Cmax)
US Ultrasonic sonication
USP United States Pharmacoepia
USP/NF United States Pharmacoepia/National Formulary
w/v weight by volume
w/w weight by weight
XRPD X-ray Powder Diffraction

### EXAMPLES

The following examples illustrate the invention and provide support for the disclosure of the present invention without limiting the scope of the invention.
Analytical Centrifugation (AC), e.g., LUMiSizer, LUM GmbH Germany, SEPView 6.1.2570.2022. Wet dispersion method using purified water solution for dilution of suspension to appropriate attenuation level with about 10 to 70% transmission of first measurement profile. The reported results for X10, X50, X90 are intensity weighted.
Photon Correlation Spectroscopy (PCS), e.g., Zetasizer Nano ZS, Malver Panalytical Ltd., UK, Version 7.3. Wet dispersion method using 0.1 mM NaCl solution (in purified water) for dilution of suspension to appropriate attenuation level with about 2 to 9 attenuator index. The reported results for Xmean are intensity weighted. In particular, the attenuator index is 5. Preferably, the measurement is carried at 25° C. Further preferred settings of measurement systems are as follow:
Cell: disposable sizing cuvette
Count Rate (KcPs): 315
Duration: 60 s
Measurement position (mm): 4.65
Zeta-potential, e.g., Zetasizer Nano, Malvern Panalytical Ltd., UK
Scanning electron micrographs (SEM), e.g., Supra 40, Carl Zeiss SMT AG, Germany
Dynamic viscosity, e.g., Haake Mars, ThermoFisher Scientific GmbH, Germany
Sinker method, e.g., Balance with sinker for liquid density, Mettler Toledo GmbH, Switzerland
Microbial enumeration test (MET).

### Example 1: Preparation of the Granule Particles

The role of the inert substrate was evaluated by preparing adding different (b) mixtures, as defined herein, on different type of (a) inert substrate, e.g. mannitol and lactose.

Different granule particle compositions were prepared by suspending the binder polyvinylpyrrolidone-vinyl acetate copolymer (copovidone), Compound (A), as defined herein and the surfactant sodium lauryl sulfate in a liquid medium such as purified water. The different variants are described in Table 1.

TABLE 1
Study of different granule particle variants and particle size distribution

| Variants | P1 | P2 | P3 | P4 | P5 | P6 | P7 | P8 | P9 |
|---|---|---|---|---|---|---|---|---|---|
| Compound (A) | 10.3 | 20.1 | 20.1 | 10.0 | 20.0 | 20.0 | 20.0 | 1.95 | 1.61 |
| polyvinylpyrrolidone- | 30.9 | 4.0 | 4.0 | 30.0 | 20.0 | 10.0 | 20.0 | 2.99 | 0.32 |
| vinyl acetate | | | | | | | | | |
| copolymer | | | | | | | | | |
| Sodium lauryl | 1.7 | 0.2 | 0.2 | 0.1 | 0.2 | 0.2 | 1.0 | 0.15 | 0.02 |
| sulfate(SLS) | | | | | | | | | |
| Mannitol SD200 | 57.1 | | 75.7 | 59.9 | 59.8 | 69.8 | 59.0 | | 98.05 |
| Lactose spray dried | | 75.7 | | | | | | 94.91 | |
| Total | 100.0 | 100.0 | 100.0 | 100.0 | 100.0 | 100.0 | 100.0 | 100.00 | 100.0 |
| Drug substance/ | 1:3 | 5:1 | 5:1 | 1:3 | 1:1 | 2:1 | 1:1 | 1:1.5 | 5:1 |
| polymer ratio | | | | | | | | | |
| Particle size [nm] | 131 | 308 | 412 | 156 | 151 | 236 | 144 | 130 | 491 |
| (water/no US) - | | | | | | | | | |
| single value | | | | | | | | | |
| particle size [nm] | 127 | 219 | 264 | 145 | 142 | 166 | 140 | 123 | 247 |
| (water/1 min US + | | | | | | | | | |
| diluted 1:10) - | | | | | | | | | |
| MEAN from 2 | | | | | | | | | |
| values | | | | | | | | | |
| Particle size of | 114 | 114 | 114 | 128 | 128 | 128 | 128 | 114 | 114 |
| starting suspension | | | | | | | | | |
| [nm] | | | | | | | | | |

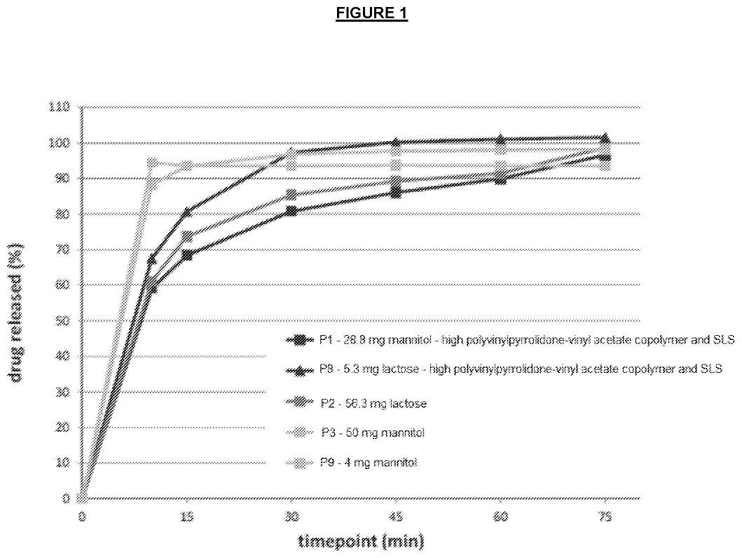

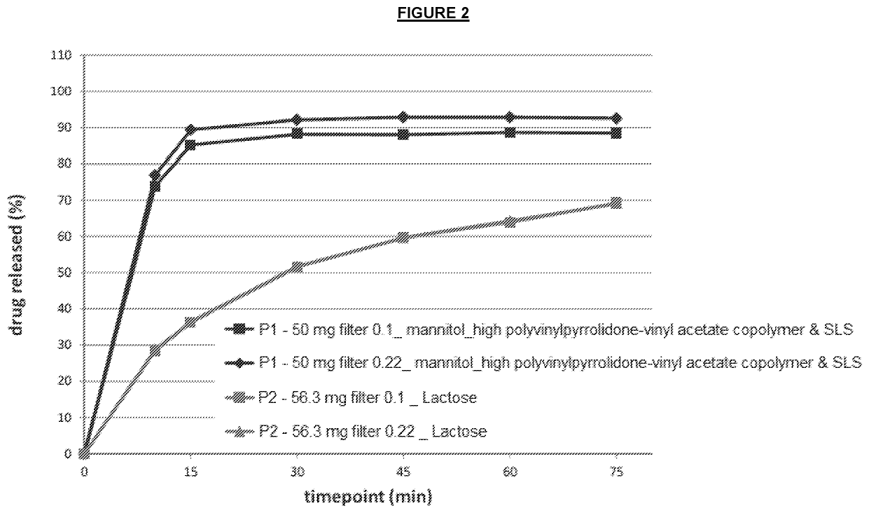

It was observed that variants P1, P4, P5, and P7 containing higher ratio of polyvinylpyrrolidone-vinyl acetate copolymer (copovidone) showed best re-suspendability compared to the starting suspension. Variants P1 and P7 containing higher ratio of SLS are also superior in re-suspendability. Variant P7 was selected as the optimized granule composition in terms of copovidone and SLS ratio, thus the amount to be sprayed on the inert substrate (carrier particle) allow a drug load of 20%, based on the total weight of the granule particle, in a reasonable processing time. The dissolution performance of the different variant of granule particle compositions was evaluated to ensure the dissolution profile was in a good range. The dissolution rate is measured by conventional method (paddle method according to Pharm. Eur. 2.9.3 “Dissolution Test for Solid Dosage Forms” or US Pharmacopeia <711> “Dissolution” or Japanese Pharmacopiea <6.10> “Dissolution Test), as it can be seen in FIG. 1 and FIG. 2, by adding the granule particles prepared as mentioned in Table 1 into capsules (e.g. hard gelatin capsule). FIG. 1 shows the dissolution rate at pH 2 and provides sink conditions (solubility of 0.3 mg/mL) for the tested doses of 50 mg independently from the particle size of the drug substance. Some of the tested capsules comprising the above mentioned granule particles, the disintegration and dispersion of the content was delayed, leading to a delayed dissolution rate (DR) profile at paddle 50 rpm. In order to improve disintegration and dispersion of the formulation content, specifically at pH 2, the addition of at least one pharmaceutically acceptable excipient (e.g. as an external phase) was investigated. FIG. 2 shows that the maximum solubility of Compound (A) at pH 3 is reached at 90% for the 50 mg dose in 900 mL. P1 granule particles, as seen in FIG. 2, with a high level of polyvinylpyrrolidone-vinyl acetate copolymer and SLS, showed a good re-suspendability, while P2 granule particles, with low level of polyvinylpyrrolidone-vinyl acetate copolymer and SLS, did not achieve good levels of re-suspendability. No significant difference of separation behavior between 0.1 and 0.22 μm filters were observed. The dissolution profile of P2 granule particles for both filters is completely overlapping (as depicted in FIG. 2).

The granule particles P1, P2, P3, P7 (prepared according to Table 1), and one granule particle with additional excipients (P7″) were then evaluated in male beagle dogs, as summarized in Table 2 and Table 3.

TABLE 2
Dog PK study - formulations P1, P2, P3, P7 and P7

| | Variants | | | | | | | | | |
|---|---|---|---|---|---|---|---|---|---|---|
| | P1 | | P2 | | P3 | | P7 | | P7″ | |
| | [%] | [mg] | [%] | [mg] | [%] | [mg] | [%] | [mg] | [%] | [mg] |
| Compound (A) | 10.3 | 30.0 | 20.1 | 30.0 | 20.1 | 30.0 | 20.0 | 30.0 | 17.3 | 30.0 |
| polyvinylpyrrolidone-vinyl | 30.9 | 90.0 | 4.0 | 6.0 | 4.0 | 6.0 | 20.0 | 30.0 | 17.3 | 30.0 |
| acetate copolymer | | | | | | | | | | |
| Sodium lauryl sulfate(SLS) | 1.7 | 5.0 | 0.2 | 0.3 | 0.2 | 0.3 | 1.0 | 1.5 | 0.9 | 1.5 |
| Mannitol SD200 | 57.1 | 166.7 | | | 75.7 | 112.8 | 59.0 | 88.5 | 51.1 | 88.5 |
| Lactose spray dried | | | 75.7 | 112.8 | | | | | | |
| Magnesium stearate | | | | | | | | | | 0.9 |
| Sodium bicarbonate | | | | | | | | | | 5.2 |
| Microcrystalline cellulose | | | | | | | | | | 17.3 |
| Total | 100.0 | 291.7 | 100.0 | 149.1 | 100.0 | 149.1 | 100.0 | 150.0 | 100.0 | 173.4 |

TABLE 3
Dog PK results

| Variants | P1 | | P2 | | P3 | | P7 | | P7″ | |
|---|---|---|---|---|---|---|---|---|---|---|
| Cmax (ng/mL) | 427 ± 174 | | 69.4 ± 56.1 | | 105 ± 64.1 | | 398 ± 215 | | 332 ± 299 | |
| Cmax/dose | 141 ± 58 | | 23.9 ± 20.6 | | 34.7 ± 21.5 | | 134 ± 76.3 | | 110 ± 102 | |
| (ng/mL)/(mg/kg) | | | | | | | | | | |
| Tmax (h) | 0.5 | [0.5-0.5]* | 1.5 | [0.5-2]* | 1.5 | [1-2]* | 0.75 | [0.5-1]* | 2 | [0.5-2]* |
| Tlast (h) | 24 | [7-24]* | 24 | [7-24]* | 24 | [7-24]* | 7 | [7-7]* | 7 | [7-24]* |

| | | | | | |
|---|---|---|---|---|---|
| AUClast (h · ng/mL) | 741 ± 413 | 266 ± 132 | 303 ± 159 | 719 ± 366 | 531 ± 370 |
| AUClast/dose | 246 ± 141 | 89.1 ± 44.2 | 100 ± 53.1 | 241 ± 129 | 173 ± 126 |
| (h · ng/mL)/(mg/kg) | | | | | |
| AUCinf Obs | 767 ± 422 | 403 ± 232 | 372 ± 209 | 727 ± 367 | 716 ± 327 |
| (h · ng/mL) | | | | | |
| AUCinf/dose | 26.9 ± 14.8 | 13.9 ± 8.01 | 13 ± 7.29 | 26.7 ± 13.5 | 26.1 ± 12 |
| ((h · ng/mL)/(mg/kg)) | | | | | |

Mean ± SD are presented,
*median [time range]

The results showed that the inter-subject variations of Cmax and AUClast evaluated by CV (%) were 40.7-90.1% and 49.6-69.7%, respectively among formulations. The maximum concentrations (Cmax) were reached between 0.5-2 hours (median) among formulations. Based on the outcome of this study it was concluded, that re-suspendability is supporting higher in-vivo exposure in dogs and can be used as a selection criteria for rating between different variants (e.g. P1, P2, P3, P7 and P7″).

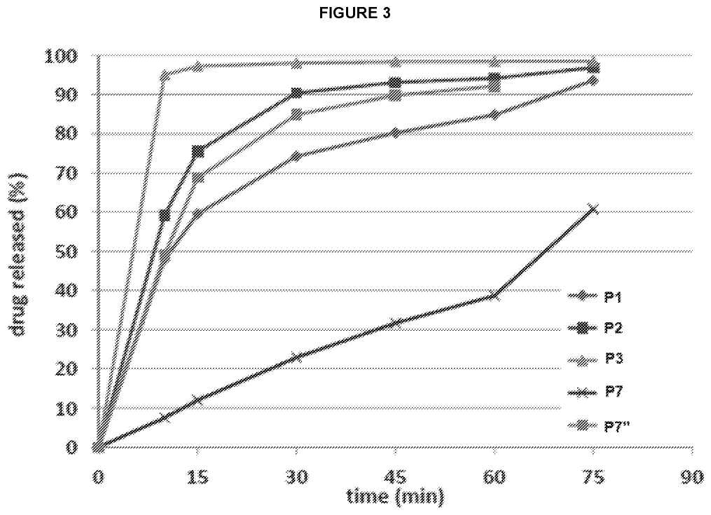

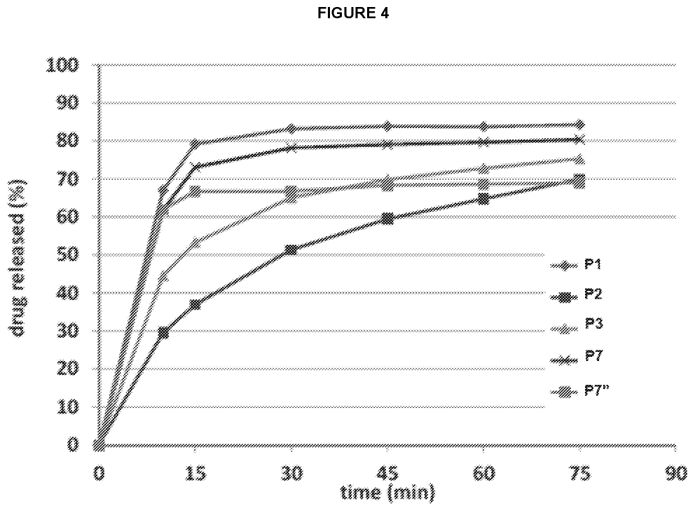

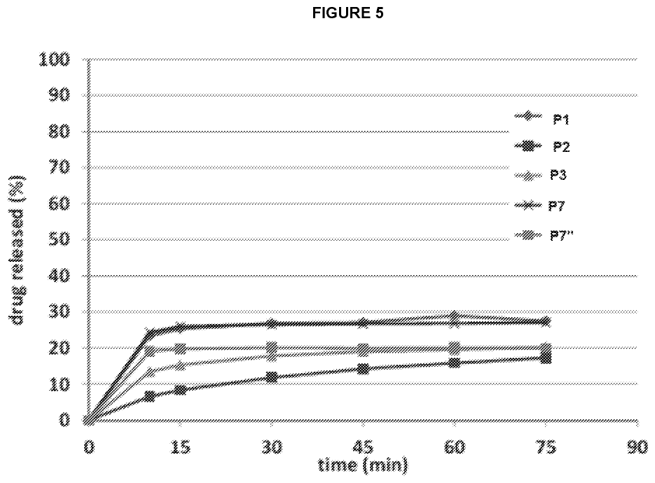

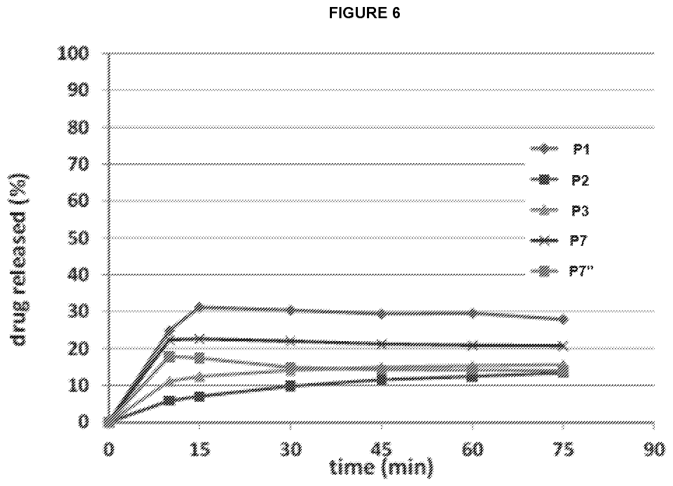

In order to have a better understanding of the formulation/pH profile, the dissolution rate profiles of the formulations described in Table 2 and Table 3 were measured at pH 2, pH 3, pH 4.5 and pH 6.8. The results have been summarized in FIG. 3, FIG. 4, FIG. 5, and FIG. 6. It was shown that the formulations behaviors were changing between pH 2 and pH 3. Formulations containing low amount of binder (polyvinylpyrrolidone-vinyl acetate copolymer) and surfactant (sodium lauryl sulfate) were faster in dissolution rate compared to the formulations containing higher amounts. It was observed that the slow dissolution rate for the formulations is related to an observed gelling behavior, which is not seen at low amounts of binder and surfactant. At pH 3 and higher pH no formulation shows a gelling effect and content of all capsules is dispersing within the first 10 min.

### Example 2: Role of the Particle Sizes

The role of the particle size of Compound (A), as defined herein, was also investigated to have a better understanding of the particle size distribution, of the dissolution profile of the different formulations, and also to understand the impact of the particle size on the formulation. The granule particles were prepared according to Example 1 procedure.

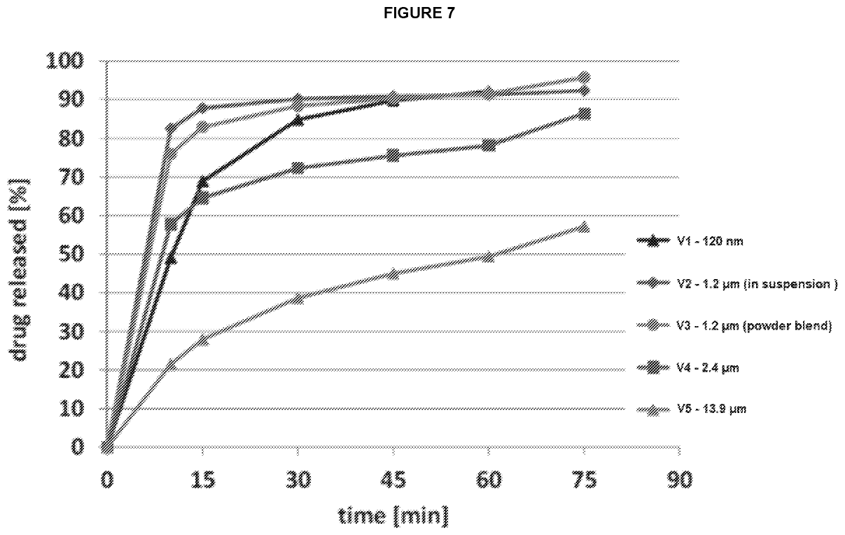

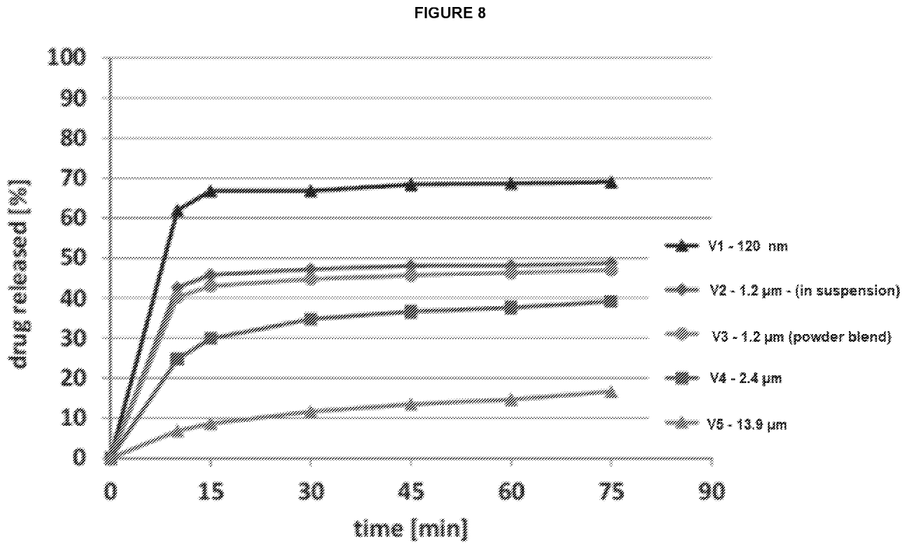

Several particle sizes for the drug substance (i.e. Compound (A)) were investigated as mentioned below (with a 50 mg dose of Compound (A)) and the results are depicted in FIG. 7 and FIG. 8:
V1=particle size of 120 nm—nanoparticulate formulation (wet milled suspension).
V2=particle size of 1.2 μm—as a non-wet milled suspension.
V3=particle size of 1.2 μm—as a powder blend.
V4=particle size of 2.4 μm—as a powder blend.
V5=particle size of 13.9 μm—as a powder blend.

Already at pH 2, a strong particle size impact was noticed on the dissolution rate profile as observed on FIG. 7. Formulation V5 (13.9 μm) shows a big gap at infinity of about 40% from full release and a delayed profile. Particle size 2.4 μm (V4) compared to 1.2 μm (V2 and V3) shows a clear drop in dissolution rate and also a delayed profile. Comparison between the V1 formulation with a particle size of 120 nm and the formulations with a particle size of 1.2 μm (V2 and V3) showed that the formulations with a particle size of 1.2 μm are faster at the beginning of the profile but ultimately are reaching the same endpoints (FIG. 7). The particle size impact seen in the dissolution rate (DR) profiles at pH 2 is even more obvious at pH 3 as depicted in FIG. 8. As showed in FIG. 8, the gap between the 13.9 μm particle size (V5) and the 120 nm particle size (V1) is about 60%. The fastest micron-sized drug substance (V2) shows a gap of about 20% compared to V1.

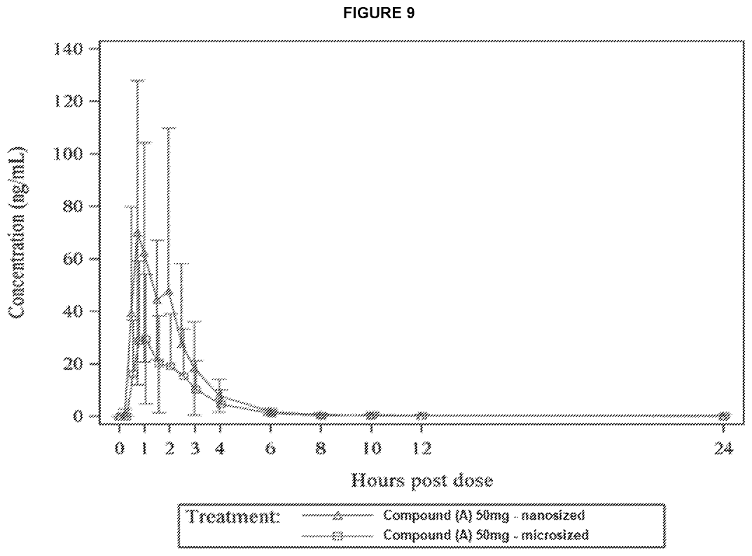

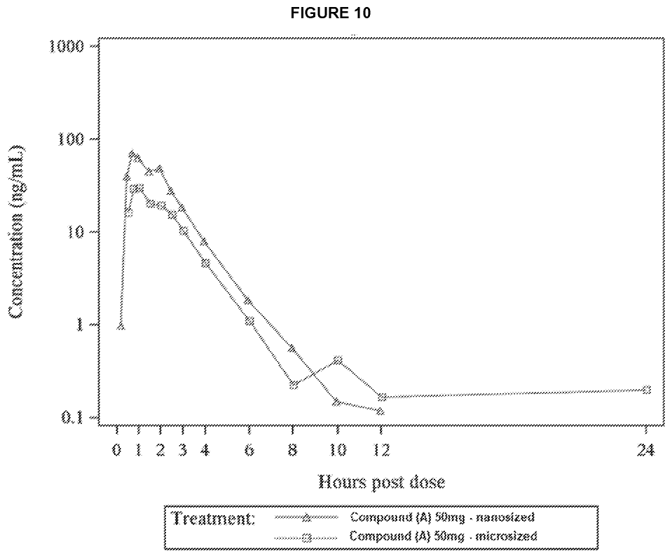

The role of the particle size (e.g. micron-sized or nano-sized) and the effects of the formulation on the PK were evaluated in 13 male beagle dogs, after administration of a 50 mg dose of Compound (A). The arithmetic mean (SD) blood concentration-time plot per treatment is displayed in FIG. 9 and FIG. 10. The PK parameters are summarized in Table 4 below.

TABLE 4
Summary statistics of PK parameter values -
effect of the particle size

| PK parameter | Nano-sized | Micron-sized |
|---|---|---|
| (unit) | N = 12 | N = 13 |
| Cmax (ng/mL) | 95.6 ± 66.3 (69.3) | 40.6 ± 31.1 (76.6) |
| | 94.4 (9.54-239) [12] | 34.1 (4.19-96.2) [13] |
| Tmax (h) | 0.750 (0.733-2.00) [12] | 1.00 (0.733-2.50) [13] |
| AUClast (h*ng/mL) | 136 ± 80.2 (59.0) | 66.2 ± 51.1 (77.2) |
| | 127 (15.7-289) [12] | 62.3 (4.61-131) [13] |
| AUCinf (h*ng/mL) | 140 ± 79.9 (57.3) | 90.7 ± 39.3 (43.4) |
| | 135 (18.2-290) [12] | 101 (30.3-131) [6] |
| T½ (h) | 0.962 (0.867-7.36) [12] | 0.824 (0.649-8.62) [6] |
| MRT (h) | 1.89 ± 0.339 (17.9) | 2.20 ± 1.21 (54.8) |
| | 1.80 (1.48-2.64) [12] | 2.17 (0.839-4.85) [13] |

Statistics are Mean ± SD (CV %)
Median (Min-Max)[n]
CV % = Coefficient of variation (%) = SD/Mean × 100
For Tmax and T½, only Median (Min-Max)[n] are presented

A slightly earlier median Tmax was observed when the formulation comprising nano-sized particles of Compound (A) was given (0.75 hour) compared to the micron-sized formulation (1.0 hour). The geo-mean CV % for Cmax was 117.3% for the nano-sized formulation whereas it was 178.1% for micron-sized formulation. Similarly, the geo-mean CV % for AUClast was 94.7% for the nano-sized formulation and 212.5% for the micron-sized formulation. The statistical analysis of the effect of the particle size on the PK showed that the micron-sized formulation achieved only 40.5% of the AUC (geo-mean ratio: 0.405 with 90% Confidence interval (CI): 0.215, 0.763) and 40.9% of Cmax (geo-mean ratio: 0.409 with 90% CI: 0.233, 0.717) of the nano-sized formulation. In addition, considerable lower variability was seen when compared to the micron-sized formulation.

### Example 3: Composition of the Suspension

The formulation composition of a wet-media milled Compound (A) suspension was investigated with regard to increase of drug concentration in suspension considering polyvinylpyrrolidone-vinyl acetate copolymer (Copovidone) and sodium lauryl sulfate (SLS) as excipients. Several formulation compositions were evaluated as shown in Table 5 and Table 6.

TABLE 5
Formulation compositions comprising Compound (A)

| | | Polyvinylpyrrolidone- | | |
|---|---|---|---|---|
| | | vinyl acetate | Sodium | |
| | Compound | copolymer | lauryl | Water |
| | (A) | (Copovidone) | sulfate(SLS) | (purified) |
| Batch | /% w/w | /% w/w | /% w/w | /% w/w |
| F1 | 20 | 4 | 0.1 | 75.9 |
| F2 | 25 | 4 | 0.1 | 70.9 |
| F3 | 30 | 4 | 0.1 | 65.9 |
| F4 | 35 | 4 | 0.1 | 60.9 |

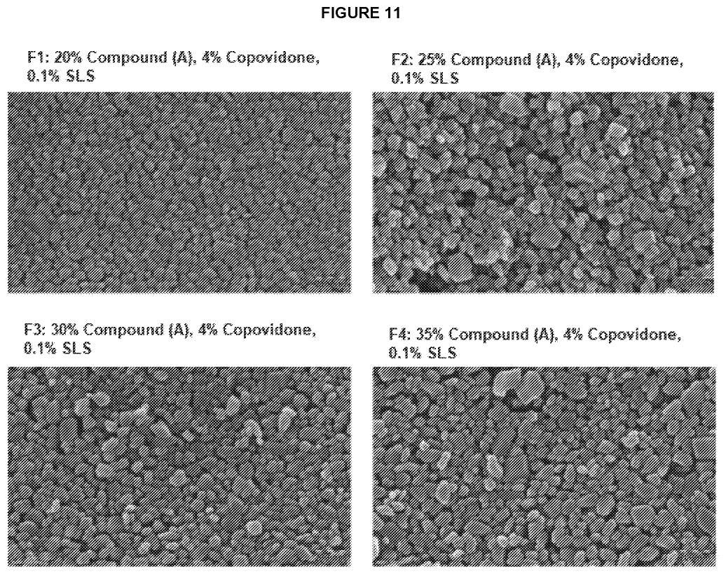

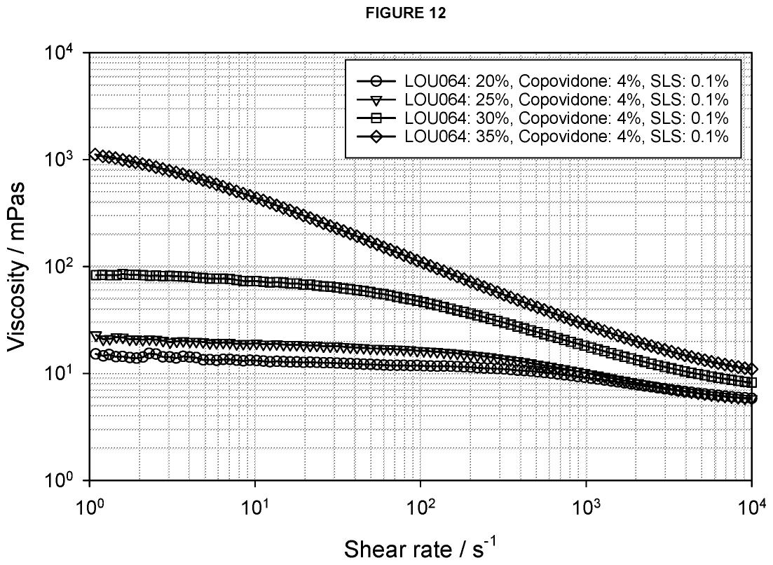

The obtained experimental results are summarized with regard to particle size by Analytical Centrifugation (AC), Photon Correlation Spectroscopy (PCS) and Zeta-potential in Table 6 below. Scanning electron micrographs of the drug particles obtained and dynamic viscosity identified by a rotational ramp up rheology test at 25° C. temperature are depicted in FIG. 11 and FIG. 12.

TABLE 6
Particle size and Zeta-potential of the
suspension comprising Compound (A)

| | | | | Particle size by PCS | |
|---|---|---|---|---|---|
| | | | | (without US/with 1 | Zeta- |
| | Particle size by AC | | | min US) | potential |
| Batch | x₁₀/nm | x₅₀/nm | x₉₀/nm | xₘₑₐₙ/nm | /mV |
| F1 | 74 | 106 | 162 | 98/99 | −31.7 |
| F2 | 79 | 110 | 158 | 110/108 | −26.8 |
| F3 | 79 | 109 | 153 | 114/111 | −28.3 |
| F4 | 86 | 117 | 158 | 136/126 | −25.8 |

The formulation composition of wet-media milled suspension comprising Compound (A) based on 25% w/w drug concentration was selected based on appropriate particle size and viscosity obtained for the screening experiments, for different compositions of excipients such as steric stabilizer, respectively binder (e.g. polyvinylpyrrolidone-vinyl acetate copolymer) and surfactant (e.g. sodium lauryl sulfate). The experiments were performed at standardized equipment and process parameter settings for adequate comparison. The investigated formulation compositions are shown in Table 7 below.

TABLE 7
Formulation compositions for the wet media milled
suspension of Compound (A) optimization trials

| | | Polyvinylpyrrolidone- | | |
|---|---|---|---|---|
| | Compound | vinyl acetate copolymer | Sodium lauryl | Water |
| | (A) | (Copovidone) | sulfate (SLS) | pruified |
| Batch | /% w/w | /% w/w | /% w/w | /% w/w |
| F5 | 25 | 3 | 0.1 | 71.9 |
| F6 | 25 | 3.5 | 0.1 | 71.4 |
| F2 | 25 | 4 | 0.1 | 70.9 |
| F7 | 25 | 4.5 | 0.1 | 70.4 |
| F8 | 25 | 4 | 0.05 | 70.95 |
| F9 | 25 | 4 | 0.15 | 70.85 |

The obtained experimental results are summarized in Table 8 below with regard to particle size using Analytical Centrifugation (AC), Photon Correlation Spectroscopy (PCS) Zeta-potential and pH.

TABLE 8
Particle size (AC and PCS), Zeta-potential
and pH of the wet media milled suspension

| | | | | Particle size by | | |
|---|---|---|---|---|---|---|
| | | | | PCS (without | Zeta- | |
| | Particle size by AC | | | US/with 1′ US) | potential | |
| Batch | x₁₀/nm | x₅₀/nm | x₉₀/nm | xₘₑₐₙ/nm | /mV | pH |
| F5 | 75 | 106 | 150 | 112/105 | −30.4 | 5.4 |
| F6 | 77 | 108 | 163 | 111/107 | −28.2 | 5.5 |
| F2 | 77 | 112 | 176 | 108/107 | −29.8 | 5.5 |
| F7 | 73 | 104 | 163 | 111/106 | −30.9 | 5.6 |
| F8 | 96 | 115 | 142 | 213/174 | −28.8 | 5.3 |
| F9 | 94 | 284 | 585 | 113/115 | −39.4 | 5.7 |

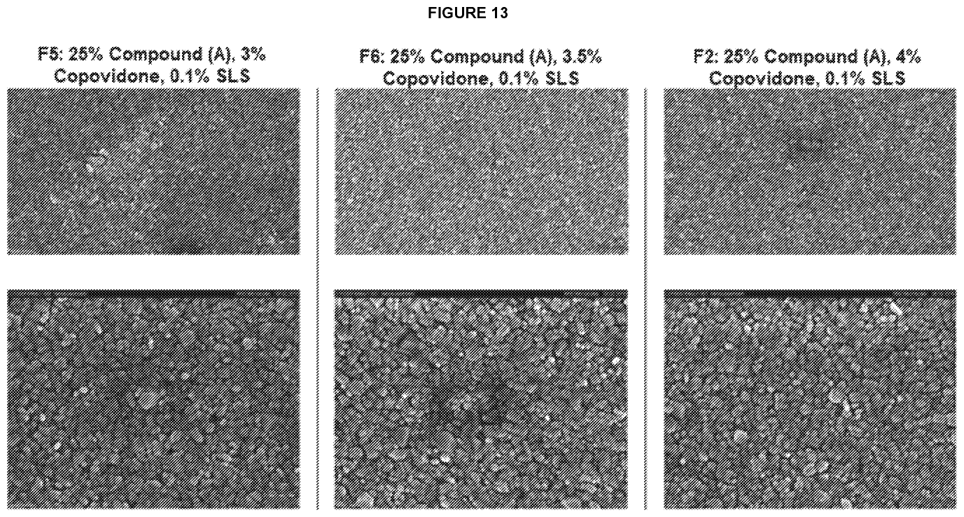

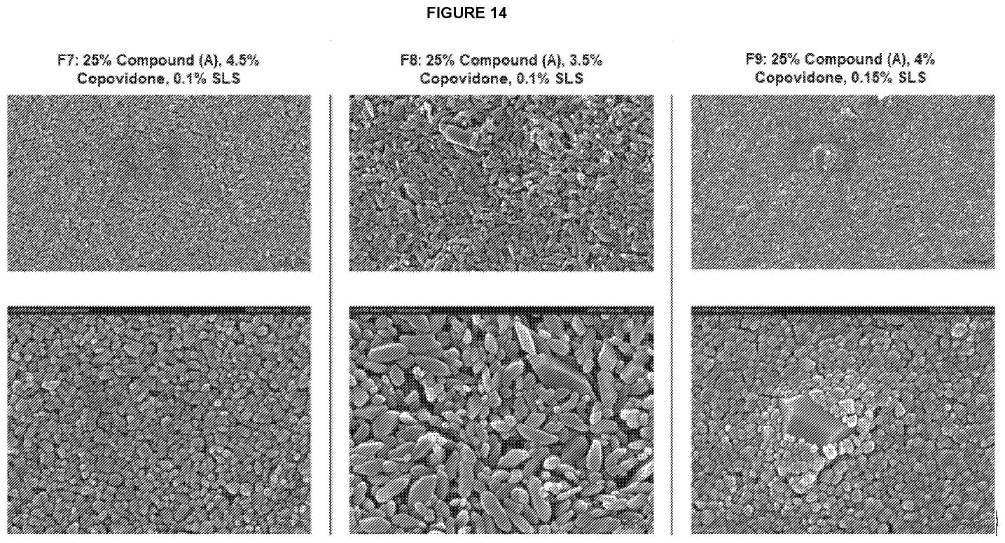

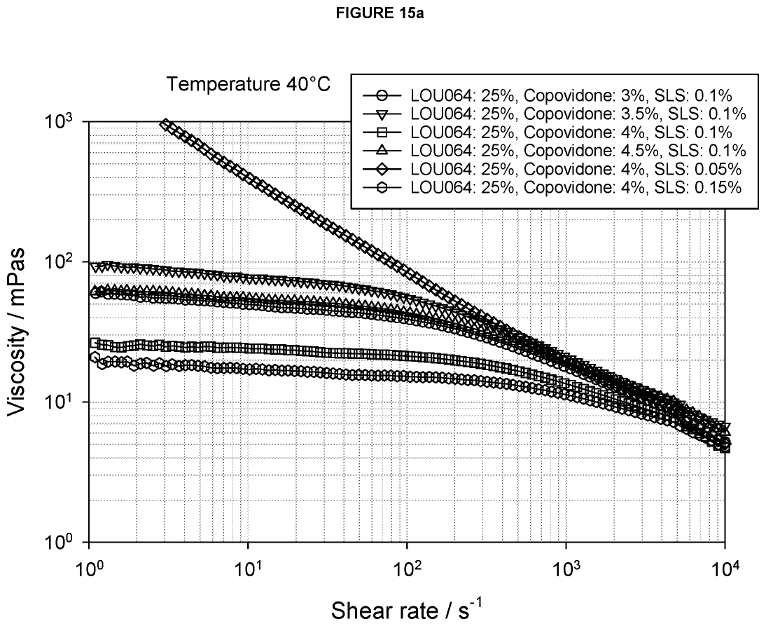

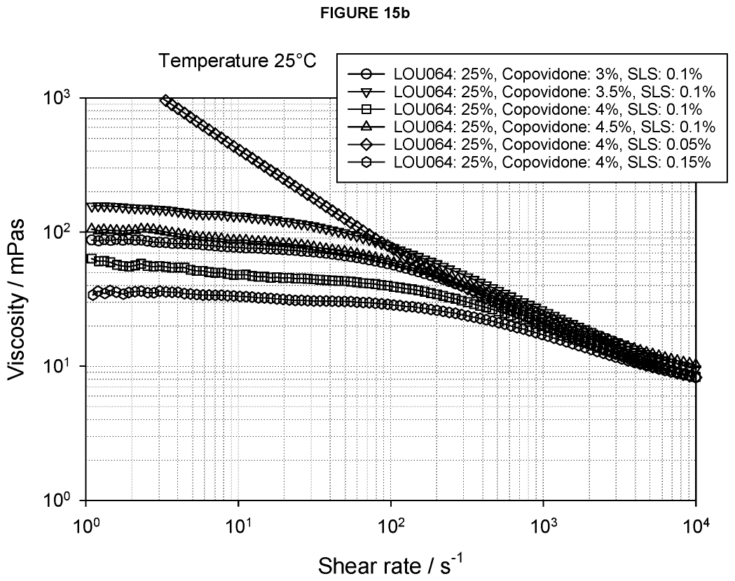

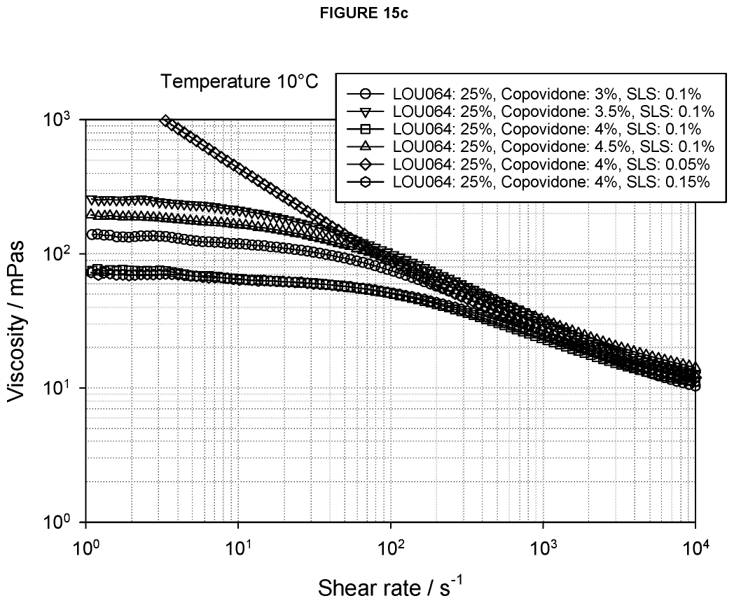

Scanning electron micrographs of the drug particles are shown in FIG. 13 and FIG. 14. Dynamic viscosity was characterized by a rotational ramp-up rheology test at 10° C., 25° C. and 40° C. as shown in FIG. 15. Assay and density of the wet-media milled suspension comprising Compound (A) was characterized by HPLC and by weight using sinker method, respectively. The results are summarized in the Table 9 below.

TABLE 9
Assay and density of the wet media milled suspension

| | | Density at |
|---|---|---|
| | Assay | 22° C. |
| Batch | /% of declared content in mg/mg | /g/cm³ |
| F6 | 97.6 | 1.081 |
| F2 | 92.6 | 1.083 |
| F7 | 97.1 | 1.072 |
| F8 | 88.5 | 0.995 |
| F9 | 93.4 | 1.072 |

Formulation composition of the wet media suspension F2 (25% w/w Compound (A), 4% w/w copovidone binder, and 0.1% w/w SLS surfactant) was selected based on appropriate particle size data, low dynamic viscosity across the shear rates tested by rotational rheology, low complex viscosity at rest, respectively low frequency, and no or low indication of particle agglomeration as identified by Photon Correlation Spectroscopy comparing particle sizes with and without ultrasound and, in addition, the linear behavior at low frequency as identified by the frequency sweep test. The other formulation compositions were not considered suitable for development, due to higher viscosities (F5, F6, F7, and F8). In addition, particle growth by Ostwald ripening was observed at elevated sodium lauryl sulfate (SLS) concentration (F9).

Composition F2 have low viscosity which is advantageous regarding (a) quality: homogeneity, and (b) operation: handling of suspension, downstreaming of suspension into dry product (granules) using spray processes.

### Milling Process:

Formulation composition Compound (A): 25% w/w, Copovidone: 4% w/w and SLS: 0.1% w/w was scaled up to a batch size of 6 liter using the following equipment and process parameters: Grinding chamber volume of 600 ml, grinding media made from Zirconia, grinding media diameter of 100 μm, grinding media fill level in grinding chamber of 80% v/v, stirrer tip speed of 9 m/s, suspension inlet temperature of about 19° C., suspension outlet temperature of about 23° C., suspension flow of 7 l/h during ramp up of process and increased up to 33 l/h after 1 hour processing, milling duration of 8 hours.

The particle size of compound (A) was measured by PCS and such process allowed reduction of particle size between about 110 nm to about 130 nm.

### Example 4: Capsule Formulation

After development of the suspension composition for spray granulation and testing of several inert substrate (carrier particles) for the spray granulation, the granules were filled into a capsule. It was observed that during the dissolution rate, the capsule disintegration and dispersion of the carrier particles do not perform well at pH 2 (as seen in Example 1, Example 2 and Example 3), it was not possible to fill directly the carrier particles in capsules without further formulation steps. Thus, the presence of an external phase was investigated by testing different pharmaceutically acceptable excipients (e.g. disintegrants, filler) to improve the poor capsule disintegration and dispersion at pH 2.

To evaluate the role of the excipients, the granule particles were prepared as mentioned in the above examples, at a dose of 10 mg, 20 mg and 50 mg, using micron-sized particle sizes of Compound (A). Then, the granule particles were mixed with at least one pharmaceutically acceptable excipient and were encapsulated in hard gelatin capsule of size 0.

TABLE 10
Capsule formulation
Amount per 1 capsule (mg/unit)

| | Compound | Compound | Compound |
|---|---|---|---|
| Ingredients | (A) | (A) | (A) |
| Amount per batch (g)¹ | 10 mg | 25 mg | 50 mg |
| Wet media milling | | | |
| Compound (A) | 10.00 | 25.00 | 50.00 |
| polyvinylpyrrolidone-vinyl | 2.00 | 5.00 | 10.00 |
| acetate copolymer | | | |
| Sodium Lauryl Sulfate (SLS) | 0.050 | 0.125 | 0.250 |
| Water purified² | (37.950) | (94.875) | (189.750) |
| Suspension for spray | | | |
| granulation | | | |
| polyvinylpyrrolidone-vinyl | 8.00 | 20.00 | 40.00 |
| acetate copolymer | | | |
| Sodium Lauryl Sulfate (SLS) | 0.45 | 1.125 | 2.250 |
| Water purified² | (66.5) | (60.00) | (120.00) |
| Total amount of | (125.00) | (206.125) | (412.250) |
| spray suspension | | | |
| Carrier | | | |
| Mannitol SD200 | 104.50 | 73.750 | 147.50 |
| Spray granuled inner phase | 125.00 | 125.00 | 250.00 |
| Outer Phase | | | |
| microcrystalline cellulose³ | 34.225 | 34.225 | 68.450 |
| Sodium Bicarbonate | 0.825 | 0.825 | 1.650 |
| Magnesium stearate | 4.950 | 4.950 | 9.900 |
| Final blend | 165.00 | 165.00 | 330.00 |
| Capsule shell | 96.00 | 96.00 | 96.00 |
| (theoretical weight) | | | |
| Total capsule weight | 261.00 | 261.00 | 426.00 |

¹Theoretical amounts of the described batch size
²The water is removed during spray granulation process.
³Compensation material for variation in granule content is microcrystalline cellulose (e.g. Avicel PH101 ®)

### Assay Stability Data of the Capsule

The suspension comprising compound (A), as defined herein, was put on technical stability. No significant change of appearance, particle size by PCS, microscopy and assay occurs up to 10 weeks storage at storage condition 40° C._75 relative humidity (RH), and up to 9 months storage at storage conditions 5° C./ambient RH and 25° C._60 RH. At relative retention time (RRT) of 0.81 a degradation product was observed to form in samples stored at 25° C._60% RH and 40° C._75% RH. It increased with increasing storage temperature and time (25° C._60% RH: up to 0.23% after 9 months, 40° C._75% RH: up to 0.34% after 10 weeks). To avoid this degradation product the suspension was stored in the fridge and stability results showed that the degradation product remains unchanged at <0.05% after storage in the fridge (5° C./ambient) for up to 9 months. Aside from the degradation product at RRT of 0.81, no other significant change or increase of impurities was observed at the different storage conditions and storage durations tested. After storage for 8 weeks at 5° C./ambient and 25° C._60% RH, no microbial contamination was detected by means of Microbial enumeration test (MET).

### Example 5: Study of the External Phase Composition

The impact of formulation factors on quality attributes of compound (A) 50 mg tablet core was explored. Study factors were filler ratio, disintegrant level and type, glidant level and lubricant level and type.

For this study, fluid bed granulator with top spray configuration was the selected technology for the development. For this study, one granule composition was selected containing 40% w/w of drug load, 20% w/w of copovidone and 0.2% w/w of sodium lauryl sulfate. A design of experiments (DOE) was carried out to evaluate and improve the blend and tablet cores properties at lab-scale (i.e. tablet batch size of 250 g). The experiment screened and assessed formulation flowability and compactability as a result of some variables (i.e. filler ratio, the disintegrant type, the amount of disintegrant, the amount of glidant, the lubricant type and the amount of lubricant).

The purpose of this study was primarily to assess the release of compound (A) with regards to different 50% w/w external phase compositions on the selected granule (see granule composition in Table 11). The study focuses only on granulation and tableting process steps.

TABLE 11
Selected granule composition studied in the experiment

| Material | (%)w/w of granule |
|---|---|
| Compound (A) (free base) | 40.00 |
| Copovidone [Kollidon VA64] | 20.00 |
| Sodium Lauryl Sulfate [Duponol C] | 0.20 |
| Mannitol SD200 | 39.80 |
| Total | 100.00 |

The design used was a screening design of 6 factors (Table 12) in 12 designs run (table 13)

TABLE 12
Variables and intervals selected studied in the DOE

| | Low Setting | Center Setting | High Setting |
|---|---|---|---|
| | (−1) | (0) | (+1) |
| X1: Filler ratio | 0.25 | 0.50 | 0.75 |
| Cellulose/Mannitol | | | |
| X2: Disintegrant type | Crospovidone | Croscarmellose | Sodium Starch |
| | | Sodium | Glycolate |
| X3: % Disintegrant | 2 | 6 | 10 |
| X4: % Glidant | 0.50 | 1.25 | 2.00 |
| (Aerosil200) | | | |
| X5: Lubricant type | Magnesium | Sodium Stearyl | Sodium Stearyl |
| | stearate | Fumarate (SSF) | Fumarate |
| | (MgSt) | | |
| X6: % Lubricant | 0.50 | 1.0 | 1.5 |

TABLE 13
List of experimental conditions

| | X1: | | | | X5: | |
|---|---|---|---|---|---|---|
| | Filler | | X3: % | X4: % | Lub | X6: % |
| Run | ratio | X2: SD type | SD | Glidant | type | Lub |
| 1¹ | 0.50 | Croscarmellose | 6 | 1.25 | SSF | 1.0 |
| 2 | 0.75 | Crospovidone | 2 | 1.25 | MgSt | 1.5 |
| 3 | 0.75 | SSGlycolate | 6 | 0.50 | SSF | 1.5 |
| 4 | 0.50 | SSGlycolate | 2 | 0.50 | MgSt | 0.5 |
| 5¹ | 0.50 | Croscarmellose | 6 | 1.25 | SSF | 1.0 |
| 6 | 0.25 | SSGlycolate | 2 | 2.00 | SSF | 1.5 |
| 7 | 0.75 | SSGlycolate | 10 | 2.00 | MgSt | 1.0 |
| 8 | 0.75 | Croscarmellose | 2 | 2.00 | SSF | 0.5 |
| 9¹ | 0.50 | Croscarmellose | 6 | 1.25 | SSF | 1.0 |
| 10 | 0.50 | Crospovidone | 10 | 2.00 | SSF | 1.5 |
| 11 | 0.25 | Crospovidone | 2 | 0.50 | SSF | 1.0 |
| 12 | 0.25 | Croscarmellose | 10 | 0.50 | MgSt | 1.5 |
| 13¹ | 0.50 | Croscarmellose | 6 | 1.25 | SSF | 1.0 |
| 14 | 0.25 | Crospovidone | 6 | 2.00 | MgSt | 0.5 |
| 15 | 0.75 | Crospovidone | 10 | 0.50 | SSF | 0.5 |
| 16 | 0.25 | SSGlycolate | 10 | 1.25 | SSF | 0.5 |

¹center point

In order to estimate the influence of the factors on resulting final blends, physical properties were evaluated and compared (i.e. flowability, bulk density, Carr's index, Hausner's ratio). Finally, the final blends were compressed to understand the impact of the relevant factors on tablet core tensile strength, disintegration time and dissolution rate.

Table 14 lists the studied response variables

| Process step | Response variable |
|---|---|
| Final blending | Flowability |
| | Particle size distribution |
| | Bulk/Tapped density |
| | Carr-s index, Hausner ratio |
| Tableting | Disintegration time |
| | Dissolution rate |
| | Tensile strength |
| | Ejection force |

Table 14-1 and Table 14-2 list the detailed batch composition

TABLE 14-1

| | Material/Batch | | | | | | | | | | | |
|---|---|---|---|---|---|---|---|---|---|---|---|---|
| | F3-01/05/ | | | | | | | | | | | |
| | 09/13¹ | | F3-02 | | F3-03 | | F3-04 | | F3-06 | | F3-07 | |
| | % | mg | % | mg | % | mg | % | mg | % | mg | % | mg |
| Compound (A) | 20.00 | 50.00 | 20.00 | 50.00 | 20.00 | 50.00 | 20.00 | 50.00 | 20.00 | 50.00 | 20.00 | 50.00 |
| Copovidone | 10.00 | 25.00 | 10.00 | 25.00 | 10.00 | 25.00 | 10.00 | 25.00 | 10.00 | 25.00 | 10.00 | 25.00 |
| [Kollidon VA64] | | | | | | | | | | | | |
| Sodium Lauryl | 0.10 | 0.25 | 0.10 | 0.25 | 0.10 | 0.25 | 0.10 | 0.25 | 0.10 | 0.25 | 0.10 | 0.25 |
| Sulfate [Duponol C] | | | | | | | | | | | | |
| Mannitol SD200 | 19.90 | 49.75 | 19.90 | 49.75 | 19.90 | 49.75 | 19.90 | 49.75 | 19.90 | 49.75 | 19.90 | 49.75 |
| Total granules | 50.00 | 125.00 | 50.00 | 125.00 | 50.00 | 125.00 | 50.00 | 125.00 | 50.00 | 125.00 | 50.00 | 125.00 |
| Avicel PH102 | 20.88 | 52.19 | 33.94 | 84.84 | 31.50 | 78.75 | 23.50 | 58.75 | 11.13 | 27.81 | 27.75 | 69.38 |
| [Cellulose MK GR] | | | | | | | | | | | | |
| Mannitol DC | 20.88 | 52.19 | 11.31 | 28.28 | 10.50 | 26.25 | 23.50 | 58.75 | 33.38 | 83.44 | 9.25 | 23.13 |
| Croscarmellose | 6.00 | 15.00 | — | — | — | — | — | — | — | — | — | — |
| Sodium [Natrium- | | | | | | | | | | | | |
| CMC-XL] | | | | | | | | | | | | |
| Sodium Starch | — | — | — | — | 6.00 | 15.00 | 2.00 | 5.00 | 2.00 | 5.00 | 10.00 | 25.00 |
| Glycolate | | | | | | | | | | | | |
| [Na-Carboxy- | | | | | | | | | | | | |
| methy-Stärke] | | | | | | | | | | | | |
| Crospovidone | — | — | 2.00 | 5.00 | — | — | — | — | — | — | — | — |
| [Polyvinylpoly- | | | | | | | | | | | | |
| pyrrolidone XL] | | | | | | | | | | | | |
| Aerosil 200 PH | 1.25 | 3.13 | 1.25 | 3.13 | 0.50 | 1.25 | 0.50 | 1.25 | 2.00 | 5.00 | 2.00 | 5.00 |
| Magnesium stearate | — | — | 1.50 | 3.75 | — | — | 0.50 | 1.25 | — | — | 1.00 | 2.50 |
| Sodium Stearyl | 1.00 | 2.50 | — | — | 1.50 | 3.75 | — | — | 1.50 | 3.75 | — | — |
| Fumarate | | | | | | | | | | | | |
| Total final blends | 100.00 | 250.00 | 100.00 | 250.00 | 100.00 | 250.00 | 100.00 | 250.00 | 100.00 | 250.00 | 100.00 | 250.00 |

TABLE 14-2

| | Material/Batch | | | | | | | | | | | | | |
|---|---|---|---|---|---|---|---|---|---|---|---|---|---|---|
| | F3-08 | | F3-10 | | F3-11 | | F3-12 | | F3-14 | | F3-15 | | F3-16 | |
| | % | mg | % | mg | % | mg | % | mg | % | mg | % | mg | % | mg |
| Compound (A) | 20.00 | 50.00 | 20.00 | 50.00 | 20.00 | 50.00 | 20.00 | 50.00 | 20.00 | 50.00 | 20.00 | 50.00 | 20.00 | 50.00 |
| Copovidone | 10.00 | 25.00 | 10.00 | 25.00 | 10.00 | 25.00 | 10.00 | 25.00 | 10.00 | 25.00 | 10.00 | 25.00 | 10.00 | 25.00 |
| [Kollidon | | | | | | | | | | | | | | |
| VA64] | | | | | | | | | | | | | | |
| Sodium | 0.10 | 0.25 | 0.10 | 0.25 | 0.10 | 0.25 | 0.10 | 0.25 | 0.10 | 0.25 | 0.10 | 0.25 | 0.10 | 0.25 |
| Lauryl Sulfate | | | | | | | | | | | | | | |
| [Duponol C] | | | | | | | | | | | | | | |
| Mannitol | 19.90 | 49.75 | 19.90 | 49.75 | 19.90 | 49.75 | 19.90 | 49.75 | 19.90 | 49.75 | 19.90 | 49.75 | 19.90 | 49.75 |
| SD200 | | | | | | | | | | | | | | |
| Total granules | 50.00 | 125.00 | 50.00 | 125.00 | 50.00 | 125.00 | 50.00 | 125.00 | 50.00 | 125.00 | 50.00 | 125.00 | 50.00 | 125.00 |
| Avicel PH102 | 34.13 | 85.31 | 18.25 | 45.63 | 11.63 | 29.06 | 9.50 | 23.75 | 10.38 | 25.94 | 29.25 | 73.13 | 9.56 | 23.91 |
| [Cellulose | | | | | | | | | | | | | | |
| MK GR] | | | | | | | | | | | | | | |
| Mannitol DC | 11.38 | 28.44 | 18.25 | 45.63 | 34.88 | 87.19 | 28.50 | 71.25 | 31.13 | 77.81 | 9.75 | 24.38 | 28.69 | 71.72 |
| Croscarmellose | 2.00 | 5.00 | — | — | — | — | 10.00 | 25.00 | — | — | — | — | — | — |
| Sodium | | | | | | | | | | | | | | |
| [Natrium- | | | | | | | | | | | | | | |
| CMC-XL] | | | | | | | | | | | | | | |
| Sodium Starch | — | — | — | — | — | — | — | — | — | — | — | — | 10.00 | 25.00 |
| Glycolate | | | | | | | | | | | | | | |
| [Na-Carboxy- | | | | | | | | | | | | | | |
| methy-Stärke] | | | | | | | | | | | | | | |
| Crospovidone | — | — | 10.00 | 25.00 | 2.00 | 5.00 | — | — | 6.00 | 15.00 | 10.00 | 25.00 | — | — |
| [Polyvinylpoly- | | | | | | | | | | | | | | |
| pyrrolidone | | | | | | | | | | | | | | |
| XL] | | | | | | | | | | | | | | |
| Aerosil 200 PH | 2.00 | 5.00 | 2.00 | 5.00 | 0.50 | 1.25 | 0.50 | 1.25 | 2.00 | 5.00 | 0.50 | 1.25 | 1.25 | 3.13 |
| Magnesium | — | — | — | — | — | — | 1.50 | 3.75 | 0.50 | 1.25 | — | — | — | — |
| stearate | | | | | | | | | | | | | | |
| Sodium Stearyl | 0.50 | 1.25 | 1.50 | 3.75 | 1.00 | 2.50 | — | — | — | — | 0.50 | 1.25 | 0.50 | 1.25 |
| Fumarate | | | | | | | | | | | | | | |
| Total final | 100.00 | 250.00 | 100.00 | 250.00 | 100.00 | 250.00 | 100.00 | 250.00 | 100.00 | 250.00 | 100.00 | 250.00 | 100.00 | 250.00 |
| blends | | | | | | | | | | | | | | |

Such Formulations were manufactured according to the process below:

### Manufacturing Process of Compound (a) Wet Media Milling and Fluid Bed Spray Granulation

1. Dissolve copovidone into the water under stirring
2. Add sodium lauryl sulfate to the solution of step 1 and dissolve under stirring
3. Add compound (A) to the solution of step 2 and suspend under stirring
4. Perform wet-media milling with the suspension of step 3
5. Dissolve required amounts of sodium lauryl sulfate and copovidone in the additional purified water under stirring
6. Weigh required amount of wet media milled suspension from step 4 and add to the solution of copovidone and sodium lauryl sulfate in purified water from step 5 to complete the suspension for spray granulation
7. Load the fluidized bed dryer with the Mannitol SD200 carrier
8. Perform spray granulation by spraying entire amount of suspension for spray granulation from step 5 onto Mannitol SD200 carrier from step 7. Note that the nanosuspension has to be stirred for 5 minutes before to be sprayed.

### Manufacturing Process of Compound (a) Final Blend Preparation and Compression

9. Sieve the granule with a screen size of 0.8 mm
10. Sieve Avicel PH102, mannitol DS, super-disintegrant (i.e. sodium starch glycolate, crospovidone, croscarmellose sodium) with a screen size of 0.5 mm and add to the granule of step 9
11. Blend mixture from step 10
12. Sieve magnesium stearate with a screen size of 0.5 mm and add to blend of step 11
13. Blend mixture of step 12 on diffusion mixer
14. Compress the blend from step 13

### Evaluation of Final Blend Properties

### Final Blend Particle Size Distribution:

The fraction of particles on each mesh size of the CAMSIZER apparatus was determined. It was observed that the addition of 50% external excipients in the final blend caused a reduction in the amount of coarse particles.

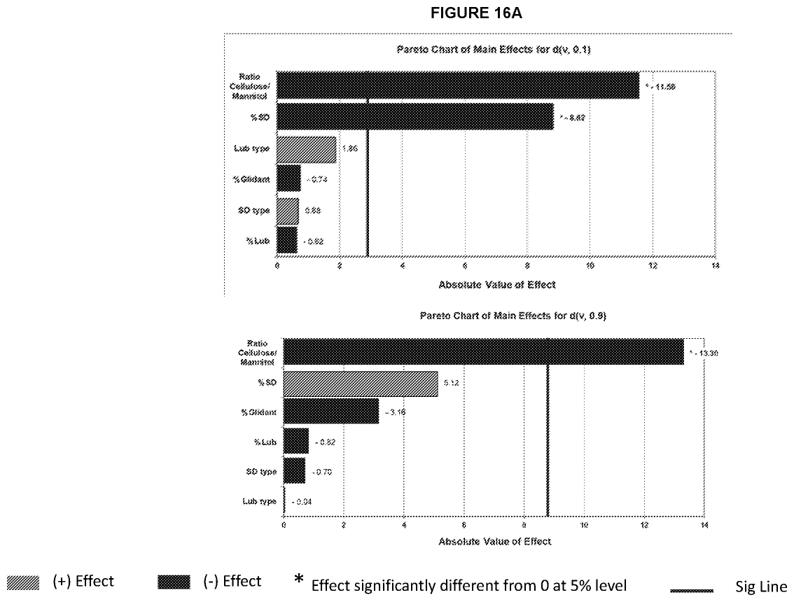

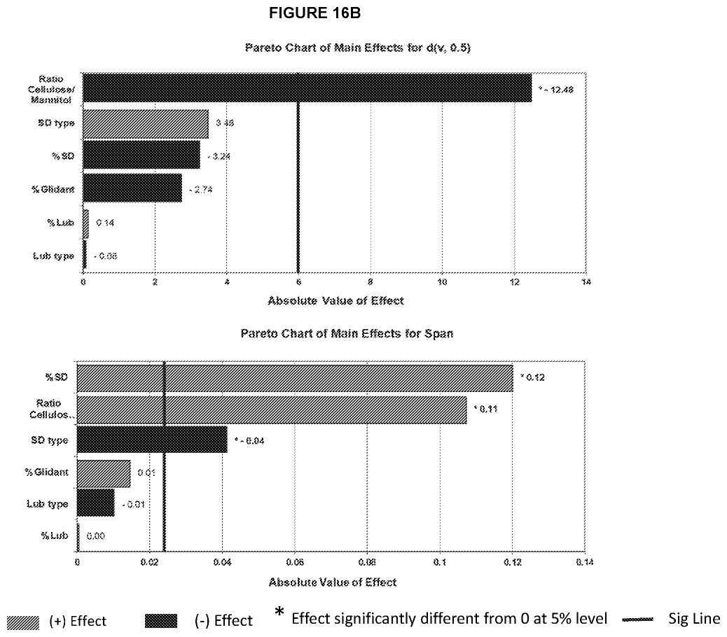

The pareto charts presented in FIGS. 16A and 16B show the six main effects from the study design plotted from highest to lowest effect, to see relative importance of effects to each other. It uses a + sign for positive effects (high level of factor gives higher response than low level of factor) and a − sign for negative effects (opposite direction). The significance line shows which effects are statistically significantly different from zero. In this case, the most influent factor for the final blend d10, d50 and d90 is the filler ratio cellulose/mannitol as significant. Meaning that using the high amount of mannitol (low filler ratio: 0.25) leads to coarser particle in the final blend. This graph shows also that some factors had impact on final blend span (i.e. level and type of SD, filler ratio).

### Final Blend Bulk and Tapped Density

The bulk and tapped density was obtained from the final blends of the 16 batches. It was observed that bulk and tapped densities are lower for batches containing high amount of mannitol corresponding to the ratio 0.25 (i.e. batches F3-06, F3-11, F3-12, F3-14 and F3-16) than batches containing high amount of MCC (filler ratio: 0.75, batches F3-02, F3-03, F3-07, F3-08 and F3-15).

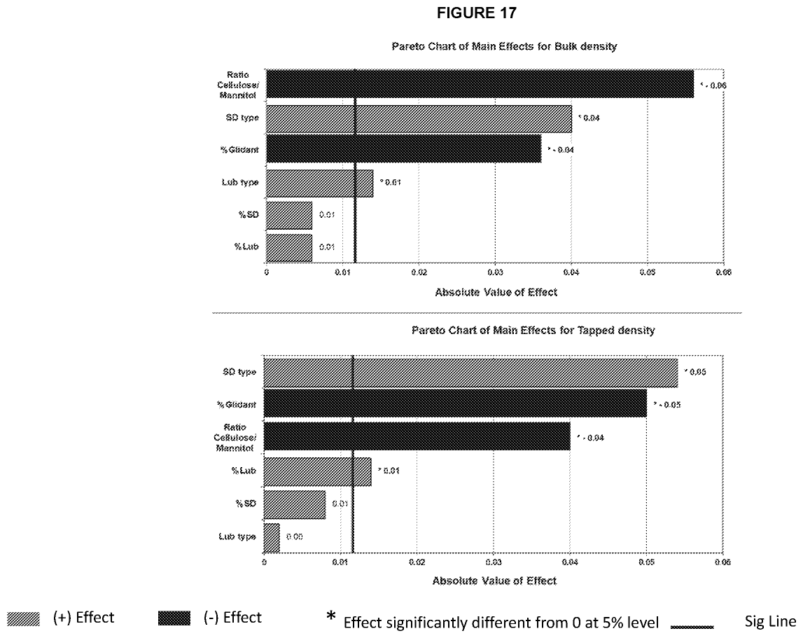

The pareto charts presented in FIG. 17 show the 3 most influencing factors that significantly impact the final blend bulk and tapped density. The 3 factors are the level of glidant, filler ratio and the disintegrant type.

### Final Blend Flow Properties:

Carr's Index and Hausner Ratio data give an indication on the theoretical flow properties of the 16 batches. The final blend behaviour was characterized with a revolution Powder Analyzer tester. This equipment can measure the powder's ability to flow by measuring the power, time and variances in energy in a rotating drum (diameter 100 mm at 0.6 rpm).

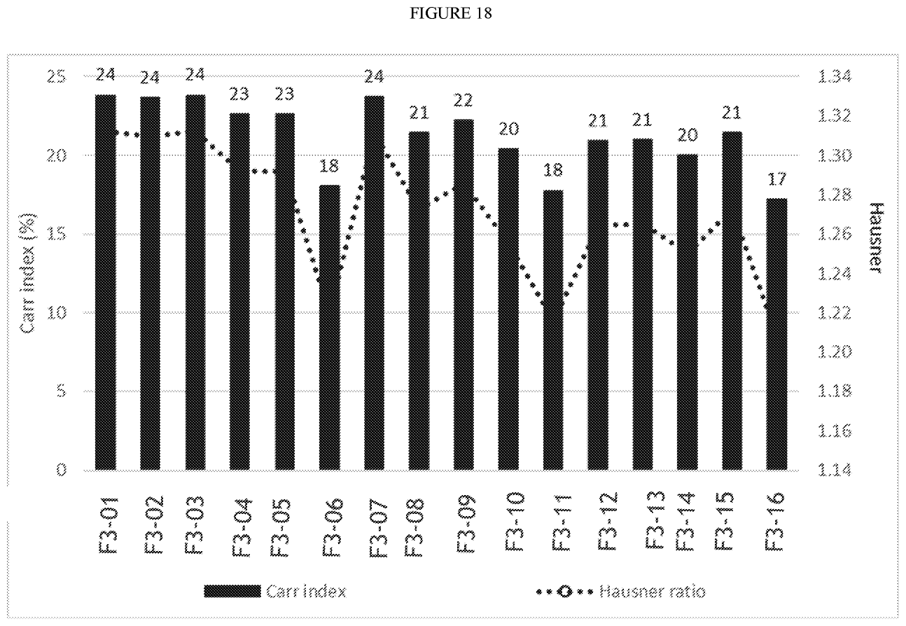

FIG. 18 indicates that all batches are similar and have passable theoretical flow properties according to the Pharmacopeia flowability scale (Carr's index below 25% and Hausner ratio 1.31).

The most influent factor for the final blend Carr's index and Hausner ratio is the filler ratio cellulose/mannitol. Meaning that using the high amount of mannitol leads to better flow properties.

It is seen that bulk density and flow characteristics are the final blend properties which are different between the batches. These differences are considered to be a result of the change in external phase composition (qualitative and quantitative). The particle size distribution (PSD) shows comparable values. Flowability results demonstrate better flow properties for batches containing a higher amount of mannitol corresponding to a filler ratio of 0.25 (e.g. batches F4-6, 11, 12 and 16).

### Evaluation of Tablet Core Properties

The 16 final blends were compressed with a 9 mm round flat punch tool using a power assisted single punch tablet press (KORSCH XP1). They were studied with regards to their compression behavior and were compared together.

### Compression Profile

In order to select the ideal compression force and hardness, a compression force-hardness profile was done prior start of compression run. For each batch, seven compression forces from 6 kN to 15 kN were assessed. The tablet crushing force (or hardness) was evaluated by using a hardness tester. And the tensile strength is commonly used to describe compact's degree of cohesion. The variations of the hardness and tensile strength under pressure are then drawn as a function of the main compression force.

The compression force-hardness profile was determined for the 16 batches. It was observed that tablet hardness increases with increasing compression force. The different compression force-hardness profiles are most likely due to the differences in external phase composition of the final blends (quantitative and qualitative). Indeed batch F3-15 shows the highest compression force-hardness profile while batch F3-14 shows the lowest compression force-hardness profile.

In order to evaluate the significance of these results, tensile strength profiles are drawn by using an equation (see below) to normalize values and to compare batches between batches. tensile strength profiles is determined as shown in the equation below and shows comprimability comparison. It shows the same trend as described for the compression.
Tensile strength,

$$σ TS = \frac{2 \cdot F}{π \cdot D \cdot t}$$

With: F, the crushing force (Hardness); D, the compact diameter and t, the compact thickness.

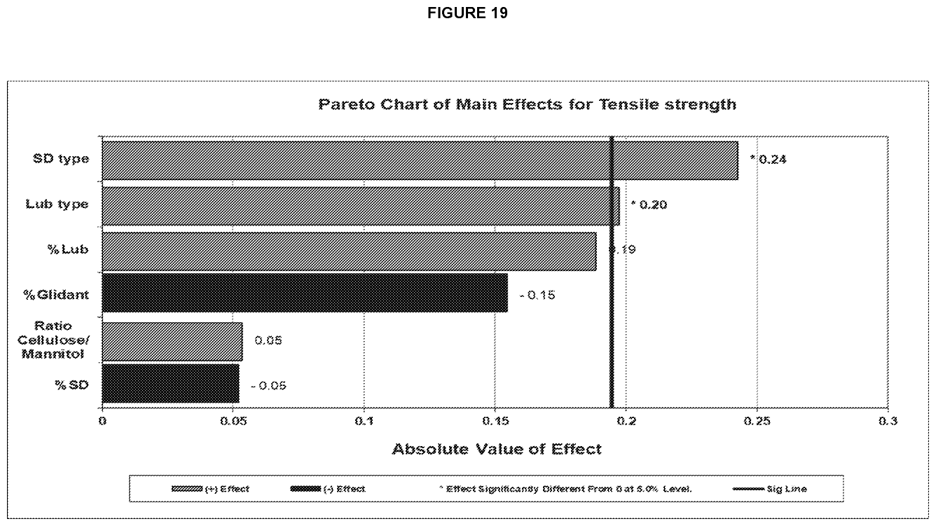

Tensile strength values taken for the pareto chart are coming from a tablet hardness of 90 N to compare all the batches together. 90 N tablet hardness was chosen based on the good balance between low friability results and acceptable disintegration time. The pareto chart (see FIG. 19) shows that the Super-Disintegrant (SD) type and the lubricant type are the two factors which are significantly impacting the tensile strength. Using SSF as lubricant and croscarmellose sodium as disintegrant, leads to a higher comprimability.

### Ejection Profile

The force necessary to eject the finished tablet is known as ejection force and can be used to quantify the sticking effect of a powder. This force can eject tablet by breaking tablet/die wall adhesion. Variation also occurs in ejection force when lubrication is inadequate and is also depending on tablet thickness. It is preferable to be as low as possible or less than 500 N.

The ejection force profile was recorded for all batches during compression cycle. It was observed that batch F3-14 presented the highest ejections force profiles (>800 N) close or far from the recommendation value of 500 N. The other profiles are low (>200 N). For more accuracy, specific ejection force is calculated by dividing the ejection force by the tablet weight and is expressed in N/g. The results showed the same trend as the ejection profile and could be divided into three groups:
High specific ejection profiles were recorded for the batch 0033-14 which presented specific ejection higher than 4000 N/g
Medium profiles were recorded for three batches 0033-04, 0033-08 and 0033-16 between 700 N/g and 2500 N/g
Low profiles were recorded for the other batches, which were below 600 N/g.

These differences can be explained by the difference in external phase composition.

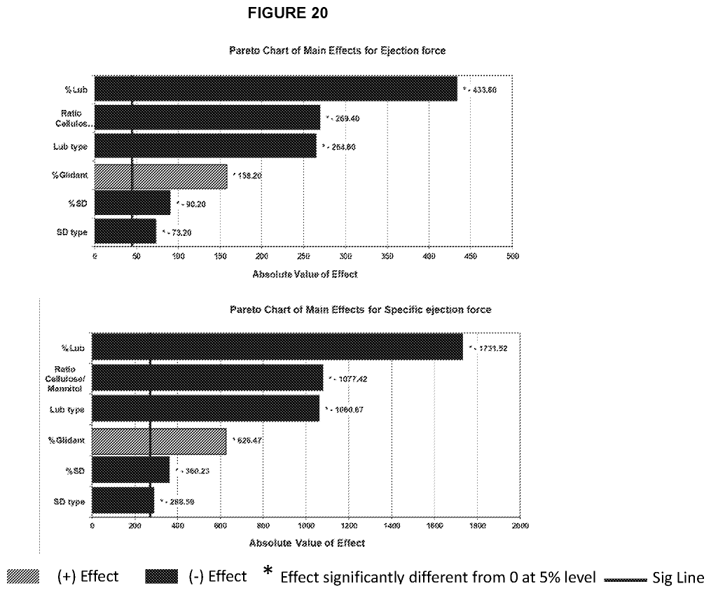

The two Pareto charts (FIG. 20) showed that four main contributing factors on ejection force and specific ejection force were the amount and the type of lubricant, ratio of filler and the amount of glidant.

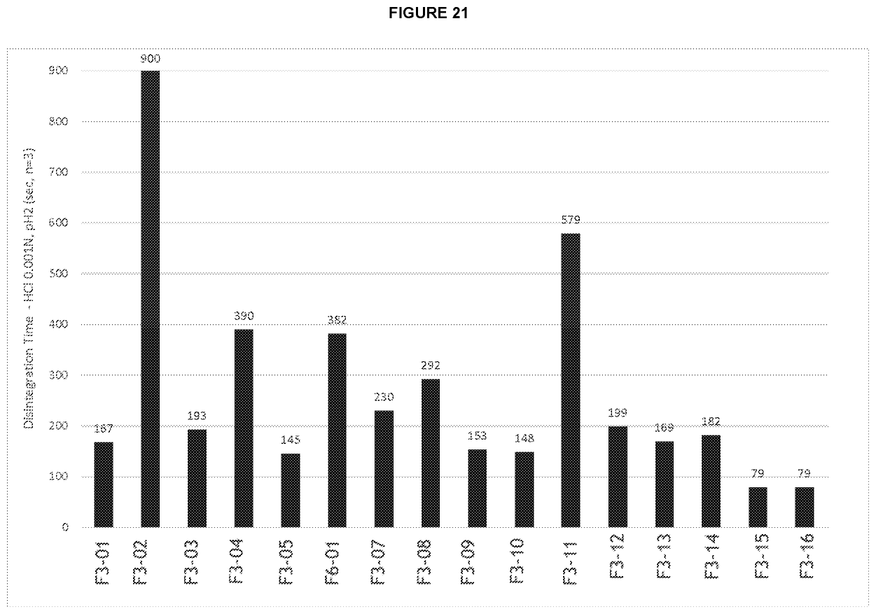

The amount and type of disintegrant was considered as negligible. The results showed that a well performing formulation is:
use of a minimum of 1% Sodium Stearyl Fumarate
low amount of glidant (below 1.25%)
low amount of mannitol (filler ratio up to 0.5).
   Disintegration Time (DT) of Tablet Cores Disintegration of tablet cores was performed in HCl, 0.01N pH2, representing the worst case medium for tablet of compound (A) disintegration, linked to an inherent gelling characteristic, as explained above. The disintegration time is expressed in maximum values of three tablet cores (see FIG. 21: maximum disintegration time values (90N)

Only batch F3-02 Shows higher disintegration times above 900 sec/15 min. All other batches never exceeded 600 sec/10 min but high variability between batches was observed most likely due to the differences in external phase composition. All six factors were found to have a significant impact on the maximum DT. However, regarding the magnitude of values, the ratio of fillers and the amount of glidant type may be considered as negligible. The four other factors are the main influencing factors. High amount of croscarmellose sodium (up to 6%) and high amount of SSFumarate (up to 1%) contribute to a faster tablet core disintegration time

The table 15 below summarizes the tablet core DT values based on the six factors and levels and includes means of low and high DT and means of DT for the centers (all 6 batches). Therefore, it can be concluded that the recommendations for fast tablet core DT values are:
Filler ratio: below 0.5%
Super-disintegrant (SD) type: SSGlycolate (DT: 159 sec) or Croscarmellose Sodium (DT: 255 sec)
disintegrant level: above 6%
Glidant level: about 1.25 (lowest DT with 1.25% of glidant used)
Lubricant type: SSFumarate
Lubricant level: below 1%

TABLE 15
Tablet core disintegration time based on factors and levels

| | X1: Filler | | | X4: % | | |
|---|---|---|---|---|---|---|
| | ratio | X2: SD type | X3: % SD | Glidant | X5: Lub type | X6: % Lub |
| Low/Center/ | 0.25/0.5/0.75 | Crosp/SSG/ | 2/6/10 | 0.5/1.25/2.0 | MgSt/SSF/ | 0.5/1.0/1.5 |
| High | | Crosca | | | SFF | |
| DT in pH 2 | 284-195-339 | 378-188-255 | 509-169-147 | 288-268-247 | 380-183-258 | 204-241-364 |

### Dissolution Profile of Tablet Cores

The dissolution rate (DR) of a tablet cores comprising compound (A) is measured by UV Spectroscopy in the automated equipment and performed in basket at a speed of 100 rpm in 0.01M HCl (pH2).

Batch F3-02 has the lowest dissolution profile, i.e. highest disintegration time observed for this batch. For all the other batches, more than 50% of compound (A) are dissolved in 30 minutes.

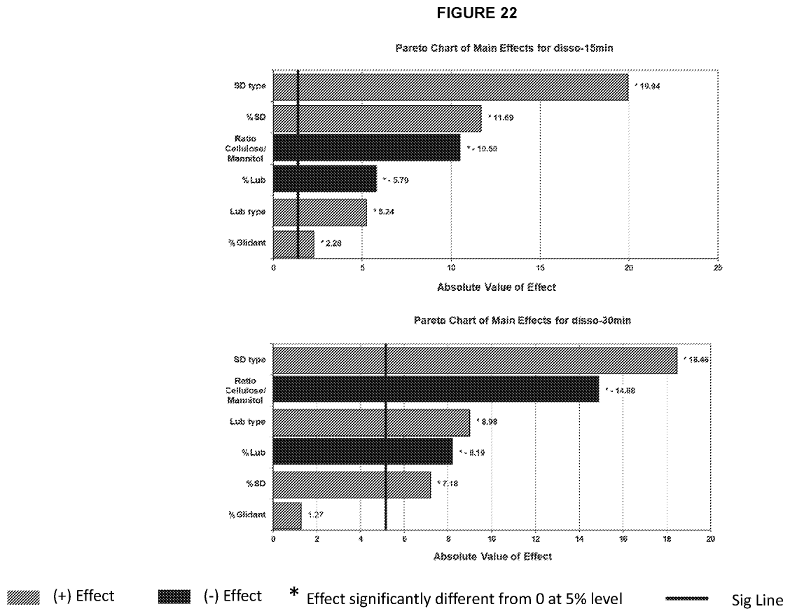

The Pareto graphs (FIG. 22) for tablet core dissolution rate at 15 min and 30 min show that all the 6 factors have a statistically significant impact on tablet dissolution rate at 15 min and 5 of the 6 factors have a statistically significant effect at 30 min.

Based on the tablet core dissolution rate at 15 min and 30 min at 15 min and 30 min, it was concluded that the recommendations for fast tablet core DR values are:
Filler ratio: below 0.5%
disintegrant type: SSGlycolate or Croscarmellose Sodium
disintegrant level: above 6%
Glidant level: no impact on DR
Lubricant type: SSFumarate
Lubricant level: below 1%

TABLE 16
Tablet core disintegration time based on factors and levels

| | X1: Filler | X2: SD | X3: % | X4: % | | |
|---|---|---|---|---|---|---|
| | ratio | type | SD | Glidant | X5: Lub type | X6: % Lub |
| Low/Center/ | 0.25/0.5/0.75 | Crosp/ | 2/6/10 | 0.5/1.25/2.0 | MgSt/SSF/ | 0.5/1.0/1.5 |
| High | | SSG/ | | | SFF | |
| | | Crosca | | | | |
| Dissolution | 49-45-39 | 33-46-53 | 37-46-49 | 45-40-48 | 41-45-46 | 45-47-39 |
| at 15 min | | | | | | |
| Dissolution | 68-60-53 | 51-61-69 | 56-61-63 | 63-55-64 | 56-60-65 | 63-63-54 |
| at 30 min | | | | | | |

### Conclusion on External Phase Composition Studies

Based on this statistical analysis this experiment reveals that the filler ratio is the main factor that impacts the final blend and tablet core properties. High level and type of super-disintegrant contributes to better disintegration time and dissolution rate. The level of glidant is the least influencing factor on the responses. The level and type of lubricant are significantly impacting the tablet core properties. The use of hydrophilic lubricant (i.e. SSFumarate) tends to decrease ejection force and to increase/improve the disintegration time and dissolution rate compared to magnesium stearate. Based on the studied experiment, the table 17 shows the most promising external phase composition which is the best suitable external phase for the formulation of compound (A), when used at an amount of 50% w/w of total composition weight.
Filler ratio: 0.5 is selected based on the good balance between high dissolution rate and low disintegration time
Super-disintegrant type and level: Sodium starch glycolate and Croscarmellose sodium
A minimum of 6% of disintegrant is required
The glidant level shows the lowest impact on tablet properties but can be used optionally, for example in a 1% amount.
Lubricant type: SSFumarate shows good DT and DR
A minimum of 1% of lubricant level is required for a better compression performance

TABLE 17
External phase composition

| X1: Filler | | X3: % | X4: % | X5: Lub | X6: % |
|---|---|---|---|---|---|
| ratio | X2: SD type | SD | Glidant | type | Lub |
| 0.50 | Croscarmellose | >6% | <1.25% | Sodium | >1% |
| | Sodium or sodium | | | stearyl | |
| | starch glycolate | | | fumarate | |

### Example 6: Further Studies on the External Phase (Amount)

The external phase studies in example 5 were limited to composition comprising an external phase in an amount of 50% w/w. For additional understanding of the external phase amount necessary to solve the gelling issue, the external phase amount was varied between 24% and 50% and a few more trials were done with different disintegrant types and variation of filler with cellulose Microcrystalline and Mannitol. No glidant was used in these trials as glidant has proved to be optional.

Tablet dosage forms were developed using formulation T1 comprising 20% w/w of Compound (A)) and formulation T2 comprising 25% w/w of Compound (A), as in Table 18. The tablet formulations depicted in Table 18 were prepared by mixing together the granule particles comprising Compound (A) and at least one pharmaceutically acceptable excipient, in a similar manner as the capsule formulation.

TABLE 18
Tablet formulation comprising Compound (A)

| | Experiments | | | | | | | | | |
|---|---|---|---|---|---|---|---|---|---|---|
| | 01 | 02 | 03 | 04 | 05 | 06 | 07 | 08 | 09 | 10 |
| | composition in mg/unit | | | | | | | | | |
| T1 | 250.0 | 250.0 | 250.0 | | | | 250.0 | 250.0 | 250.0 | 250.0 |
| Compound (A): 20% | | | | | | | | | | |
| Polyvinylpyrrolidone-vinyl | | | | | | | | | | |
| acetate copolymer 20% | | | | | | | | | | |
| Sodium lauryl sulfate 1% | | | | | | | | | | |
| Mannitol SD200 59% | | | | | | | | | | |
| T2 | | | | 200.0 | 200.0 | | | | | |
| Compound (A): 25% | | | | | | | | | | |
| Polyvinylpyrrolidone-vinyl | | | | | | | | | | |
| acetate copolymer 25% | | | | | | | | | | |
| Sodium lauryl sulfate 1.25% | | | | | | | | | | |
| Mannitol SD200 48.75% | | | | | | | | | | |
| External phase | | | | | | | | | | |
| Cellulose MK GR | 60.2 | | 126.0 | 176.0 | 110.2 | 110.2 | 220.0 | 126.0 | 126.0 | 126.0 |
| Mannitol SD200 | | 60.2 | | | | 200.0 | | | | |
| Crospovidone | 16.5 | 16.5 | 20.0 | 20.0 | 16.5 | 16.5 | 25.0 | | | 20.0 |
| Sodium starch glycolate | | | | | | | | 20.0 | | |
| Croscarmellose sodium | | | | | | | | | 20.0 | |
| Magnesium Stearate | 3.3 | 3.3 | 4.0 | 4.0 | 3.3 | 3.3 | 5.0 | 4.0 | 4.0 | 4.0 |
| | 330.0 | 330.0 | 400.0 | 400.0 | 330.0 | 330.0 | 500.0 | 400.0 | 400.0 | 400.0 |
| diameter (mm) | 10 | 10 | 11 | 11 | 10 | 10 | 11 | 11 | 11 | 11 |
| disintegration time water | 14-16 | 9-10 | 9-13 | 3-6 | 8-11 | <1 | 1 | 13-15 | 9-12 | 19-11 |
| (min) | | | | | | | | | | |
| disintegration time HCl | | >30 | 12-15 | 4-7 | 10-14 | <1 | 0.5-1 | >22 | 12-14 | 12-15 |
| pH 2.0 (min) | | | | | | | | | | |
| % of external phase | 24.2 | 24.2 | 37.5 | 50.0 | 39.4 | | 50.0 | 37.5 | 37.5 | 37.5 |

As mentioned in the present application, the problem with a formulation comprising compound (A) is that there is an inherent gelling behavior of Compound (A) at pH ≤2. This gelling behavior is influencing the disintegration time of the formulation (e.g. tablet) and therefore the disintegration time was measured in water, which is the standard test, and in addition in hydrochloric acid which has a pH=2.

All tested formulations in Table 18 had good disintegration behavior in water, and a difference was seen at pH=2. Fastest disintegration time in both media was achieved with an amount of 50% of pharmaceutically acceptable excipients in the external phase. Another factor for fast disintegration was found to be the amount of Compound (A) added onto the (a) inert substrate and the selection of the disintegrant type. In this first screening trials 1-ethenyl-2-pyrrolidinone homopolymer (commercially available under the name Crospovidone-CAS 9003-39-8) and Croscarmellose sodium achieved fastest disintegration time at pH=2.

An amount of 40% w/w of pharmaceutically acceptable excipients in the external phase in combination with a 20% w/w of Compound (A) in the granule was below 15 min disintegration time at pH 2 with the best performing disintegrants.

It was concluded that a minimum of 40% w/w of external phase is preferred.

Finally, in order to gain knowledge on chemical and physical stability, two variants were selected for a short stability program. Both variants were delivered as film coated tablets.
Compound (A)-F12-01 is the same composition as Compound (A)-F10-04
Compound (A)-F12-02 is the same composition as Compound (A)-F10-07

TABLE 19
stability samples

| Compound (A)-F12- | 01 | 02 |
|---|---|---|
| Compound (A), granule 20% | 200.0 | 250.0 |
| (0009-01)/25% (0009-03) | | |
| Compound (A) 20%/25% | | |
| Kollidon VA64 20%/25% | | |
| SLS 1%/1.25% | | |
| Mannitol SD200 59%/48.75% | | |
| Cellulose MK GR | 176.0 | 220.0 |
| Crospovidone | 20.0 | 25.0 |
| Magnesium Stearate | 4.0 | 5.0 |
| | 400.0 | 500.0 |
| diameter (mm) | 11 | 12 |
| thickness (mm) | 4.4 | 4.6 |
| hardness (N) | 80-85 | 95-105 |
| tensile strength | | |
| disintegration time water | 6-8 | 6-6.5 |
| (min) - core | | |
| disintegration time water | 8-9.5 | 5-7 |
| (min) - FCT | | |
| disintegration time HCl pH | 6.5-7 | 8-9.5 |
| 2.0 (min) - core | | |
| disintegration time HCl pH | 7-8 | 7-8.5 |
| 2.0 (min) - FCT | | |

The formulation comprising compound (A) as described above in table 19 can be described as very stable, no incompatibility of the drug substance with formulation composition was observed, even water uptake (expected for hygroscopic excipients present) during storage did not lead to observations during appearance testing.

### Example 7: Granule Quantitative and Qualitative Studies

An experiment was carried out to investigate the granule quantitative and qualitative composition. Four factors were selected for assessment and are listed in Table 20.

TABLE 20
Variables and intervals selected for the design of experiments

| | Low Setting | Center Setting | High Setting |
|---|---|---|---|
| Variables | (−1) | (0) | (+1) |
| A: Copovidone ratio | 0.3 | 0.5 | 0.7 |
| B: Sodium Lauryl Sulfate | 0.010 | 0.025 | 0.040 |
| ratio | | | |
| C: Mannitol ratio | 0 | 0.25 | 0.50 |
| D: Drug load [%] | 25 | 30 | 35 |

The granule composition is defined by the excipient ratios which are based on the amount of solid to be sprayed on the carrier surface to form a matrix. The excipient level is then defined by the equation below:

Excipient level=Drug load×Excipient ratio

The experiments will be performed using a 2nd order polynomial model (2⁴⁻¹ fractional factorial) including 4 center points as described in Table 21 resulting in a total of 12 experiments to be conducted.

TABLE 21
Listing of experiment

| Batch | A: | B: Sodium Lauryl | C: | D: Drug |
|---|---|---|---|---|
| number | Copovidone | Sulfate | Mannitol | Load |
| F6-01 | 0.7 | 0.010 | 0 | 35 |
| F6-02 | 0.3 | 0.040 | 0 | 35 |
| F6-03 | 0.3 | 0.040 | 0.50 | 25 |
| F6-04 | 0.7 | 0.010 | 0.50 | 25 |
| F6-05 | 0.7 | 0.040 | 0.50 | 35 |
| F6-06 | 0.3 | 0.010 | 0 | 25 |
| F6-07 | 0.3 | 0.010 | 0.50 | 35 |
| F6-08 | 0.7 | 0.040 | 0 | 25 |
| F6-09 | 0.5 | 0.025 | 0.25 | 30 |
| F6-010 | 0.5 | 0.025 | 0.25 | 30 |
| F6-011 | 0.5 | 0.025 | 0.25 | 30 |
| F6-012 | 0.5 | 0.025 | 0.25 | 30 |

The four replicate center points were used as the experimental error to test all 4 main effects, 3 sets of confounded pairs of 2-ways interactions. In order to estimate the influence of the factors on resulting granules and, final blends, the respective physical properties were evaluated and compared (i.e. flowability, bulk density, Carr's index, Hausner's ratio). Finally, the final blends were compressed to understand the impact of the relevant factors on tablet core tensile strength, disintegration time and dissolution rate.

A response variable is the observed response of an experiment consequent to the induced change of a process/formulation variable Table 22 lists the studied response variables.

TABLE 22
list of response variables

| Process step/Unit Operation | Response variable |
|---|---|
| Granule | Particle size distribution |
| | True/Bulk/Tapped density |
| | Flowability |
| | Resuspendability |
| | Dissolution rate |
| | Assay |
| | Compressibility/Porosity |
| | Tabletability (Tensile strength) |
| | Ejection force |
| Final Blending | Flowability |
| | Particle size distribution |
| | True/Bulk/Tapped density |
| | Carr index, Hausner ratio |
| | Segregation |
| Tableting | Disintegration time |
| | Dissolution rate |
| | Compressibility/Porosity |
| | Tabletability (Tensile strength) |
| | Ejection force |

The 12 tablet core batches were manufactured according to the proposed design of experiments. Table 23-1 and table 23-2 and table 23-3 present a summary of the 12 batch compositions with a granule batch size about 250 g. the manufacturing process was as described in example 6.

TABLE 23-1

| | batch number | | | | | |
|---|---|---|---|---|---|---|
| | F7-01 | | F7-02 | | F7-03 | |
| | Granule batch number | | | | | |
| | F6-01 | | F6-02 | | F6-03 | |
| | % | mg | % | mg | % | mg |
| Compound (A) (free base) | 17.50 | 50.00 | 17.50 | 50.00 | 12.50 | 50.00 |
| Copovidone [Kollidon VA64] | 12.25 | 35.00 | 5.25 | 15.00 | 3.75 | 15.00 |
| Sodium Lauryl Sulfate [Duponol C] | 0.18 | 0.50 | 0.70 | 2.00 | 0.50 | 2.00 |
| Mannitol SD200 | 0.00 | 0.00 | 0.00 | 0.00 | 6.25 | 25.00 |
| Mannitol SD200 (carrier) | 20.08 | 57.36 | 26.55 | 75.86 | 27.00 | 108.00 |
| Total granules | 50.00 | 142.86 | 50.00 | 142.86 | 50.00 | 200.00 |
| Avicel PH102 [Cellulose MK GR] | 21.00 | 60.00 | 21.00 | 60.00 | 21.00 | 84.00 |
| Mannitol DC | 21.00 | 60.00 | 21.00 | 60.00 | 21.00 | 84.00 |
| Croscarmellose Sodium | 6.00 | 17.14 | 6.00 | 17.14 | 6.00 | 24.00 |
| [Natrium-CMC-XL] | | | | | | |
| Aerosil 200 PH | 1.00 | 2.86 | 1.00 | 2.86 | 1.00 | 4.00 |
| Sodium Stearyl Fumarate | 1.00 | 2.86 | 1.00 | 2.86 | 1.00 | 4.00 |
| Total final blends | 100.00 | 285.71 | 100.00 | 285.71 | 100.00 | 400.00 |

TABLE 23-2

| | batch number | | | | | |
|---|---|---|---|---|---|---|
| | F7-04 | | F7-05 | | F7-06 | |
| | Granule batch number | | | | | |
| | F6-04 | | F6-05 | | F6-06 | |
| | % | mg | % | % | % | mg |
| Compound (A) (free base) | 12.50 | 50.00 | 12.50 | 12.50 | 12.50 | 50.00 |
| Copovidone [Kollidon VA64] | 8.75 | 35.00 | 3.75 | 3.75 | 8.75 | 35.00 |
| Sodium Lauryl Sulfate [Duponol C] | 0.13 | 0.50 | 0.13 | 0.13 | 0.13 | 0.50 |
| Mannitol SD200 | 6.25 | 25.00 | 0.00 | 0.00 | 6.25 | 25.00 |
| Mannitol SD200 (carrier) | 22.38 | 89.50 | 33.63 | 33.63 | 22.38 | 89.50 |
| Total granules | 50.00 | 200.00 | 50.00 | 50.00 | 50.00 | 200.00 |
| Avicel PH102 [Cellulose MK GR] | 21.00 | 84.00 | 21.00 | 21.00 | 21.00 | 84.00 |
| Mannitol DC | 21.00 | 84.00 | 21.00 | 21.00 | 21.00 | 84.00 |
| Croscarmellose Sodium | 6.00 | 24.00 | 6.00 | 6.00 | 6.00 | 24.00 |
| [Natrium-CMC-XL] | | | | | | |
| Aerosil 200 PH | 1.00 | 4.00 | 1.00 | 1.00 | 1.00 | 4.00 |
| Sodium Stearyl Fumarate | 1.00 | 4.00 | 1.00 | 1.00 | 1.00 | 4.00 |
| Total final blends | 100.00 | 400.00 | 100.00 | 400.00 | 100.00 | 400.00 |

TABLE 23-3

| | batch number | | | | | |
|---|---|---|---|---|---|---|
| | | | | | F7-09^{1,} F7-10^{1,} | |
| | F7-07 | | F7-08 | | F7-11^{1,} F7-12¹ | |
| | Granule batch number | | | | | |
| | | | | | F6-09^{1,} F6-10^{1,} | |
| | F607 | | F6-08 | | F6-11^{1,} F6-12¹ | |
| | % | mg | % | mg | % | mg |
| Compound (A) (free base) | 17.50 | 50.00 | 12.50 | 50.00 | 15.00 | 50.00 |
| Copovidone [Kollidon VA64] | 5.25 | 15.00 | 8.75 | 35.00 | 7.50 | 25.00 |
| Sodium Lauryl Sulfate [Duponol C] | 0.18 | 0.50 | 0.50 | 2.00 | 0.38 | 1.25 |
| Mannitol SD200 | 8.75 | 25.00 | 0.00 | 0.00 | 3.75 | 12.50 |
| Mannitol SD200 (carrier) | 18.33 | 52.36 | 28.25 | 113.00 | 23.38 | 77.92 |
| Total granules | 50.00 | 142.86 | 50.00 | 200.00 | 50.00 | 166.67 |
| Avicel PH102 [Cellulose MK GR] | 21.00 | 60.00 | 21.00 | 84.00 | 21.00 | 70.00 |
| Mannitol DC | 21.00 | 60.00 | 21.00 | 84.00 | 21.00 | 70.00 |
| Croscarmellose Sodium | 6.00 | 17.14 | 6.00 | 24.00 | 6.00 | 20.00 |
| [Natrium-CMC-XL] | | | | | | |
| Aerosil 200 PH | 1.00 | 2.86 | 1.00 | 4.00 | 1.00 | 3.33 |
| Sodium Stearyl Fumarate | 1.00 | 2.86 | 1.00 | 4.00 | 1.00 | 3.33 |
| Total final blends | 100.00 | 285.71 | 100.00 | 400.00 | 100.00 | 333.33 |

¹Center point

### Granule Scanning Electron Microscopy (SEM)

The granules were visualized and analyzed with respect to their shapes, surface morphology and roughness.

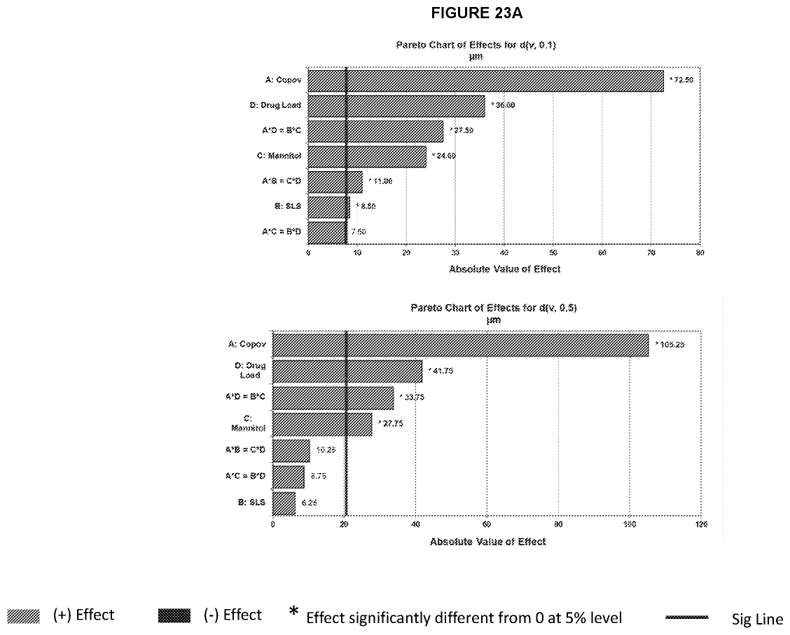

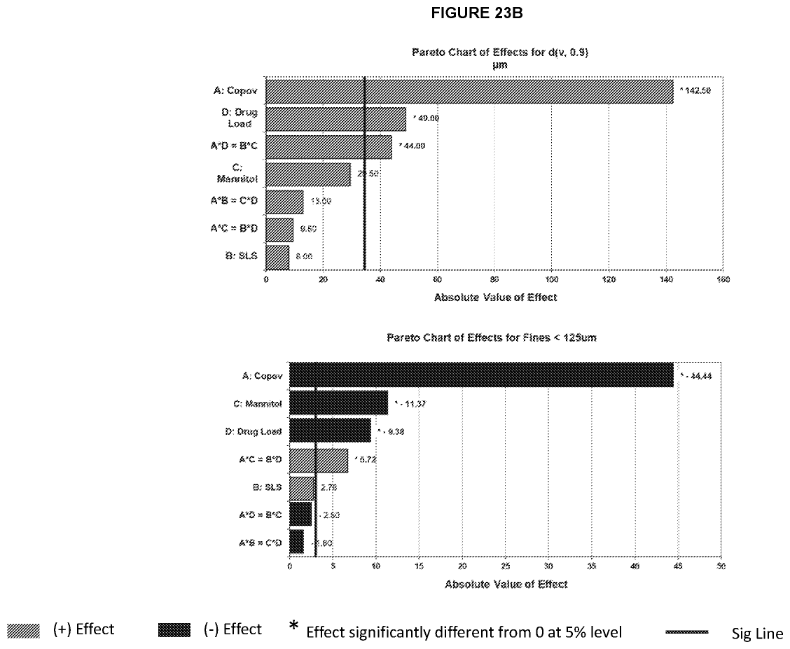

It was observed that batches (F6-01-04-05-08 and the four center points F6-09-10-11-12) containing a copovidone ratio up to 0.5 consist of coarser particles with a d50 >250 μm. Agglomeration between granules can be observed from the SEM images. The Pareto chart in FIGS. 23A and 23B summarizes various effects. The level of copovidone is shown as significantly impacting the granule PSD (particle size distribution). High amount of copovidone leads to coarser granule particles.

### Granule Bulk and Tapped Density

Bulk and tapped density data were obtained from the sieved granules of the 12 batches as determined in example 6.

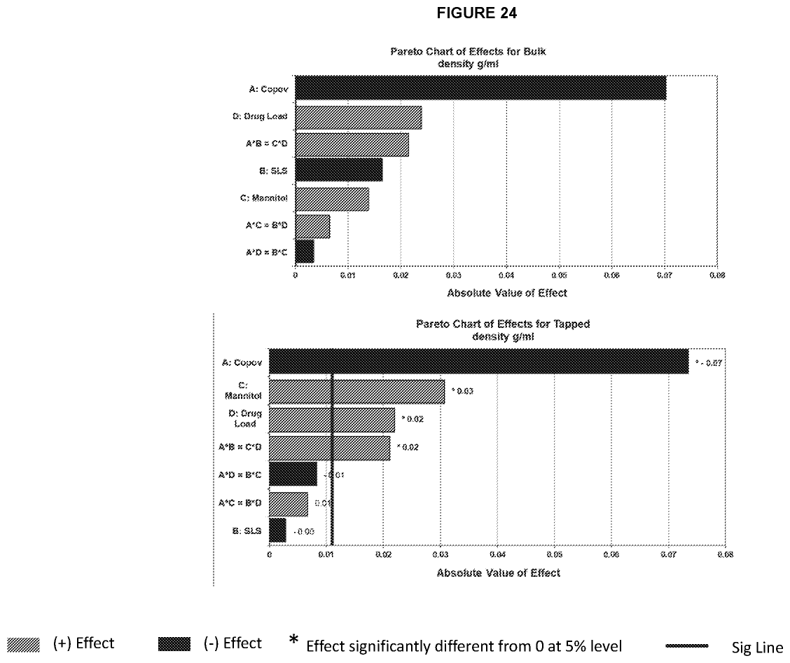

It was observed that bulk and tapped densities are higher for batches containing low amount of copovidone (i.e. F6-02-03-06-07). The pareto chart presented in FIG. 24 showed the most influencing factor that significantly impacts the granules tapped density, is the copovidone (from 0.50 g/ml to 0.57 g/ml).

### Granule Flow Characteristics (Granule Carr's Index and Hausner Ratio)

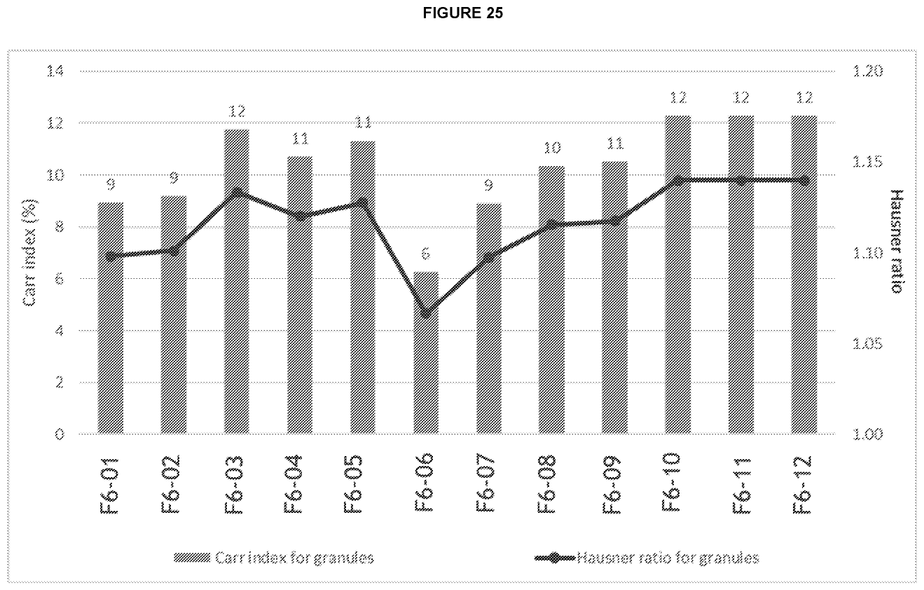

Granules Carr's Index and Hausner Ratio data give an indication on the theoretical flow properties of the 12 batches. FIG. 25 indicates that all batches are similar and have good/excellent theoretical flow properties according to the Pharmacopeia flowability scale (Carr's index below 15% and Hausner ratio below 1.18).

### Granule Flowability

The granule behavior was characterized with a revolution Powder Analyzer tester. This equipment can measure the powder's ability to flow by measuring the power, time and variances in energy in a rotating drum (diameter 100 mm at 0.6 rpm). The results from avalanche median (between 2.2 sec and 3.0 sec) and the avalanche angle results (between 37° and 42°) show passable/good flow properties of all 12 granule batches. All avalanche power results (<18 cch) and surface linearity results (0.99%) show good flow properties.

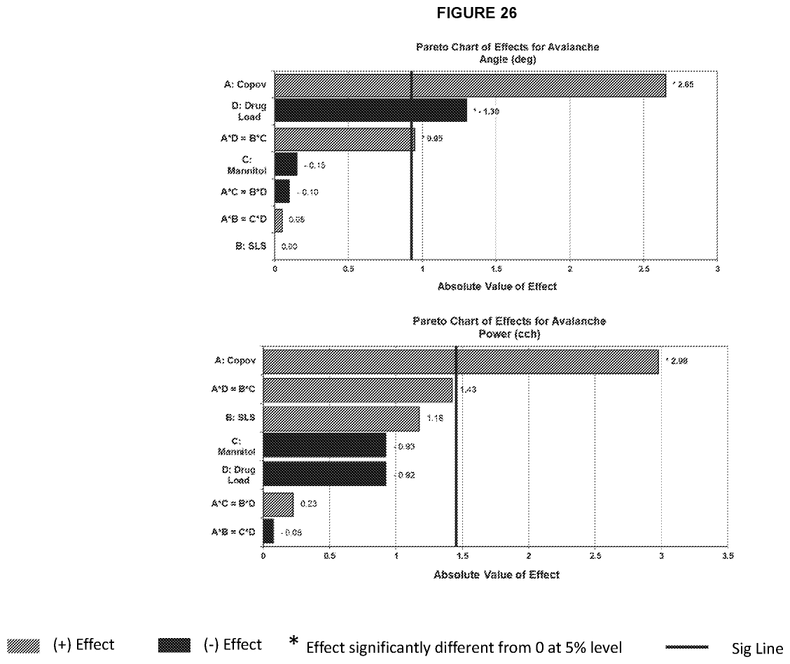

The pareto charts from the FIG. 26 showed that copovidone has a significant impact on granule flowability. In the study, high level of copovidone leads to coarser particles and better flow behavior of the granules.

### Granule Assay and Resuspendability

The granule assay and granule resuspendability of the 12 batches are listed in the Table 24. 95±2% of drug substance were measured for all granules. No compensation was applied during spray granulation.

The granules were subjected to reconstitution/resuspendability by PSD using photon correlation spectroscopy (PCS). Photon Correlation Spectroscopy (PCS) is used to size particles from below 5 nm to several microns. This technique operates on the principle that particles move randomly in gas or liquid. The particle size of the DS in wet media milled before dilution for spray granulation is 123 nm.

TABLE 24
Granule assay and resuspendability

| Batch | | Assay | Resuspendability |
|---|---|---|---|
| numbers | Coding | value [%] | [nm] |
| F6-01 | C.7-SLS.01-M0-DL35 | 93.3 | 134.6 |
| F6-02 | C.3-SLS.04-M0-DL35 | 95.5 | 186.2 |
| F6-03 | C.3-SLS.04-M.5-DL25 | 94.1 | 161.1 |
| F6-04 | C.7-SLS.01-M.5-DL25 | 95.0 | 134.2 |
| F6-05 | C.7-SLS.04-M.5-DL35 | 95.5 | 134.4 |
| F6-06 | C.3-SLS.01-M0-DL25 | 95.3 | 213.6 |
| F6-07 | C.3-SLS.01-M.5-DL35 | 96.1 | 190.8 |
| F6-08 | C.7-SLS.04-M0-DL25 | 96.4 | 133.6 |
| F6-09 | C.5-SLS.025-M.25-DL30 | 96.1 | 139.0 |
| F6-10 | C.5-SLS.025-M.25-DL30 | 96.6 | 138.0 |
| F6-11 | C.5-SLS.025-M.25-DL30 | 96.7 | 137.7 |
| F6-12 | C.5-SLS.025-M.25-DL30 | 96.0 | 138.6 |

The results show that copovidone and SLS have a significant impact on granule resuspendability.

### Granule Compression Behavior

The compression behavior of 12 granule batches was characterized in order to gain knowledge on the product. Therefore, the granules were compressed with a 11.28 mm round flat punch tool using a power assisted single punch tablet press (Styl'One).

Granule compressibility: Compressibility is the powder's ability to deform under pressure. During powder densification, the porosity of a powder bed decreases. The densification can be studied by monitoring porosity under load. The tablet porosity is calculated after ejection by measuring the tablet's dimensions (i.e. thickness, diameter), weight and density. It was observed that porosity decreases with higher compression forces. All batches show a porosity below 8% at 25 MPa compression force. The 4 center points presented the highest porosity profiles compared to the other batches.

Granule tabletability: Tabletability is the ability to form mechanically strong compacts. Different tests are be performed, like compression force-hardness profiles and tensile strength profiles. A compression force-hardness profile was done for each batch. Five compression forces from 5 kN to 45 kN were assessed. The tablet crushing force (or hardness) was evaluated by using a hardness tester. Tensile strength is commonly used to describe compact's degree of cohesion. The variations of the hardness and tensile strength under pressure are then displayed as a function of the main compression force.

It was observed that tablet hardness increases with increased compression force. Different compression behaviors were observed between batches and low variability for each compression force. The batch F6-01 shows a decreasing hardness 25 kN compression force. The three granules F6-05, 07 and 09 show a plateau 25 kN. The 4 centers points presented the lowest and similar compression force-hardness profiles. The different compression force-hardness profiles are most likely linked to the differences in granules phase composition as expected. In order to evaluate the significance of these results, tensile strength profiles are drawn to normalize values and to compare batches between batches. All granules presented high tabletability and same trend as the compression force-hardness profiles.

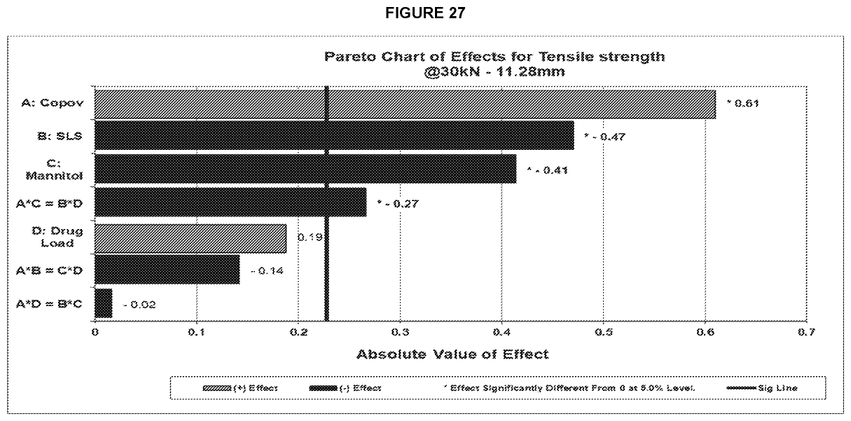

Tensile strength values taken for the pareto chart below are coming from the tablets compressed at 25-30 kN. The pareto chart (FIG. 27) shows that the level of copovidone, SLS and mannitol are the 3 factors which are significantly impacting the tensile strength. High amount of copovidone, low amount of SLS and no mannitol in the granule composition lead to a higher tabletability.

Granule compressibility: It was observed that the tensile strength of compacted granules decreases with higher porosity. Similar compactability profile was observed for all granule batches, as the compacts show a tensile strength about 2 MPa at 20% porosity.

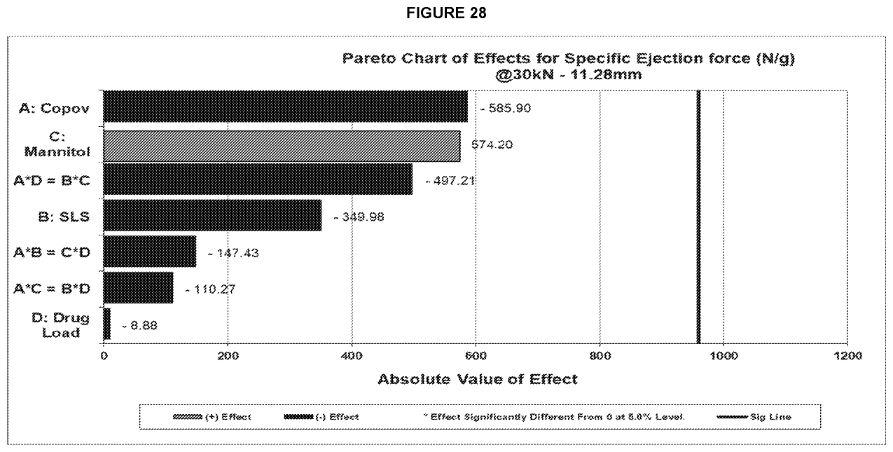

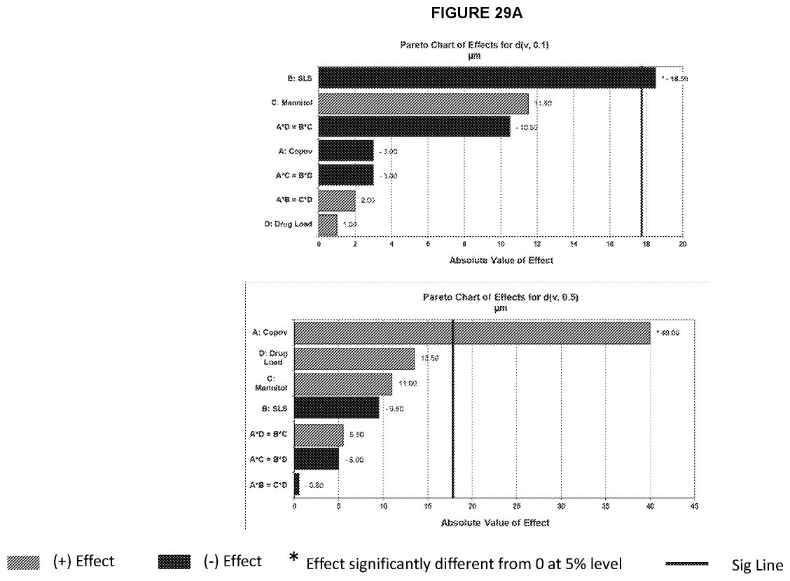

Granule ejection profile: The ejection force profiles were recorded for all batches during compression cycle. For more accuracy, specific ejection force was calculated by dividing the ejection force by the tablet weight and is expressed in N/g. FIG. 28 shows the various influencing factors of the granule composition on the specific ejection profile.

### Evaluation of Final Blend Properties

The characterization of the twelve final blends was performed and the results are summarized in Table 25-1 and Table 25-2 and detailed further in the following subsections.

TABLE 25-1
Summary of final blend properties

| | Batch | | | | | |
|---|---|---|---|---|---|---|
| | F7-01 | F-02 | F7-03 | F7-04 | F7-05 | F7-06 |
| | Coding | | | | | |
| | C.3- | C.7- | C.5- | C.5- | C.5- | C.5- |
| | SLS.01- | SLS.04- | SLS.025- | SLS.025- | SLS.025- | SLS.025- |
| | M.5-DL35 | M0-DL25 | M.25-DL30 | M.25-DL30 | M.25-DL30 | M.25-DL30 |
| d(v, 0.1) - μm | 63 | 55 | 58 | 81 | 55 | 64 |
| d(v, 0.5) - μm | 212 | 152 | 160 | 199 | 208 | 153 |
| d(v, 0.9) - μm | 337 | 219 | 231 | 301 | 360 | 224 |
| Span - μm | 1.3 | 1.1 | 1.1 | 1.1 | 1.5 | 1.0 |
| Fines ≤ 125 μm - % | 39 | 71 | 64 | 41 | 43 | 69 |
| True density - g/cm³ | 1.40 | 1.43 | 1.44 | 1.42 | 1.40 | 1.44 |
| Bulk density - g/ml | 0.49 | 0.50 | 0.50 | 0.48 | 0.51 | 0.49 |
| Tapped density - g/ml | 0.59 | 0.60 | 0.60 | 0.57 | 0.62 | 0.58 |
| Carr index | 17 | 16 | 16 | 16 | 17 | 15 |
| Hausner ratio | 1.21 | 1.19 | 1.19 | 1.19 | 1.21 | 1.18 |
| Flow behavior¹ | Fair | Fair | Fair | Fair | Fair | Good |
| Avalanche | 2.8 | 3.0 | 3.1 | 3.0 | 2.9 | 2.9 |
| median (sec) | | | | | | |
| Avalanche | 11.7 | 13.4 | 14.2 | 15.8 | 14.0 | 14.8 |
| power (cch) | | | | | | |
| Avalanche | 43.0 | 43.7 | 44.2 | 47.2 | 44.2 | 47.8 |
| angle (deg) | | | | | | |
| Avalanche | 1.0 | 1.0 | 1.0 | 1.0 | 1.0 | 1.0 |
| linearity (%) | | | | | | |

¹Pharmacopeia flowability scale

TABLE 25-2
Summary of final blend properties

| | Batch | | | | | |
|---|---|---|---|---|---|---|
| | F7-07 | F7-08 | F7-09 | F7-10 | F7-11 | F7-12 |
| | Coding | | | | | |
| | C.3- | C.7- | C.5- | C.5- | C.5- | C.5- |
| | SLS.01- | SLS.04- | SLS.025- | SLS.025- | SLS.025- | SLS.025- |
| | M.5-DL35 | M0-DL25 | M.25-DL30 | M.25-DL30 | M.25-DL30 | M.25-DL30 |
| d(v, 0.1) - μm | 90 | 56 | 58 | 76 | 61 | 66 |
| d(v, 0.5) - μm | 177 | 183 | 205 | 211 | 192 | 203 |
| d(v, 0.9) - μm | 244 | 281 | 329 | 320 | 297 | 311 |
| Span - μm | 0.9 | 1.2 | 1.3 | 1.2 | 1.2 | 1.2 |
| Fines ≤ 125 μm - % | 53 | 49 | 41 | 37 | 45 | 40 |
| True density - g/cm³ | 1.43 | 1.42 | 1.42 | 1.42 | 1.42 | 1.42 |
| Bulk density - g/ml | 0.54 | 0.53 | 0.47 | 0.48 | 0.47 | 0.48 |
| Tapped density - g/ml | 0.62 | 0.55 | 0.57 | 0.58 | 0.57 | 0.57 |
| Carr index | 13 | 4 | 18 | 16 | 17 | 16 |
| Hausner ratio | 1.15 | 1.04 | 1.21 | 1.20 | 1.20 | 1.19 |
| Flow behavior¹ | Good | Excellent | Fair | Fair | Fair | Fair |
| Avalanche | 1.7 | 3.0 | 3.0 | 2.8 | 3.0 | 2.8 |
| median (sec) | | | | | | |
| Avalanche | 7.6 | 14.5 | 14.1 | 13.3 | 13.3 | 12.3 |
| power (cch) | | | | | | |
| Avalanche | 38.2 | 45.9 | 44.6 | 43.9 | 44.0 | 43.6 |
| angle (deg) | | | | | | |
| Avalanche | 1.0 | 1.0 | 1.0 | 1.0 | 1.0 | 1.0 |
| linearity (%) | | | | | | |

¹Pharmacopeia flowability scale

### Final Blend Particle Size

### Final Blend Particle Size Distribution

As shown in the table above the addition of 50% external phase excipients in the granules caused a reduction in the amount of coarse particles.

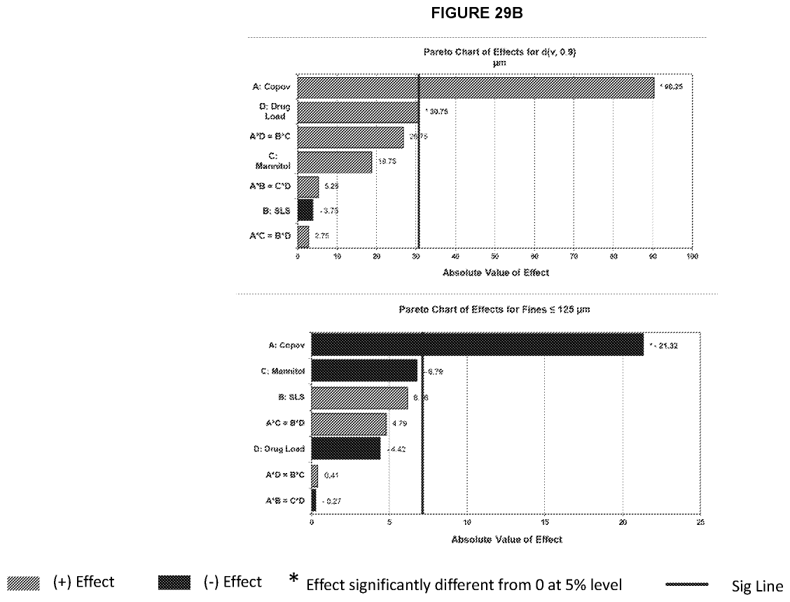

The pareto charts presented in FIGS. 29A and 29B show that the copovidone level is the most influencing factor for the final blend d50, d90 and fines particles below 125 μm. Same trend is observed for granules: high amount of copovidone leads to coarser particles. On the other hand, low copovidone significantly leads to high amount of fines.

### Final Blend Bulk and Tapped Density

According to summary table above, it was observed that bulk and tapped densities are similar between batches.

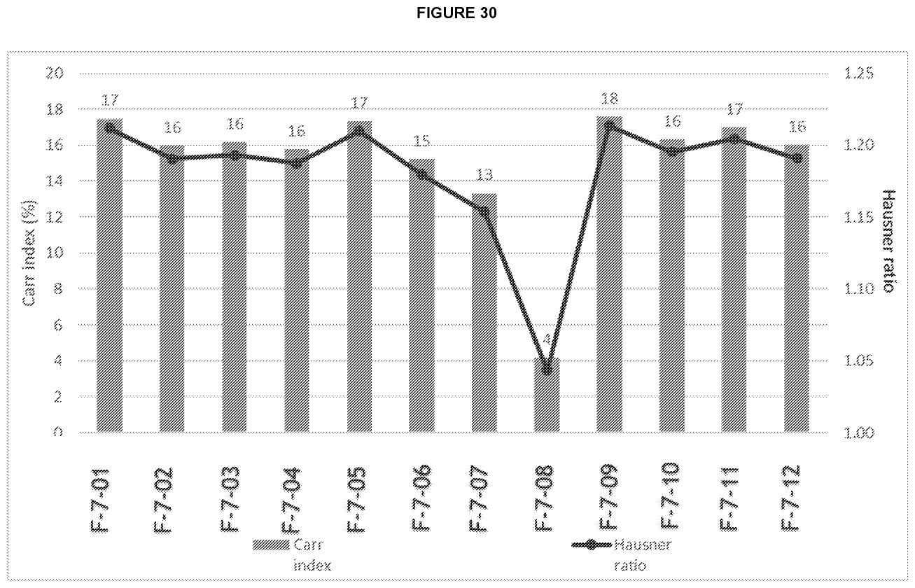

Carr's Index and Hausner Ratio: Carr's Index and Hausner Ratio data give an indication on the theoretical flow properties of the 12 batches. FIG. 30 indicates that all batches are similar and have good theoretical flow properties according to the Pharmacopeia flowability scale. The batch F7-08 shows an excellent flow property.

### Final Blend Flow Property

The final blend behavior was characterized with a revolution Powder Analyzer tester. This equipment can measure the powder's ability to flow by measuring the power, time and variances in energy in a rotating drum (diameter 100 mm at 0.6 rpm). The results from avalanche median (between 1.7 sec and 3.1 sec) and the avalanche angle results (between 38° and 48°) show passable/good flow properties of FB. All avalanche power results (<18 cch) and surface linearity results (>0.99%) show good flow properties.

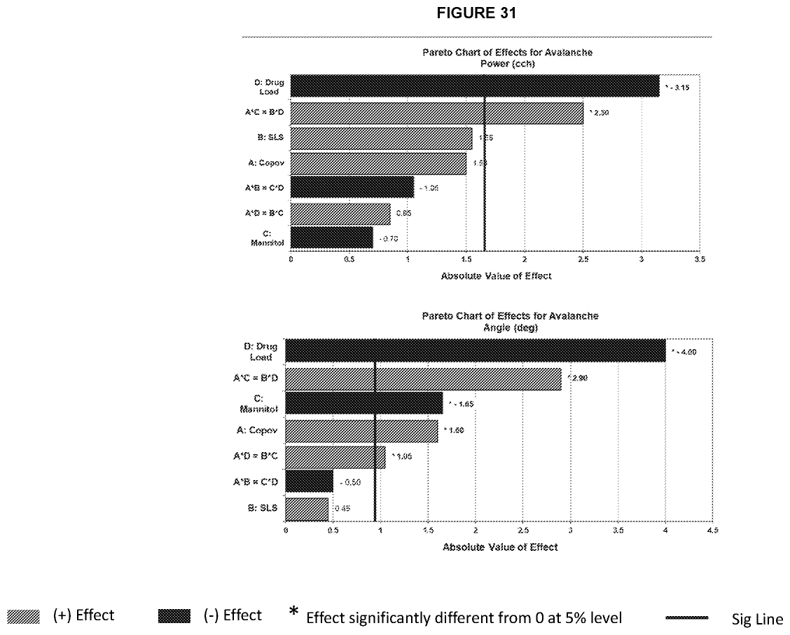

The pareto charts from the FIG. 31 shows that drug load significantly impacts the final blend flowability.

### Final Blend Flow Segregation Prediction

Segregation or demixing is the separation of components from a particulate mixture due to differences in physical characteristics (size, shape, density, etc.). There are several driving forces or mechanisms that can cause segregation. The most commonly occurring mechanism in the industry are sifting, fluidization and dusting. To limit segregation, material particle size distribution (PSD) should have the same distribution. For example, high difference in PSD between granules and excipients can separate physically the mixture and lead to segregation. Coarser particles may be entrained by gravity in the bottom and finer particles are located on the top of the blend. Depending on the powder behavior, the contrary can happen with coarse particles on the top and the fines on the bottom. The mixture can be distinctly divided. The external phase composition is about 50% w/w of the tablet weight (major amount for the two fillers: Avicel PH102 and Mannitol DC). This high amount of external phase could potentially lead to a separation between components due to differences in particle size.

To predict the potential segregation phenomenon, two methods were used:
1. particle size distribution comparison between material (i.e. granule, final blend, each excipients).
2. sieving segregation using different screen sieves

### Particle Size Distribution Comparison Method:

This study aims to compare the distribution of particle size of each final blend, granule and external phase excipients (i.e. Avicel PH102, Mannitol DC and croscarmellose sodium). Differences of particle sizes between inner phase (i.e. granules) and external phase could lead to segregation. Indeed, it is observed that the granules PSD is shifted towards the right direction, corresponding to coarse particles, while the external phase excipients (i.e. MCC and Mannitol) are shifted to the left, corresponding to finer particles. The ideal blend which could limit segregation phenomenon should have similar PSD curves. The batch F7-06 from this perspective has the most appropriate PSD. Batch F7-05 shows high segregation tendency.

### Sieving Segregation Method:

For the sieving segregation method, the powder mixture is added to a column of screen sieves in order to stress the powder to segregate by vibration (amplitude 1.0 mm, 5 min). The mixture is forced to separate into four fractions corresponding to the related screen sieves with fine particles on the bottom and coarse on top of the apparatus. The API content is then determined for each fraction in order to evaluate how the API is distributed throughout particle size fractions. Finally, the standard deviation is calculated to determine a potential segregation of the mixture. High standard deviation leads to high potential segregation. Only 3 granule batches were evaluated F6-01, F6-08 and F6-11 and their corresponding final blends F7-01, F7-08 and F7-11.

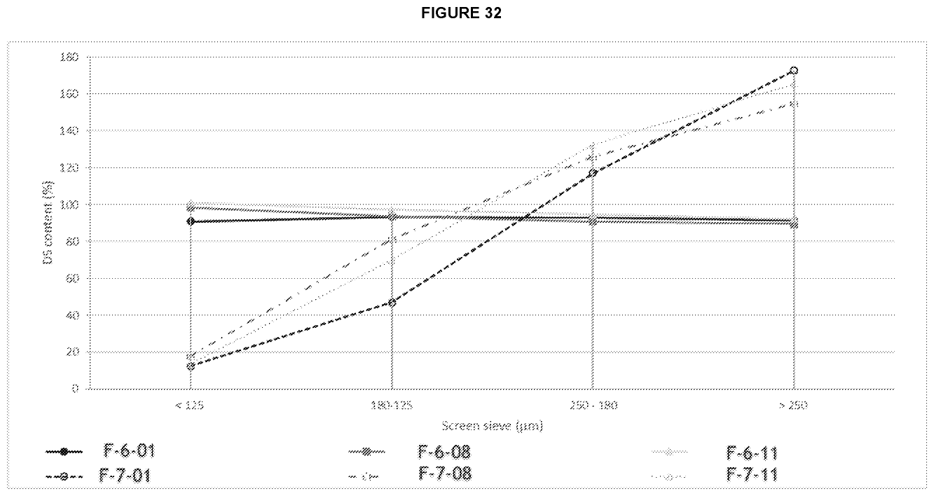

Table 26 summarizes the drug substance content measured in each fraction. The RSD value is used as a basis to compare the segregation between batches. The API is part of the granules and therefore not present in the external phase. The highest API content measured in the fraction form the top of the can be linked to the granules presented the coarser fraction. It was observed that the drug substance is homogeneously distributed in the granules for each fraction while, the final blends show higher potential for segregation with high RSD values (i.e. RSD from 63% to 82%). The batch F6-01 exhibits the highest RSD. This batch is proned to high segregation also seen from the high difference in PSD between granules and external phase (FIG. 32). Therefore, it can be concluded that the external phase level has an important impact on the drug product content uniformity. A good balance between the level of external phase and appropriate granules particle size distribution will be less prone to segregation.

TABLE 26
Granules and final blends segregation prediction by
sieving analysis (% compound (A) in each fraction)

| | F6-01 | F6-08 | F6-11 |
|---|---|---|---|
| | [C.3-SLS.01- | [C.7-SLS.04- | [C.5-SLS.025- |
| Batch | M.5-DL35] | M0-DL25] | M.25-DL30] |
| >250 μm | 91 | 99 | 101 |
| 250-180 | 93 | 94 | 97 |
| μm | | | |
| 180-125 | 93 | 91 | 95 |
| μm | | | |
| <125 μm | 91 | 90 | 92 |
| Total | 368 | 373 | 385 |
| Mean | 92 | 93 | 96 |
| StDev | 1 | 4 | 4 |
| RSD | 1 | 4 | 4 |
| | F7-01 | F7-08 | F7-11 |
| | [C.3-SLS.01- | [C.7-SLS.04- | [C.5-SLS.025- |
| Batch | M.5-DL35] | M0-DL25] | M.25-DL30] |
| >250 μm | 12 | 18 | 14 |
| 250-180 | 47 | 81 | 70 |
| μm | | | |
| 180-125 | 117 | 126 | 132 |
| μm | | | |
| <125 μm | 173 | 155 | 165 |
| Total | 349 | 380 | 381 |
| Mean | 87 | 95 | 95 |
| StDev | 72 | 60 | 67 |
| RSD | 82 | 63 | 71 |

### Final Blend Compression Behavior

The 12 final blends were compressed with a standard 11.28 mm round flat punch for compression characterization using a power assisted single punch tablet press (compaction simulator Styl'One Evolution). They were studied with regard to their compression behavior and results were compared.

Final blend compressibility: Compressibility is the powder's ability to deform under pressure. During powder densification, the porosity of a powder bed decreases. The densification can be studied by monitoring the porosity under load. The tablet porosity is calculated after ejection by measuring the tablet's dimensions (i.e. thickness, diameter), weight and density. It was observed that porosity decreases with increasing compression forces. All final blend batches show similar porosity profiles.

### Final Blend Tabletability

Tabletability is the ability to form mechanically strong compacts. Different tests was performed to study tabletability (i.e. compression force-hardness profile and tensile strength profile).

A compression force-hardness profile was done for each batch. Five compression forces from 5 kN to 45 kN were assessed. The tablet crushing strength (or hardness) was evaluated by using a hardness tester. The tensile strength is commonly used to describe compact's degree of cohesion. The variations of the hardness and tensile strength under pressure are then drawn as a function of the main compression force. It was observed that an increase of the compression force leads to higher tablet hardness. Different compression behaviors were observed between batches and variability was low. The batch F7-01 shows a decreasing hardness at 25 kN compression force. The granules from F7-06 show the highest tabletability profile and the batches F7-01 and F7-04 show the lowest tabletability profile. Compared to the granule tabletability profiles, no tendency for loss in hardness or plateau were observed for final blends. It was concluded that the external phase excipients have a positive impact on this property. Tensile strength profiles were recorded which allowed tabletability comparison. It showed that all tensile strength profiles of the final blends show similar trend compared to the compression force-hardness profiles with more accurate values using tensile strength.

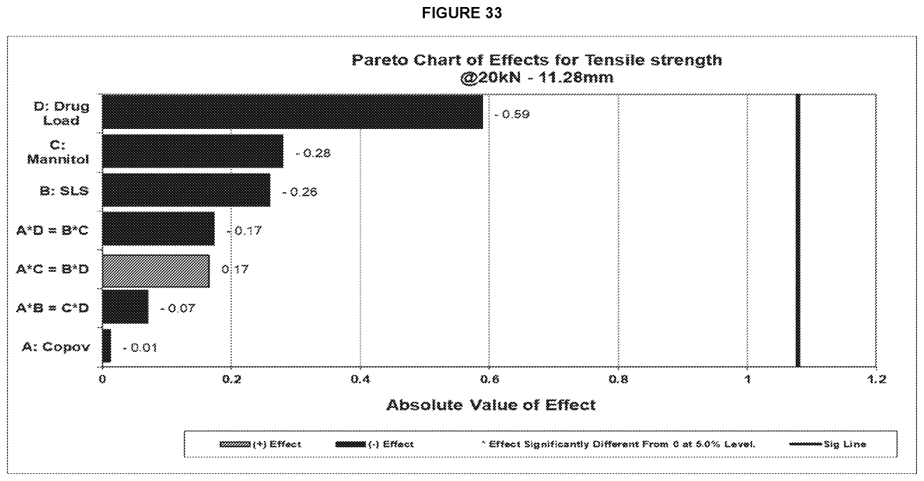

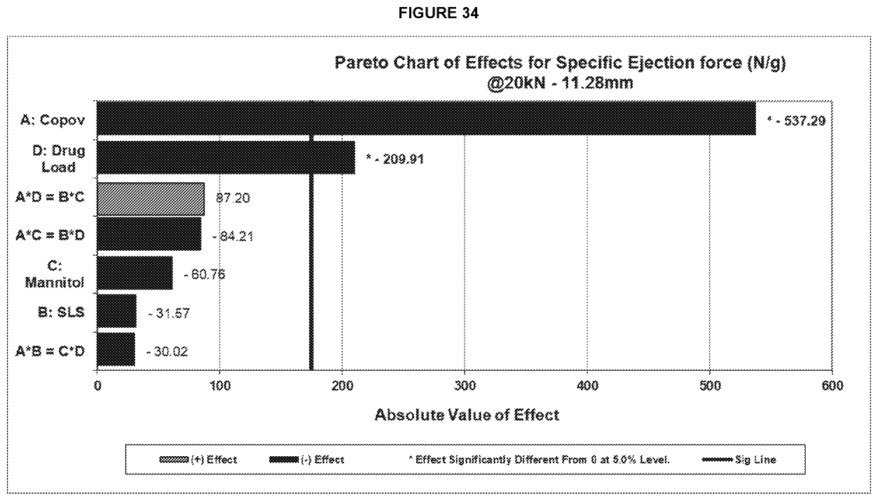

Tensile strength values taken for the pareto chart below are coming from the tablets compressed at 20 kN. The pareto chart (FIG. 33) shows that no factor has a significant impact on final blend tensile strength.

Final blend ejection profile: Ejection force profile was recorded for all batches during compression cycle. For more accuracy, specific ejection force is calculated by dividing the ejection force by the tablet weight and is expressed in N/g. The pareto chart (FIG. 34) shows that the main contributing factor on specific ejection force is the level of copovidone. High amount of copovidone leads to a low specific ejection force.

Evaluation of Tablet Core Properties at Tablet Hardness 90N and 120N with Adequate Punch

### Punch Tooling

The Table 27 summarized the tablet punch toolings used for the 50 mg dosage strengths with the 3 different drug loads, leading to different tablet weights (i.e. 25%, 35%, 40% granule drug load combined with 50% external phase).

TABLE 27
Punch tooling

| Granules drug load [%] | Punch tooling |
|---|---|
| 25 | Ø 10 mm NVR/984 |
| 30 | Ø 10 mm NVR/984 |
| 35 | Ø 11 mm NVR/984 |

### Tablet Core Ejection Forces

Table 28 presents the ejection force values recorded for tablet cores manufactured at 90 N and 120 N. It shows that for all batches at both hardness levels, the ejection forces are much lower compared to the recommend value of 500 N.

TABLE 28
Ejection forces values at tablet hardness of 90N and 120N

| Batch no: | At 90N tablet hardness [N] | At 120N tablet hardness [N] |
|---|---|---|
| F7-01 | 97 | 125 |
| F7-02 | 95 | 101 |
| F7-03 | 126 | 124 |
| F7-04 | 112 | 117 |
| F7-05 | 95 | 94 |
| F7-06 | 119 | 130 |
| F7-07 | 110 | 106 |
| F7-08 | 107 | 113 |
| F7-09 | 107 | 107 |
| F7-10 | 111 | 108 |
| F7-11 | 105 | 103 |
| F7-12 | 97 | 104 |

### Tablet Core Disintegration Time

Disintegration of tablet cores was performed in HCl, 0.01N pH 2 for both tablet core hardness levels (90 N and 120 N. For the 120 N tablet cores, the disintegration time in water was also measured. The disintegration time values were expressed as maximum values of three tablet cores (see Table 29). Only batch F7-07 shows higher disintegration times (DT) above 900 sec/15 min. All other batches never exceeded 480 sec/8 min. For batch F7-07, the DT was 4 times lower for the lower tablet hardness compared to tablets with higher tablet hardness.

TABLE 29
Tablet core disintegration time at 90N and
120N (maximum values expressed in seconds)

| | At 90N tablet hardness | At 120N tablet hardness | |
|---|---|---|---|
| Batch no: | pH 2 - HCl 0.01 | pH 2 - HCl 0.01 | Water |
| F7-01 | 438 | 446 | 312 |
| F7-02 | 117 | 259 | 290 |
| F7-03 | 122 | 206 | 249 |
| F7-04 | 157 | 268 | 290 |
| F7-05 | 1017 | 250 | 282 |
| F7-06 | 77 | 92 | 140 |
| F7-07 | 98 | 346 | 273 |
| F7-08 | 315 | 298 | 261 |
| F7-09 | 164 | 354 | 289 |
| F7-10 | 231 | 252 | 297 |
| F7-11 | 165 | 366 | 318 |
| F7-12 | 240 | 324 | 266 |

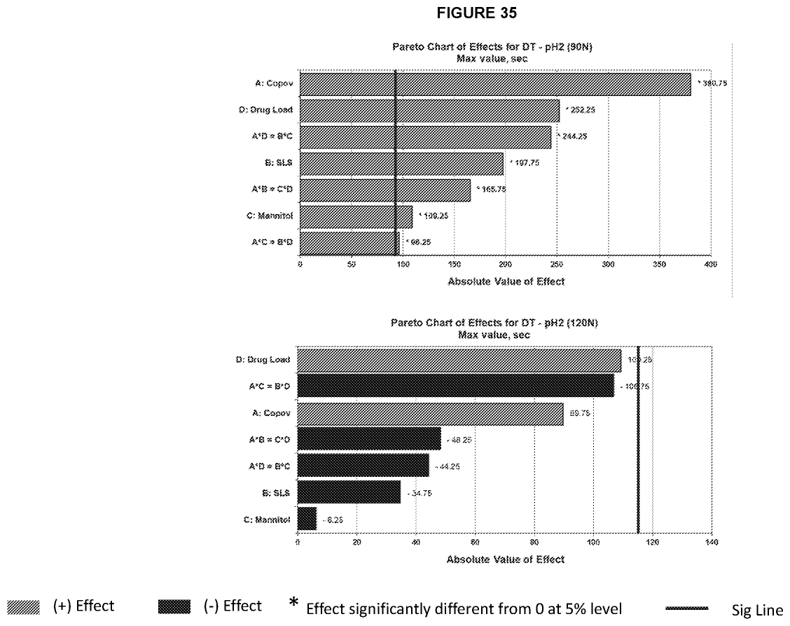

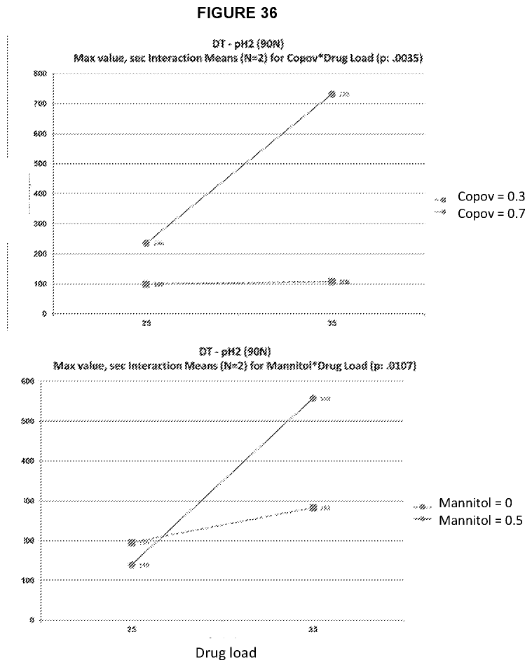

As shown in the pareto charts FIG. 35, all factors are significantly impacting the tablet core DT manufactured at 90 N and none is significantly impacting the tablet core DT with a higher tablet hardness at 120 N. The 2 main influencing factors are amount of copovidone and drug load. High amount of copovidone and high drug load lead to a higher DT for the 90 N tablet core hardness. It seems that tablet hardness has an important impact on the DT. The FIG. 36 shows the 2 way interactions on 90N tablet cores. It shows that high copovidone and the use of mannitol in the spray suspension lead to longer disintegration time.

### Dissolution Profile of Tablet Cores

The dissolution rate of tablet cores comprising compound (A) with 90N and 120N tablet hardness respectively, is measured by UV spectroscopy in the automated equipment and performed in paddle 50 rpm pH3 and basket at sped of 100 rpm in 0.01M HCl pH2. (conventional methods for dissolution test: Basket method according to Pharm. Eur. 2.9.3 “Dissolution Test for Solid Dosage Forms” or US pharmacopeia <711> “Dissolution” or Japanese pharmacopeia <6.10> “Dissolution Test”)

### Dissolution Profile of 90 N and 120 N Tablet Cores in Basket at a Speed of 100 Rpm(pH2)

Low variability was observed for all batches (RSD <5%) except for batch F7-07 with higher RSD values up to 5%. The 4 center point batches (i.e. F7-09-10-11-12) are reproducible and presented similar dissolution profiles.

The three batches with 90 N hardness: F7-01, F7-05 and F7-07 show the lowest dissolution profiles with basket at a speed of 100 rpm in 0.01M HCl pH2. This finding is supported by the highest disintegration time observed for these batches. For all the other batches, more than 80% of compound (A) was dissolved in 30 min but never reached 100% at 60 min. From 60 min to 75 min, the basket speed was increased from 100 rpm to 200 rpm.

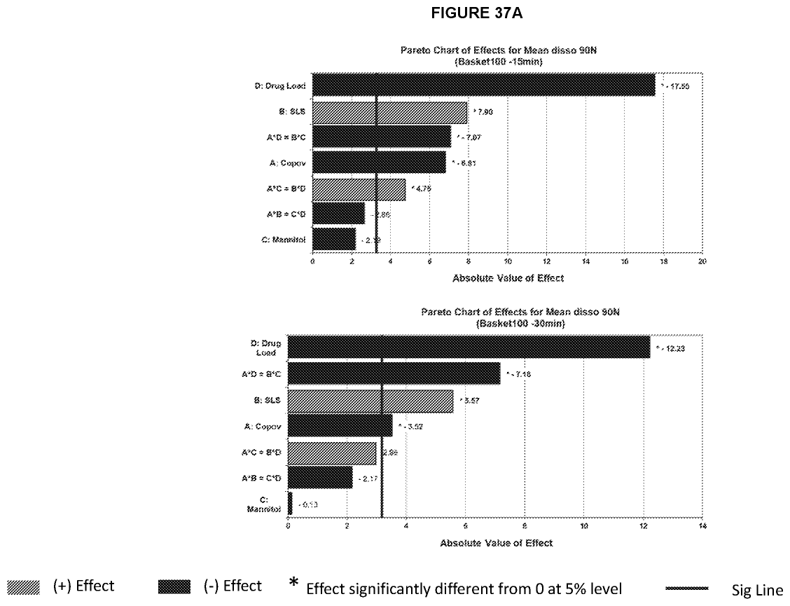

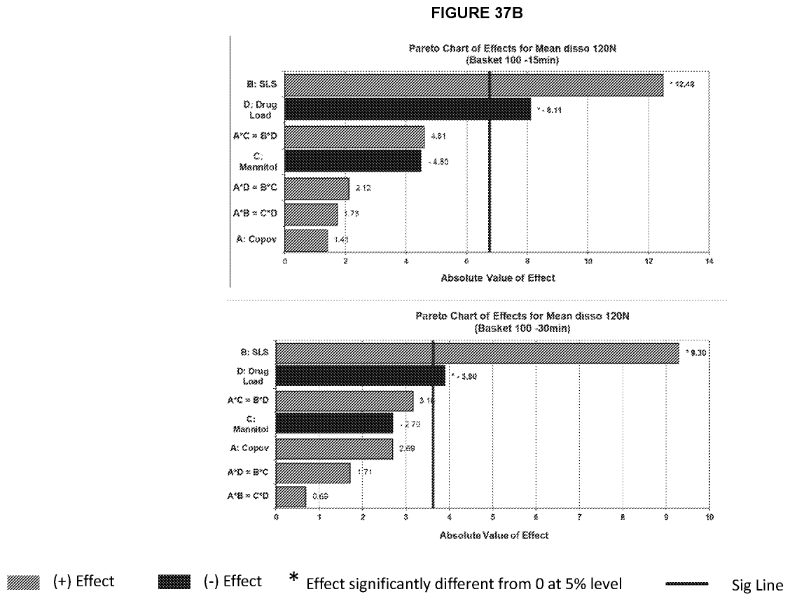

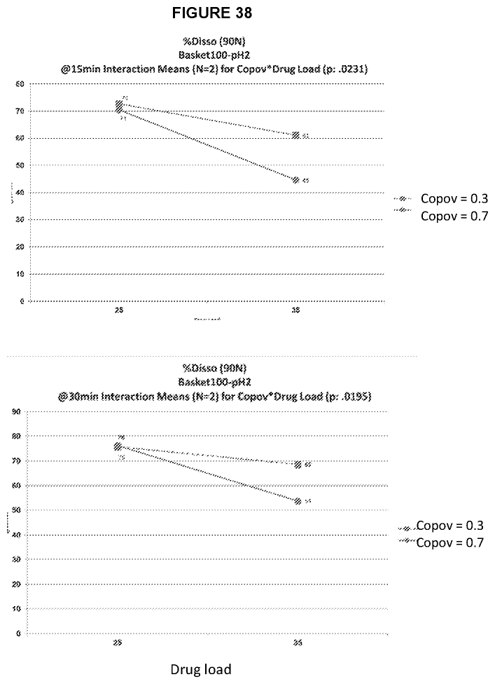

The pareto graphs (FIGS. 37A, 37B): FIG. 37A charts at 90N, FIG. 37B charts at 120N) for tablet core dissolution rate at 15 min and 30 min normalized to the tablet core assays show that the main significant contributing factors are the drug load and the amount of SLS. The recommendation to achieve fast dissolution rate profiles are the combination of low drug load and high amount of sodium lauryl sulfate. FIG. 38 shows 2 way interaction pareto graphs showing that low drug load and low copovidone lead to high dissolution rate for 90N tablet cores measured in Basket 100 rpm method.

Dissolution profile of 120 N tablet cores in paddle at a speed of 50 rpm (pH3) As previously mentioned, the drug substance (compound (A) is a Biopharmaceutics classification system Class 2 compound and is a weak base and exhibits strong pH dependent solubility (3 mg/mL at pH 1.2 and 0.003 mg/mL at pH 3). Dissolution rates of the 120 N tablet cores were assessed in pH3 with paddle at speed of 50 rpm in 0.001M HCl pH3 (900 mL). Low variability was observed for all batches (RSD <5%).

The Pareto graphs (FIG. 37) present the 120 N tablet core dissolution rate at 15 min and 30 min. Although, only slight differences between batches are observed, the Pareto graphs show that all the 4 factors have a significant impact on dissolution rate at 15 min in pH3 with paddle 50 rpm. At 30 min, the main influencing factor is the amount of SLS.

### Conclusion on all Experiments on the Granule Qualitative and Quantitative Composition.

The excipient ratios from the granule composition were based on the amount of solid to be sprayed on the carrier to form a matrix (i.e. copovidone, sodium lauryl sulfate, mannitol and drug load). The external composition is fixed at 50% for these experiment, as considered as good amount for tablet disintegration, dispersion and related dissolution rate.

Properties of granules, final blends (i.e. flowability, density, particle size distribution) and tablet cores (i.e. compactibility, disintegration time, dissolution rate) were evaluated. Table 30-1 and Table 30-2 summarize the main influencing factors that are statistically significant on granules, final blends and tablet cores responses.

TABLE 30-1
Summary of the most influencing factors for granule
responses (difference between high and low values)

| | A: | B: | C: | D: | | | |
|---|---|---|---|---|---|---|---|
| | Copovidone | SLS | Mannitol | Drug | A*B = | A*C = | A*D = |
| | ratio | ratio | ratio | load | C*D | B*D | B*C |
| Low Level: | 0.3 | 0.010 | 0.00 | 25 | n/a | n/a | n/a |
| High Level: | 0.7 | 0.040 | 0.50 | 35 | | | |
| Center Level: | 0.5 | 0.025 | 0.25 | 30 | | | |
| d10 | +73 | +8 | +24 | +36 | +11 | | +28 |
| d50 | +106 | | +28 | +42 | | | +34¹ |
| d90 | +143 | | | +49 | | | +44¹ |
| Fines < 125 μm | +45 | | −11 | −9 | | +7 | |
| Bulk density | | | | | | | |
| Tapped density | −0.07 | | +0.03 | +0.02 | +0.02 | | |
| Carr index | | | | | | | |
| Hausner ratio | | | | | | | |
| Avalanche Median | | | | | | | |
| Avalanche Power | +3 | | | | | | |
| Avalanche Angle | +3 | | | −1.3 | | | +1 |
| Avalanche linearity | | | | | | | |
| Resuspendability | −54 | −15 | −12 | | +14 | +12 | |
| Tensile strength | +0.6 | −0.5 | −0.4 | | | −0.3 | |
| at 30 kN | | | | | | | |
| Specific EF | | | | | | | |
| at 30 kN | | | | | | | |

TABLE 30-2
Summary of the most influencing factors for final blends and tablet
cores responses (difference between high and low values)

| | A: | B: | C: | D: | | | |
|---|---|---|---|---|---|---|---|
| | Copovidone | SLS | Mannitol | Drug | A*B = | A*C = | A*D = |
| | ratio | ratio | ratio | load | C*D | B*D | B*C |
| Low Level: | 0.3 | 0.010 | 0.00 | 25 | n/a | n/a | n/a |
| High Level: | 0.7 | 0.040 | 0.50 | 35 | | | |
| Center Level: | 0.5 | 0.025 | 0.25 | 30 | | | |
| d10 | | −19 | | | | | |
| d50 | +470 | | | | | | |
| d90 | +90 | | | | | | |
| Fines < 125 μm | −21 | | | | | | |
| Bulk density | | | | | | | |
| Tapped density | | | | | | | |
| Carr index | | | | | | | |
| Hausner ratio | | | | | | | |
| Avalanche Median | | | | | | | |
| Avalanche Power | | | | −3 | | +3 | |
| Avalanche Angle | +2 | | −2 | −4 | | +3 | +1 |
| Avalanche linearity | | | | | | | |
| Resuspendability | | | | | | | |
| Tensile strength | | | | | | | |
| at 20 kN | | | | | | | |
| Specific EF | −537 | | | −210 | | | |
| at 20 kN | | | | | | | |
| DT in pH 2 (90N) | +381 | +198 | −+109 | +252 | +166¹ | +96 | +244¹ |
| DT in pH 2 (120N) | | | | | | | |
| Average assay | −11 | | −5 | −10 | | | |
| tablet cores | | | | | | | |
| (90N and 120N) | | | | | | | |
| DR, 15 min (B100, | −7 | +8 | | −18 | | +5 | −7¹ |
| pH 2) - 90N | | | | | | | |
| DR, 30 min (B100, | −4 | +6 | | −12 | | | −7¹ |
| pH 2) - 90N | | | | | | | |
| DR, 15 min (B100, | | +13 | | −8 | | | |
| pH 2) - 120N | | | | | | | |
| Low Level: | 0.3 | 0.010 | 0.00 | 25 | n/a | n/a | n/a |
| High Level: | 0.7 | 0.040 | 0.50 | 35 | | | |
| Center Level: | 0.5 | 0.025 | 0.25 | 30 | | | |
| DR, 30 min (B100, | | +9 | | −4 | | | |
| pH 2) - 120N | | | | | | | |
| DR, 15 min (P50, | +11 | +3 | +5 | +3 | | −2 | +3 |
| pH 2) - 120N | | | | | | | |
| DR, 30 min (P50, | −2 | +9 | | | −3 | −7 | |
| pH 2) - 120N | | | | | | | |

Based on the statistical analysis, this experiment reveals that the ratio of copovidone, sodium lauryl sulfate and the drug load are the main factors that impact the granule, final blend and tablet core properties. Mannitol has less impact on the responses. High level of copovidone results in coarse granules and low fines. High level of sodium lauryl sulfate and low drug load contribute to faster dissolution rate. For all batches, the final blend flowability is acceptable and the final blends presented good tabletability regarding tensile strength and low ejection force.

Based on the above experiment, the following granule composition (Table 31)) is selected
Crospovidone ratio: at a middle ratio of 0.5 show a good compromise on granule particle size with less fines, low ejection force and fast tablet core DT and DR
Sodium lauryl sulfate: at a higher ratio level 0.04 is required for high dissolution rate
Mannitol SD 200 ratio (from the spray suspension): the presence of mannitol has low impact on granules, final blend and tablet physical properties. It was decided to remove the mannitol from granule composition for development
Drug load: at a lower ratio level (below 35%) contributes for fast dissolution rate

TABLE 31
granule composition

| | B: Sodium Lauryl | | |
|---|---|---|---|
| A: Copovidone ratio | Sulfate ratio | C: Mannitol | D: Drug load |
| 0.5 | 0.04 | 0 | <35% |

The drug ratio [compound (A):copovidone:SLS] corresponding to [2:1:0.08]

### Example 8: Film Coated Tablet

Using all the optimized parameters from the experiments in the former examples, the following film coated formulations were prepared as the optimal variant and good compromise between all variables.

Compound (a) Spray Suspension

| | | Amount per batch |
|---|---|---|
| | Amount per | size 105.263 Kg |
| | 1 Kg of | of spray |
| | spray | suspension |
| Component name | suspension | [Kg/batch] |
| Compound (A) | 190.00 | 20.0 |
| Kollidon V64 copovidone | 30.40 | 3.2 |
| Sodium Lauryl sulfate (Duponol C) | 0.76 | 0.08 |
| WET MEDIA MILLED SUSPENSION | 538.84 | 56.72 |
| | 760.00 | 80.00 |
| Kollidon V64 copovidone | 64.60 | 6.80 |
| Sodium Lauryl sulfate (Duponol C) | 6.84 | 0.72 |
| Purified water | 168.56 | 17.74 |
| SUSPENSION FOR SPRAY | 1000.00 | 105.263 |
| GRANULATION | | |

### Manufacturing Formula

| Amount per 1 tablet (mg/unit) | | | |
|---|---|---|---|
| 10 mg | 25 mg | 50 mg | 100 mg |

Preparation of milling suspension

| | | | | |
|---|---|---|---|---|
| Compound (A) | 10.000 | 25.000 | 50.000 | 100.000 |
| Copovidone | 1.600 | 4.000 | 8.000 | 16.000 |
| Sodium Lauryl Sulfate | 0.040 | 0.100 | 0.200 | 0.400 |
| Water purified | 28.360 | 70.900 | 141.800 | 283.600 |
| Milling suspension amount | 40.000 | 100.000 | 200.000 | 400.000 |

Preparation of final suspension

| | | | | |
|---|---|---|---|---|
| Copovidone | 3.400 | 8.500 | 17.000 | 34.000 |
| Sodium Lauryl Sulfate | 0.360 | 0.900 | 1.800 | 3.600 |
| Water purified | 8.773 | 21.934 | 43.867 | 87.734 |
| Final suspension for granulation | 52.533 | 131.334 | 262.667 | 525.334 |

Spray granulation

| | | | | |
|---|---|---|---|---|
| Mannitol SD | 52.100 | 39.500 | 79.000 | 158.000 |
| Granules amount | 67.500 | 78.000 | 156.000 | 312.000 |
| Cellulose MK GR (PH102) | 18.125 | 21.450 | 42.900 | 85.800 |
| Mannitol DC | 18.125 | 21.450 | 42.900 | 85.800 |
| Croscarmellose sodium | 7.500 | 7.800 | 15.600 | 31.200 |
| Sodium stearyl fumarate | 1.250 | 1.300 | 2.600 | 5.200 |
| Tablet core | 125.00 | 130.000 | 260.000 | 520.000 |

Preparation of coating suspension

| | | | | |
|---|---|---|---|---|
| Opadry white | 0.900 | 1.089 | 2.178 | 4.356 |
| Opadry yellow | 2.950 | 3.570 | 7.139 | 14.278 |
| Opadry red | 0.900 | 1.089 | 2.178 | 4.353 |
| Opadry black | 0.250 | 0.303 | 0.605 | 1.210 |
| Water purified | 28.333 | 34.248 | 68.567 | 137.134 |
| Amount of coating suspension | 33.333 | 40.335 | 80.667 | 161.334 |
| Film-coated tablet | 95.000 | 136.050 | 272.100 | 544.200 |

### Composition of Final Product

| Amount per 1 tablet (mg/unit) | | | |
|---|---|---|---|
| 10 mg | 25 mg | 50 mg | 100 mg |

granule (internal phase)

| | | | | |
|---|---|---|---|---|
| Compound (A) | 10.000 | 25.000 | 50.000 | 100.000 |
| Copovidone | 5.000 | 12.500 | 25.000 | 50.000 |
| Sodium Lauryl Sulfate | 0.400 | 1.000 | 2.000 | 4.000 |
| Mannitol SD | 52.100 | 39.500 | 79.000 | 158.000 |
| | 67.500 | 78.000 | 156.000 | 312.000 |

external phase

| | | | | |
|---|---|---|---|---|
| Cellulose MK GR (PH102) | 8.100 | 21.450 | 42.900 | 85.800 |
| Mannitol DC | 8.100 | 21.450 | 42.900 | 85.800 |
| Croscarmellose sodium | 5.400 | 7.800 | 15.600 | 31.200 |
| Sodium stearyl fumarate | 0.900 | 1.300 | 2.600 | 5.200 |
| Tablet core | 90.00 | 130.000 | 260.000 | 520.000 |

coating

| | | | | |
|---|---|---|---|---|
| Opadry white | 0.900 | 1.089 | 2.178 | 4.356 |
| Opadry yellow | 2.950 | 3.570 | 7.139 | 14.278 |
| Opadry red | 0.900 | 1.089 | 2.178 | 4.353 |
| Opadry black | 0.250 | 0.303 | 0.605 | 1.210 |
| Water purified | 28.333 | 34.248 | 68.567 | 137.134 |
| Amount of coating suspension | 33.333 | 40.335 | 80.667 | 161.334 |
| Film-coated tablet | 95.000 | 136.000 | 272.100 | 544.200 |

ratio of Compound (A)/Copovidone/SLS is 2:1:0.08

### Example 9: Manufacturing

The capsule and tablets final blends were prepared following a similar procedure as described in the flowchart hereinabove.
a. Dissolve the binder, e.g. polyvinylpyrrolidone-vinyl acetate copolymer, into water under stirring.
b. Add surfactant, e.g. sodium lauryl sulfate (SLS), to the solution of step a and dissolve under stirring.
c. Add Compound (A) to the solution of step b and suspend under stirring.
d. Perform milling, e.g. wet media milling, with the suspension of step c.
e. Dissolve required amounts of SLS and polyvinylpyrrolidone-vinyl acetate copolymer in the additional purified water under stirring.
f. Weigh required amount step d suspension and add to the solution of step e to complete the suspension for spraying, e.g. spray granulation.
g. Load the inert substrate (carrier particle), e.g. mannitol SD.
h. Perform spraying, e.g. spray granulation, by spraying the suspension from step e to the inert substrate, e.g. mannitol SD200, from step g.
i. The granule particles from step h were further mixed with some pharmaceutically acceptable excipients, for example, mannitol DS, sodium starch glycolate, polyvinylpyrrolidone-vinyl acetate copolymer, croscarmellose sodium.
j. The blend mixture from step i was introduced in a capsule or compressed to form a tablet.

### Example 10: Stability Experiment

### Stability Data of Capsules of Example 4

Stability data for the hard gelatin capsules of example 4 (10 mg, 25 mg and 50 mg) up to 24 months

### Stability Program:

The stability program tested the hard gelatin capsules of example 4 (10 mg, 25 mg and 50 mg) packaged in square high density polyethylene bottles with aluminum induction seal and child resistant screw cap closure (175 ml, 30 capsules) container under the following storage conditions: 5° C./ambient RH; 25° C./60% RH; 30° C./75% RH; 40° C./75% RH and 50° C./75% RH (RH relative humidity)

### Photostability Studies:

The photo stability testing was performed on the hard gelatin capsule of example 4 (10 mg, 25 mg and 50 mg) with the unpacked product according to the ICH guidelines for the ‘Photo stability testing of new active substances and medicinal products’ [ICH Q1B], using as light source the ICH Q1B option 2. A sample protected from light, run in parallel to the exposed sample was tested for use as a control.

The sample load of photostability was at least 1.2 million lux hours overall illumination and at least 200 watts hours/square meter near ultraviolet energy.

### Open Bottle:

This test was performed the hard gelatin capsules of example 4 stored in open glass dish. The samples were stored at 25° C./60% RH for up to 1 month. Afterwards the chemical and physical characteristics of the samples were analyzed.

### Freeze Thaw Cycle:

This test was performed with the hard gelatin capsules of example 4 (10 mg, 25 mg and 50 mg) packaged in square high density polyethylene bottles (HDPE) with aluminum induction seal and child resistant screw cap closure (175 ml, 30 capsules) container. The stability samples were stored for four complete freeze and thaw cycles (−20° C./ambient RH for 6 days, followed by 1 day at 25° C./60% RH). Samples were taken after 28 days and the chemical and physical characteristics were analyzed.

### Test Methods:

The following tests are performed as described in the table below:

| Appearance of content | Visual examination on a white base in diffused light | |
|---|---|---|
| by visual examination | | |
| Identity, Assay and | Determination by reverse phase HPLC method with gradient elution; | |
| degradation products | UV detection at 256 nm. | |
| by HPLC | Column | Acquity UPLC CSH C-18 or an equivalent |
| | | column |
| | | Length 100 mm, internal diameter 2.1 mm |
| | | and particle size of 1.7 μm |
| | Mobile phase A | Water/Acetonitrile/Trifluoroacetic acid |
| | | (950/50/0.5 v/v/v) |
| | Mobile phase B | Acetonitrile/Methanol/Water/Trifluoroacetic |
| | | acid (500/450/50/0.5 v/v/v/v) |
| | A gradient using the mobile phases A and B is applied. | |
| Uniformity of dosage | Ph. Eur. 2.9.40, JP and USP <905> (harmonized procedure); | |
| units by content | reversed phase HPLC with isocratic elution, with UV detection at | |
| uniformity | 256 nm. | |
| | Column | Symmetry C18 or an equivalent column |
| | | Length 50 mm, internal diameter 4.6 mm, |
| | | Particle size 3.5 μm |
| | Mobile phase | Water/Acetonitrile/TFA (650/350/1, v/v/v) |
| Dissolution by UV | Dissolution testing with apparatus 1 (basket) according to | |
| | Ph. Eur. 2.9.3 and USP <711>. | |
| | Determination of absorbance by UV detection at 256 nm. | |
| | Speed of rotation | 100 ± 4 rpm |
| | Test medium | 0.1M Hydrochloric acid |
| | Volume of test | 900 mL |
| | medium | |
| | Temperature | 37 ± 0.5° C. |

### Results of the Stability of Hard Gelatin Capsules

The hard gelatin capsules (10 mg, 25 mg and 50 mg) in HDPE bottles showed good physical and chemical stability when stored at 5° C./ambient RH, at 25° C./60% RH or at 30° C./75% RH, for up to 24 months. No significant changes in chemical and physical properties were observed.

The hard gelatin capsules (10 mg, 25 mg and 50 mg) in HDPE bottles showed good physical and chemical stability when stored at 40° C./75% RH up to 6 months. No significant changes in chemical and physical properties were observed.

The hard gelatin capsules (10 mg, 25 mg and 50 mg) in HDPE bottles showed good physical and chemical stability when stored at 50° C./75% RH for up to 1 month. No significant changes in chemical and physical properties were observed.

The photostability samples of the hard gelatin capsules (10 mg, 25 mg and 50 mg) in HDPE bottles showed good physical and chemical stability.

The freeze and thaw cycle samples of the hard gelatin capsules (10 mg, 25 mg and 50 mg) in HDPE bottles showed good physical and chemical stability.

The samples of open dish study of the hard gelatin capsules (10 mg, 25 mg and 50 mg) in HDPE bottles showed good physical and chemical stability.

### Stability Data of Film Coated Tablet (50 mg) of Example 8

### Stability Program:

The stability program tested the film coated tablet (10, 25, 50 and 100 mg) of example 8 packaged in square high density polyethylene bottles with aluminum induction seal and child resistant screw cap closure (175 ml, 30 capsules) container under the following storage conditions for up to 18 months:
5° C./ambient RH; 25° C./60% RH; 25° C./60% RH open; 30° C./75% RH; 30° C./75% RH open; 40° C./75% RH and 50° C./75% RH (RH relative humidity)

Photostability tests as well as freeze and thaw cycle tests were performed according the tests described above for the capsule.

Test Methods are performed as described above for the capsule.

### Results of Stability Tests:

The film coated tablet of example 8 (10, 25, 50 and 100 mg) showed good chemical and physical stability for up to 18 months when stored at 5° C./ambient RH, 25° C./60% RH, and 30° C./75% RH. No significant changes in chemical (assay and degradation products) and physical (appearance, thickness, diameter, dissolution rate, water content) properties were observed.

The film coated tablet of example 8 (10, 25, 50 and 100 mg) showed good chemical and physical stability for up to 6 months when stored at 40° C./75% RH in HDPE bottles. A slight increase in particle size was observed for the 10 mg and 25 mg tablet after storage at 40° C./75% RH in HDPE bottles (177.6 nm) when compared to the initial value (150.1 nm). No impact due to this slight increase is expected.

The film coated tablet of example 8 (10, 25, 50 and 100 mg) showed good chemical and physical stability for up to 1.5 months when stored at 50° C./75% in HDME bottles. No significant changes in chemical (assay and degradation products) and physical (appearance, thickness, diameter, dissolution rate, water content) properties except particle size for the 10 mg clinical batch were observed. There is a slight increase in particle size observed for the 10 mg tablet (from 150.5 nm at the initial time point to 196.0 nm) after storage for 1.5 months at 50° C./75% RH in HDPE bottles. However no impact due to this slight increase is expected.

The film coated tablet of example 8 (10, 25, 50 and 100 mg) showed good chemical and physical stability for up to 3 months when stored at 25° C./60% and 30° C./75% in open HDME bottles. No significant changes in chemical (assay and degradation products) and physical (appearance, thickness, diameter, dissolution rate, water content) properties. For the 100 mg tablet, a small increase in dissolution rate was observed (105%) after 3 months of storage at 30° C./75% in open HDME bottle. A slight increase of the particle size was observed for tablets stored for 3 months at 30° C./75% RH in open HDPE bottles compared to the initial value. For the 10 mg tablet, the particle size increased from 150.5 nm to 201.1 nm, while for the 25 mg tablet, it increased from 150.1 nm to 181.4 nm. Similarly for the 50 mg tablet, the particle size showed an increase from 148.9 nm to 178.7 nm while for the 100 mg tablet, it increased from 140.7 nm to 177.0 nm. No impact due to this slight increase is expected.

The photostability samples of the film-coated tablet (10, 25, 50 and 100 mg) in HDPE bottles showed good physical and chemical stability. No significant changes in chemical (assay and degradation products) and physical (appearance, thickness, diameter, dissolution rate, water content, particle size) properties. There is no effect of light on the stability of film-coated tablets.

The freeze and thaw cycle samples of the film coated tablet (10, 25, 50 and 100 mg) in HDPE bottles showed good physical and chemical stability.

### Stability of Crystalline Form was Evaluated by XRPD:

There was no change in the XRPD pattern observed for the film coated tablets of example 8 (10 mg, 25 mg, 50 mg and 100 mg) when stored for 9 months at 5° C./ambient RH, 25° C./60% RH and at 30° C./75% RH. Crystalline form (A) which is described in WO2020/234779 remains stable under those conditions. No conversion to other crystalline forms was observed

1. A pharmaceutical composition for oral administration comprising a granule particle said granule particle comprising:
(a) an inert substrate, and
(b) a mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, and at least one binder.

2. The pharmaceutical composition according to claim 1, wherein N-(3-(6-amino-5-(2-(N-methylacrylamido)ethoxy)pyrimidin-4-yl)-5-fluoro-2-methylphenyl)-4-cyclopropyl-2-fluorobenzamide is in a free form.

3. The pharmaceutical composition according to claim 1, wherein the (b) mixture optionally further comprises a surfactant.

4. The pharmaceutical composition according to claim 3, wherein the (b) mixture comprises a surfactant, further where the surfactant is layered onto the (a) inert substrate.

5. The pharmaceutical composition according to claim 4, wherein the (b) mixture and the surfactant is layered onto the (a) inert substrate using a spray granulation method.

6. The pharmaceutical composition according to claim 1, wherein the (a) inert substrate comprises a material which is selected from the group consisting of lactose, microcrystalline cellulose, mannitol, sucrose, starch, granulated hydrophilic fumed silica, or mixtures thereof, preferably the material, which is selected from the group consisting of lactose, mannitol, or mixtures thereof and most preferably the material is mannitol.

7. The pharmaceutical composition according to claim 1, wherein the binder is independently selected from the group consisting of polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, or a mixture thereof, preferably the binder is polyvinylpyrrolidone-vinyl acetate copolymer.

8. The pharmaceutical composition according to claim 4, wherein the surfactant is selected from the group consisting of sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof, preferably the surfactant is sodium lauryl sulfate.

9. The pharmaceutical composition according to claim 1, wherein the (b) mixture comprises N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, polyvinylpyrrolidone-vinyl acetate copolymer as a binder, and optionally sodium lauryl sulfate as a surfactant.

10. The pharmaceutical composition according to claim 1, wherein the weight ratio between N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof, and the binder is about [3:1], about [2:1] or about [1:1], or about [1:2] or about [1:3], preferably about [1:1] and more preferably about [2:1].

11. The pharmaceutical composition according to claim 4, wherein the weight ratio of N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof, the binder and the surfactant is [3:1:1], or about [3:1:0.5], or about [3:1:0.1], or about [2:1:1], or about [2:1:0.5], or about [2:1:0.1], or about [2:1:0.08], or about [2:1:0.05], or about [2:1:0.04], or about [2:1:0.03], or about [2:1:0.02], or about [1:1:0.5], or about [1:1:0.1], or about [1:1:0.07], or about [1:1:0.05], or about [1:1:0.04], or about [1:1:0.02], preferably, the ratio is about [2:1:1], or about [2:1:0.5], or about [2:1:0.1], or about [2:1:0.08], or about [2:1:0.05], or about [2:1:0.04], or about [2:1:0.03], or about [2:1:0.02], or about [1:1:0.5], or about [1:1:0.1], or about [1:1:0.07], or about [1:1:0.05], or about [1:1:0.04], or about [1:1:0.02], or about [1:3:0.1], or about [1:3:0.2], or about [1:1.5:0.25]; more preferably, the ratio is about [2:1:1], or about [2:1:0.08], or about [2:1:0.5], or about [2:1:0.1], or about [2:1:0.05], or about [2:1:0.04], or about [2:1:0.03], or about [2:1:0.02].

12. The pharmaceutical composition according to claim 1, wherein the binder is present in the (b) mixture in an amount of 25% w/w to about 100% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, preferably about 50% w/w or about 100% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof.

13. The pharmaceutical composition according to claim 3, wherein the (b) mixture further comprises a surfactant (e.g. Sodium lauryl sulfate) in an amount of 1% w/w to about 10% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, preferably about 4% w/w or about 5% w/w based on weight of N-(3-(6-amino-5-(2-(N-methylacrylamido)ethoxy)pyrimidin-4-yl)-5-fluoro-2-methylphenyl)-4-cyclopropyl-2-fluorobenzamide or a pharmaceutically acceptable salt thereof, or a free form thereof.

14. The pharmaceutical composition according to claim 1, wherein the particle size of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is less than 1000 nm.

15. The pharmaceutical composition according to claim 14, wherein the particle size of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is less than 500 nm.

16. The pharmaceutical composition according to claim 15, wherein the particle size of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is less than 350 nm, preferably less than 250 nm.

17. The pharmaceutical composition according to claim 14, wherein the particle size of N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof as measured by PCS is between about 100 nm to about 350 nm, preferably between about 110 nm and about 180 nm.

18. The pharmaceutical composition according to claim 1 further comprising an external phase wherein the external phase comprises one or more pharmaceutically acceptable excipient.

19. The pharmaceutical composition according to claim 18, wherein the one or more pharmaceutically acceptable excipient is selected from a filler, a disintegrating agent, a lubricating agent, and a gliding agent.

20. The pharmaceutical composition according to claim 18 wherein the external phase comprises one or more filler selected from calcium carbonate, sodium carbonate, lactose (e.g. lactose SD), mannitol (e.g. mannitol DC), magnesium carbonate, kaolin, cellulose (e.g. microcrystalline cellulose, powdered cellulose), calcium phosphate, or sodium phosphate, or mixture thereof preferably mannitol or cellulose or mixture thereof.

21. The pharmaceutical composition according to claim 18 wherein the external phase comprises one or more disintegrating agent selected from croscarmellose sodium, crospovidone, sodium starch glycolate, corn starch, or alginic acid, or mixture thereof.

22. The pharmaceutical composition according to claim 18 wherein the external phase comprises one or more lubricating agent selected from magnesium stearate, sodium stearyl fumarate, stearic acid or talc or mixture thereof.

23. The pharmaceutical composition according to claim 18 wherein the external phase comprises Mannitol and cellulose as fillers, sodium stearyl fumarate or magnesium stearate as lubricant, and croscarmellose sodium or sodium carbonate as disintegrating agent.

24. The pharmaceutical composition according to claim 18 wherein the external phase is present in a 20-50% w/w/amount of to the total weight of the composition, preferably 40% w/w/amount of to the total weight of the composition.

25. The pharmaceutical composition according to claim 1, wherein the pharmaceutical composition is further formulated into a final dosage form, optionally in the presence of at least one pharmaceutically acceptable excipient, and wherein said final dosage form is a capsule, a tablet, a sachet, or a stickpack.

26. The pharmaceutical composition according to claim 25, wherein the final dosage form is a capsule or preferably a tablet.

27. The pharmaceutical composition according to claim 25, wherein the capsule is selected from hard shell capsule, hard gelatin capsule, soft shell capsule, soft gelatin capsule, plant-based shell capsule, or a mixture thereof and wherein a tablet is preferably a film coated tablet.

28. A final dosage form which is a capsule formulation comprising a pharmaceutical composition of claim 1.

29. A final dosage form which is a tablet formulation comprising a pharmaceutical composition of claim 1.

30. A final dosage form according to claim 29, wherein N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof is present in an amount of about 10% w/w to about 25% w/w, preferably about 19% or about 20% based on the total weight of the final dosage form.

31. A final dosage form according to claim 29, wherein a filler is present in an amount of about 20 to about 40% w/w based on the total weight of the final dosage form.

32. A final dosage form according to claim 29, wherein a disintegrating agent is present in an amount of about 5% w/w to about 10% w/w, preferably about 5 or about 6% based on the total weight of the final dosage form.

33. A final dosage form according to claim 29 wherein the inert substrate is present in an amount of about 20% w/w to about 40% w/w, preferably about 30% based on the total weight of final dosage form.

34. A final dosage form according to claim 29 wherein the binder is present in an amount of about 5% w/w to about 25% w/w, preferably about 8 to about 12% w/w, based on the total weight of the final dosage form.

35. A final dosage form according to claim 29 wherein a lubricant is present in an amount of about 0.1 to about 2% w/w, preferably about 0.5% w/w to about 1.5% w/w based on the total weight of the final dosage form.

36. A final dosage form according to claim 29 wherein a surfactant is present in an amount of about 0.1% w/w to about 2.5% w/w, preferably about 0.2% w/w to about 0.8% w/w based on the total weight of the final dosage form.

37. A final dosage form according to claim 29 comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, in an amount of between about 0.5 mg to about 600 mg, e.g. about 5 mg to about 400 mg, e.g. about 10 mg to about 150 mg.

38. A final dosage form according to claim 29 comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, in an amount of about 0.5 mg, about 5 mg, about 10 mg, about 15 mg, about 20 mg, about 25 mg, about 50 mg, about 100 mg, about 150 mg, about 200 mg, about 250 mg, about 300 mg, about 350 mg, about 400 mg, about 450 mg, about 500 mg, or of about 600 mg, preferably in an amount of about 10 mg, about 25 mg, about 35 mg, about 50 mg, about 75 mg and about 100 mg.

39. A process for preparing the pharmaceutical composition according to claim 1, said process comprising the steps of:
i) Mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium, and
ii) Adding the said mixture (i) to the (a) inert substrate of the granule particles.

40. The process according to claim 39, wherein step (i) is performed in a wet milling chamber.

41. The process according to claim 39, wherein the liquid medium is an aqueous solution, e.g. purified water, preferably with a pH value between 5 and 8, and more preferably between 5 and 6.

42. The process according to claim 39, wherein the mixture of step (i) is dispersed onto the (a) inert substrate.

43. The process according to claim 39, wherein the process further comprises preparing the final dosage form by blending the mixture resulting from step (ii) with at least one pharmaceutically acceptable excipient.

44. The process according to claim 43, wherein the final dosage form is encapsulated or tableted.

45. The process according to claim 44, wherein the final dosage form is tableted and the resulting tablet is further film coated.

46. A process for preparing a suspension comprising mixing the (b) mixture comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, with a liquid medium.

47. A suspension comprising N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, at least one binder, and optionally a surfactant, in a liquid medium.

48. The suspension according to claim 47 wherein the particle size of said suspension is less than 1000 nm, preferably less than 500 nm, more preferably less than 350 nm and most preferably less than 250 nm.

49. The suspension according to claim 47 wherein the liquid medium is an aqueous solution, e.g. purified water, preferably with a pH value between 5 and 8, and more preferably between 5 and 6.

50. The suspension according to claim 47, wherein N-(3-(6-amino-5-(2-(N-methylacrylamido)ethoxy)pyrimidin-4-yl)-5-fluoro-2-methylphenyl)-4-cyclopropyl-2-fluorobenzamide, or a pharmaceutically acceptable salt thereof, or a free form thereof, is present in an amount of about 10% to about 40% of the total weight of the suspension, preferably about 20% or about 25% of the total weight of the suspension.

51. The suspension according to claim 47, wherein the at least one binder is present in an amount of about 3% to about 15% of the total weight of the suspension.

52. The suspension according to claim 47, wherein the surfactant is present in an amount of about 0.05% to about 1% of the total weight of the suspension.

53-55. (canceled)

56. A method of treating or preventing a disease or disorder mediated by BTK or ameliorated by the inhibition of BTK, comprising administering to a subject in need of such treatment or prevention, a pharmaceutical composition according to claim 1.

57. The method according to claim 56 wherein the disease or disorder mediated by BTK or ameliorated by the inhibition of BTK is selected from autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis, myasthenia gravis, Sjögren's syndrome, autoimmune hemolytic anemia, anti-neutrophil cytoplasmic antibodies (ANCA)-associated vasculitides, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including but not limited to multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease. Preferably, the disease or disorder mediated by BTK or ameliorated by the inhibition of BTK is selected from rheumatoid arthritis; chronic urticaria, preferably chronic spontaneous urticaria; Sjögren's syndrome, multiple sclerosis or asthma.
